(19) United States
(12) Patent Application Publication
(10) Pub. No.: US 2022/0033828 A1
(43) Pub. Date: Feb. 3, 2022

(54) GENETICALLY ENGINEERED CYANOBACTERIA FOR GROWTH IN UNSTERILIZED CONDITIONS USING ANTIBIOTIC-FREE SELECTION

(71) Applicant: Nanyang Technological University, Singapore (SG)

(72) Inventors: Tiago de Sousa Jorge Toscano SELÃO, Singapore (SG); Inger Birgitta NORLING, Singapore (SG); Peter Julian NIXON, Singapore (SG)

(21) Appl. No.: 17/311,048

(22) PCT Filed: Dec. 2, 2019
(86) PCT No.: PCT/SG2019/050593

§ 371 Date: Jun. 4, 2021

(30) Foreign Application Priority Data
Dec. 4, 2018 (SG) 10201810863R

(51) Int. Cl. C12N 15/52

(52) U.S. Cl. CPC C12N 15/52

(57) ABSTRACT

The present invention relates to methods of metabolic engineering cells to increase their ability to compete with contaminating microorganisms without the need for antibiotics. More particularly, the invention provides methods to engineer cyanobacteria to utilize melamine as nitrogen source, phosphite as phosphorous source, optionally also utilizing NADP+ over NAD+, and also provides genetically engineered cells made using such methods.

### FIELD OF THE INVENTION

The present invention relates to methods of metabolic engineering cells to increase their ability to compete with contaminating microorganisms without the need for antibiotics. More particularly, the invention provides methods to engineer cyanobacteria to utilize melamine as nitrogen source, phosphite as phosphorous source, optionally also utilizing NADP+ over NAD+, and also provides genetically engineered cells made using such methods.

### BACKGROUND OF THE INVENTION

As part of the effort to advance a carbon-neutral economy, cyanobacteria are increasingly being used for metabolic engineering. They are photoautotrophic organisms, able to grow with rather simple requirements—minimal media with inorganic nitrogen and phosphorus sources, using light for energy generation and CO₂ as the sole carbon input. In recent years, cyanobacteria have been shown to be able to produce a plethora of different molecules, from commodity chemicals such as lactate or ethanol [Angermayr et al., Applied and environmental microbiology. 78: 7098-106.10.1128/AEM.01587-12 (2012); Dexter et al., J Appl Microbiol. 119: 11-24.10.1111/jam.12821 (2015); Gordon et al., Metab Eng. 38: 170-179.10.1016/j.ymben.2016.07.007 (2016), to biofuels (e.g., free fatty acids [Kato et al., Biotechnol Biofuels. 10: 141.10.1186/s13068-017-0831-z (2017); Ruffing, Frontiers in bioengineering and biotechnology. 2: 17 (2014)] or butanol [Fathima et al., Biotechnol Biofuels. 11: 188.10.1186/s13068-018-1187-8 (2018); Shabestary et al., ACS Synth Biol. 7: 1669-1675 (2018)] to speciality chemicals such as farnesene [Halfmann et al., Appl Microbiol Biotechnol. 98: 9869 (2014)], squalene [Choi et al., ACS Synth Biol. 6: 1289-1295 (2017); Englund et al., PLoS One. 9: e90270.10.1371/journal.pone.0090270 (2014)] or limonene [Wang et al., Proceedings of the National Academy of Sciences of the United States of America. 113: 14225-14230 (2016)]. Fast-growing marine strains such as Synechococcus sp. PCC 7002 (henceforth “Syn7002”) are of special interest because it is able to grow in seawater (thus not competing for freshwater resources), withstand high light intensity and temperatures up to 40° C. (useful in large scale open-air facilities). Moreover, it is naturally transformable, has an optimal division time of roughly 4 hours and an available genome sequence [Begemann et al., PLoS One. 8: e76594.10.1371/journal.pone.0076594 (2013); Clark et al., Metab Eng. 47: 230-242 (2018); Frigaard et al., Methods Mol Biol. 274: 325-40 (2004); Gordon et al., Metab Eng. 38: 170-179.10.1016/j.ymben.2016.07.007 (2016); Ludwig and Bryant, Frontiers in microbiology. 2: 41 (2011); Ludwig and Bryant, Frontiers in microbiology. 3: 354 (2012); Markley et al., ACS Synth Biol. 4: 595 (2015); Perez et al., Journal of bacteriology. 10: 1128 (2016); Xu et al., Methods Mol Biol. 684: 273-93 (2011)].

Large scale cyanobacterial cultivation can be done in either semi-enclosed systems, such as hanging bags, or in open systems, be it raceway ponds or air-lifted stock ponds [Schoepp et al., Bioresour Technol. 166: 273-81 (2014)]. Closed systems have the advantage of higher controllability, less chance for culture contamination and generally higher growth yields. However, they are substantially more expensive to operate than open systems, with either air-lifted stock ponds or raceway ponds the most economically viable alternatives found so far [Schoepp et al., Bioresour Technol. 166: 273-81 (2014)]. Open systems have, on the other hand, the obvious disadvantage of being exposed to the environment and therefore are much more prone to contamination. The threat of contamination is usually minimized by using antibiotics and antibiotic resistant cyanobacteria strains. Operating a large open system with cyanobacterial strains carrying antibiotic-resistance genes introduces a severe biohazard should the cultures escape and, through horizontal gene transfer, may contribute to the spreading of antibiotic resistance to environmental pathogenic species.

Recently, the use of ecologically rare or xenobiotic sources of macronutrients has been explored as a means to generate selective pressure towards the growth of genetically modified organisms without the use of antibiotics [Kanda et al., J Biotechnol. 182: 68-73 (2014); Loera-Quezada et al., Plant Biotechnol J. 14: 2066 (2016); Pandeya et al., Plant Mol Biol. 95: 567-577 (2017); Polyviou et al., Environmental microbiology reports. 7: 824-30 (2015); Shaw et al., Science. 353: 583-6 (2016)] as well as to allow genetically modified plants to outcompete weeds, while consuming considerably less phosphorus [Lopez-Arredondo and Herrera-Estrella, Nat Biotechnol. 30: 889 (2012)]. Phosphite dehydrogenase (PtxD), an enzyme that converts phosphite, an ecologically rare form of phosphorus, into phosphate, has been introduced into a variety of organisms [Kanda et al., J Biotechnol. 182: 68-73 (2014); Lopez-Arredondo and Herrera-Estrella, Nat Biotechnol. 30: 889 (2012); Nahampun et al., Plant Cell Rep. 35: 1121-1132 (2016); Pandeya et al., Plant Mol Biol. 95: 567-577 (2017)]. A synthetic pathway to utilize melamine, a xenobiotic nitrogen-rich compound, has also been devised and introduced into different organisms [Shaw et al., Science. 353: 583-6 (2016)]. Introduction of the complete pathway (consisting of 6 enzymes) in Escherichia coli allowed the carrying strain to overcome deliberate contamination [Shaw et al., Science. 353: 583-6 (2016)].

In many cases these pathways or genes were introduced into target organisms with the help of antibiotic cassettes [Loera-Quezada et al., Plant Biotechnol J. 14: 2066 (2016); Motomura et al., ACS Synth Biol.10:1021 (2018); Shaw et al., Science. 353: 583-6 (2016)]. Even though this proves that the pathways confer an advantage to the organisms carrying them, the risk of horizontal gene transfer of the antibiotic resistance cassette(s) still exists.

In view of the above deficiencies, it is desirable to provide methods for producing engineered microorganisms that can more effectively compete with contaminants without a risk of antibiotic resistance gene transfer into the environment.

### SUMMARY OF THE INVENTION

The present invention provides methods of engineering cyanobacterial strains that are able to grow on melamine and/or phosphite as sole N and Pi sources, by using metabolic selection to drive their genomic integration without the need for antibiotic selection. Through laboratory evolution, seven different Synechococcus sp. PCC 7002 mutant strains were obtained that can grow on melamine as a sole N source. In addition, the use of a ptxD gene, or mutant thereof, and phosphite was shown also to be an efficient metabolic selectable marker in this cyanobacterial species. Cells transformed with melamine and phosphite metabolic pathways were able to grow using both melamine and phosphite as N and Pi sources, respectively, and could withstand and easily outcompete contamination, even in large excess.

The melamine mutant strains all had mutations affecting the triA gene and were designated Mel 1, having a Trp471stop mutation; Mel4, having a Leu88Phe mutation; Mel5, having a AGGAGA to AGAAGA mutation in the ribosome binding site (RBS); Mel6, having a Glu317Lys mutation; Mel7, having a His254Tyr mutation; Mel8, having a Ala355Val mutation; and Mel5evo, having a Thr218Asn mutation and a Val278Met mutation in triA, in addition to the same AGGAGA to AGAAGA mutation in the RBS as Mel5.

According to a first aspect, the present invention provides an isolated genetically engineered cyanobacterium, wherein the cyanobacterium has been transformed by at least one polynucleotide molecule; the at least one polynucleotide molecule comprising heterologous melamine utilization pathway genes, atzD, trzE, DUR1,2, trzC, guaD and triA operably linked to at least one promoter, wherein;
i) the triA gene comprises one or more mutations which encode amino acid substitutions, wherein the amino acid substitutions are at positions selected from the group comprising Leu88Phe, His254Tyr, Glu317Lys, Ala355Val, Trp471Stop and the combination of Thr218Asn and Val278Met;
and/or
ii) the triA gene has a ribosome binding site (RBS) comprising a AGGAGA to AGAAGA mutation,
   wherein said genetically engineered cyanobacterium has no heterologous antibiotic resistance genes.

According to another aspect, the present invention provides an isolated genetically engineered cyanobacterium, wherein the cyanobacterium has been transformed by at least one polynucleotide molecule; the at least one polynucleotide molecule comprising a heterologous phosphite dehydrogenase (ptxD) gene operably linked to a promoter, wherein the ptxD gene comprises a polynucleotide sequence set forth in SEQ ID NO: 89 (native), SEQ ID NO: 90 (MelPhi), or SEQ ID NO: 91 (NADP), wherein said genetically engineered cyanobacterium has no heterologous antibiotic resistance genes.

According to another aspect, the present invention provides a recombinant vector comprising melamine pathway genes triA, DUR1,2, atzD, trzC, trzE, and guaD, operably linked to at least one promoter, wherein
i) the triA gene comprises one or more mutations which encode amino acid substitutions, wherein the amino acid substitutions are at positions selected from the group comprising Leu88Phe, His254Tyr, Glu317Lys, Ala355Val, Trp471Stop, and the combination of 254His and Val278Met;
and/or
ii) the triA gene has a ribosome binding site (RBS) comprising a AGGAGA to AGAAGA mutation,
   wherein the vector lacks antibiotic resistance genes.

In some embodiments the recombinant vector further comprises a polynucleotide comprising a heterologous phosphite dehydrogenase (ptxD) gene operably linked to a promoter.

According to another aspect, the present invention provides a method of expressing a product in a genetically engineered cyanobacterium cell, comprising the steps:
a) culturing a plurality of genetically engineered cyanobacterium cells comprising heterologous melamine utilization pathway genes and at least one exogenous polynucleotide comprising an expressible polynucleotide encoding an RNA and/or a protein product, according to any aspect of the invention, in medium where there is no antibiotic and melamine is the nitrogen source, wherein culturing favours growth of cyanobacterium cells that metabolise melamine,
b) culturing said genetically engineered cyanobacterium cells under conditions for expression of said product.
   According to another aspect, the present invention provides a method of expressing a product in a genetically engineered cyanobacterium cell, comprising the steps:
a) culturing a plurality of genetically engineered cyanobacterium cells, comprising heterologous melamine utilization pathway genes and phosphite metabolism genes and at least one exogenous polynucleotide comprising an expressible polynucleotide encoding an RNA and/or a protein product, in medium where there is no antibiotic, melamine is the nitrogen source and phosphite is the phosphorous source, wherein culturing favours growth of cyanobacterium cells that metabolise melamine and phosphite;
b) culturing said genetically engineered cyanobacterium cells under conditions for expression of said product.

### BRIEF DESCRIPTION OF THE FIGURES

FIG. 1 shows an overview of melamine selection tool. (A) Melamine utilization pathway reactions. One mol of melamine yields 6 mol ammonia and 3 mol carbon dioxide. (B) Schematic view of the melamine utilization operon. Primers indicated were used to confirm full genome integration of the pathway. Different parts are not to scale (C) 0.6% agarose gel of PCR reaction using primers stated in A. (see Table 1 for sequences)

FIG. 2 shows growth of melamine utilizing strains in melamine containing medium. (A) Growth curve of wildtype (WT) Syn7002 and melamine utilizing strains. (B) Samples of cultures 48 hours after inoculation. Factors for converting OD₇₃₀ to grams dry cell weight (gDCW)·L⁻¹ were calculated for all strains and can be found in Table 2.

FIGS. 3A-3B shows a schematic representation of mutations to the triA locus in different melamine utilizing strains, as found by Illumina sequencing. Mel1 has a mutation 4 amino acids before the original stop codon (FIG. 3A). Multiple sequence alignment of the triA locus in the different melamine utilizing strains (FIG. 3B).

FIG. 4 shows LC-MS/MS quantification of melamine pathway intermediates in spent culture medium at the time points indicated. (A) Melamine; (B) Ammeline; (C) Ammelide; (D) Cyanuric acid. Quantification for WT culture inoculated in AD7-Mel medium is also included, as a control. Notice the difference in scale for melamine (in mM) and remaining intermediates (in μM). Error bars may not be apparent due to scale.

FIG. 5 shows growth curves for the Mel5 strain, grown in AD7-Mel medium containing either 2 mM or 4 mM melamine.

FIG. 6 shows growth of phosphite utilizing strains in phosphate (Pho) and phosphite (Phi) containing AD7 medium. (A) Growth curve of WT Syn7002 (left) and phosphite (right) utilizing strain in AD7 medium containing Pho or different concentrations of Phi, as indicated. (B) Detail of culture samples at 48 hours after inoculation. Factors for converting OD₇₃₀ to grams dry cell weight (gDCW)·L⁻¹ can be found in Table 2.

FIG. 7 shows an overview of the phosphite selection tool. (A) Top—Detail of pSJ135, including primers used for chromosomal integration PCR. Bottom—Detail of construct pSJ141, which uses phosphite to drive chromosomal integration of a heterologous gene (YFP). (B) 0.8% agarose gel of PCR showing genome integration of ptxD gene and ptxD-driven integration of the YFP gene, in both WT and Mel5 backgrounds. (C) YFP fluorescence in strains transformed with pSJ135 and pSJ141 vs. respective background strains.

FIGS. 8A-8C show knock-out of putative phosphonate transporter homologues A0336 (top) and G0143 (bottom) in Syn7002. (FIG. 8A) Schematic representation of knock-out construct plasmids pSJ156 (top) and pSJ157 (bottom). Individual elements are not to scale. (FIG. 8B) Segregation gels for A0935-ptxD putative phosphonate transporter homologue knock-out strains. (FIG. 8C) Dilution plating of A0935-ptxD parental strain and derivative knock-out strains, in either AD7-Pho 1× (left) or AD7-Phi 20× (right). Note: ΔA-ΔA0336::SpR; AG-ΔG0143::GmR

FIG. 9 shows characterization of the melamine and phosphite utilizing strain. (A) Growth curves of WT and Mel5-A0935ptxD (“MelPhi”) strains in either regular AD7 medium or AD7-Mel Phi 20×. (B) Detail of culture samples at 48 hours post inoculation. Factors for OD₇₃₀ to grams dry cell weight (gDCW)·L⁻¹ conversion were calculated for the MelPhi strain in AD7-Mel Pho 1× or AD7-Mel Phi 20× and can be found in Table 2. (C) Growth curves of a strain expressing YFP in the Syn7002 WT background (“YFP pure”, grown in regular AD7); the MelPhi strain (“MelPhi pure”, lacking YFP, grown in AD7-Mel Phi 20×); or mixed cultures of the two strains combined in at a cell ratio of 6:1 YFP (in WT background) to MelPhi (lacking YFP), in AD7-Mel Phi 20×, measured by flow cytometry. “YFP mix” is the cell count of YFP strain in the mix, and “MelPhi mix” is the cell count of MelPhi strain in the mix.

FIG. 10 shows a growth curve of WT, Mel5 and Re-Mel5 strains in either normal AD7-NO₃ or AD7-Mel.

FIG. 11 shows growth curves of a strain expressing YFP in the Syn7002 WT background (“YFP pure”, grown in regular AD7), the MelPhi strain (“MelPhi pure”, lacking YFP, grown in AD7-Mel Phi 20×), or mixed cultures of the two strains (“YFP mix” and “MelPhi mix”), combined in a cell ratio of 10:1 YFP (in WT background) to MelPhi (lacking YFP), in AD7-Mel Phi 20×, measured by flow cytometry.

FIG. 12 shows gating strategy used for cell counts in the contamination experiment. Data shown is for one of the 10:1 mixed culture experiments, at T=100 hours. Left-Gates drawn on dot plots for (top to bottom) YFP and MelPhi pure cultures and the mixed culture; Middle-Histogram plots for the same samples; Right-Dot plots for the same samples using YFP vs. forward scatter, used for quantification. SSC—side scatter; FSC—forward scatter

FIG. 13 shows growth curve of Syn7002 WT and melamine and phosphite utilizing strains in AD7 with either nitrate (NO₃) or melamine (Mel) and phosphate (Pho) or phosphite (Phi). All plates were grown at 30° C., 80 μE·m⁻²·s⁻¹ and 1% CO₂ for 5 days.

FIG. 14 shows growth curve of the MelPhi strain in two baffled 1 L Erlenmeyer flasks over a period of 11 days in AD7-Mel Phi 20× (total volume of culture 2 L).

FIG. 15 shows growth curves for the MelPhi strain (MelPhi WT) and a derivative of the MelPhi strain (MelPhiAQ) in which the PtxD enzyme was mutated to use NADP+ instead of NAD+(as cyanobacteria have more NADP+ than NAD+). The strains were grown using a fed-batch strategy, adding melamine every day (600 μL of 20 mM melamine stock into a 12 mL culture) to continue growing to higher densities. The highest density reached was about OD₇₃₀ 70.

FIG. 16 shows growth curves of the Mel5 strain and a Mel5 strain evolved in 12 mM melamine (designated “Mel5evo”). The Mel5 strain cannot grow in 12 mM melamine but the Mel5evo strain can and grows to an OD₇₃₀ of about 50.

### DEFINITIONS

Certain terms employed in the specification, examples and appended claims are collected here for convenience.

The terms “amino acid” or “amino acid sequence,” as used herein, refer to an oligopeptide, peptide, polypeptide, or protein sequence, or a fragment of any of these, and to naturally occurring or synthetic molecules. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

The term “gene mutation” as used herein is defined as one which has at least one nucleotide sequence that varies from a wild-type sequence via substitution, deletion or addition of at least one nucleic acid that may enhance the activity of the gene or that may result in the encoding of an amino acid sequence of a protein that is relatively more active compared to the wild-type protein. For example, at least one native or wild-type melamine deaminase (triA) gene and/or its ribosome binding site (RBS) AGGAGA may be mutated to increase melamine metabolism.

The term “isolated” is herein defined as a biological component (such as a nucleic acid, peptide or protein) that has been substantially separated, produced apart from, or purified away from other biological components in the cell of the organism in which the component naturally occurs, i.e., other chromosomal and extra-chromosomal DNA and RNA, and proteins. Nucleic acids, peptides and proteins which have been isolated thus include nucleic acids and proteins purified by standard purification methods. The term also embraces nucleic acids, peptides and proteins prepared by recombinant expression in a host cell as well as chemically synthesized nucleic acids.

The phrases “nucleic acid” or “nucleic acid sequence,” as used herein, refer to an oligonucleotide, nucleotide, polynucleotide, or any fragment thereof, to DNA or RNA of genomic or synthetic origin which may be single-stranded or double-stranded and may represent the sense or the antisense strand, to peptide nucleic acid (PNA), or to any DNA-like or RNA-like material.

As used herein, the term “operably linked” means that the components to which the term is applied are in a relationship that allows them to carry out their inherent functions under suitable conditions. For example, a control sequence which is “operably linked” to a protein coding sequence is ligated thereto, so that expression of the protein coding sequence is achieved under conditions compatible with the transcriptional activity of the control sequences. By way of an example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

The term ‘mutant’ as used herein, means a polynucleotide may encode a mutant of an exemplified catalytic enzyme which retains activity, or may have a mutation, for example in its RBS that enhances catalytic enzyme production. A “mutant” of a catalytic enzyme, as used herein, refers to an amino acid sequence that is altered by one or more amino acids. The mutant may have “conservative” changes, wherein a substituted amino acid has similar structural or chemical properties (e.g., replacement of leucine with isoleucine). More rarely, a mutant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing catalytic activity may be found using computer programs well known in the art, for example, DNASTAR software. In some embodiments, mutant enzymes are at least 80%, 85%, 90%, 95%, 96%, 97%, 98%, 99%, or more, preferably at least 90%, homologous or identical at the amino acid level to an exemplary amino acid sequence described herein (e.g., melamine deaminase) or a functional fragment thereof—e.g., over a length of about: 50, 55, 60, 65, 70, 75, 80, 85, 90, 95, or 100%, preferably at least 90%, of the length of the mature reference sequence, yet retain catalytic activity. Preferably said variant enzymes have at least 90% identity at the amino acid level and retain catalytic activity. An exemplary melamine deaminase mutant is represented as SEQ ID NO: 64 in Mel7 (SEQ ID NO: 87) which has His254Tyr substitution that increases activity. It is possible that 254Tyr could be replaced by another amino acid (conservative substitution) and retain activity.

A vector can include one or more catalytic enzyme nucleic acid(s) in a form suitable for expression of the nucleic acid(s) in a host cell. Preferably the recombinant expression vector includes one or more regulatory sequences operatively linked to the nucleic acid sequence(s) to be expressed. The term “regulatory sequence” includes promoters, enhancers, ribosome binding sites and/or IRES elements, and other expression control elements (e.g., polyadenylation signals). Regulatory sequences include those which direct constitutive expression of a nucleotide sequence such as the P_{c223} promoter disclosed in the Examples herein. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., catalytic enzyme proteins).

The recombinant expression vectors of the invention can be designed for expression of catalytic enzyme proteins in prokaryotic or eukaryotic cells, more particularly prokaryotic cells. For example, polypeptides of the invention can be expressed in bacteria (e.g., cyanobacteria) or yeast cells. Suitable host cells are discussed further in Goeddel, (1990) Gene Expression Technology: Methods in Enzymology 185, Academic Press, San Diego, Calif.

The methods described hereinbefore make use of enzymes to catalyse a sequence of reactions. While these reactions may be performed individually or, more particularly, two or more of them in combination, it is particularly preferred that all of the reactions are combined into a cascade reaction sequence that provides the product from the initial starting material in one pot, thereby eliminating the need for isolation of the intermediates and, potentially, increasing the overall yield of the reaction sequence.

In this invention, novel engineered bacteria do not contain antibiotic resistance genes and instead utilize melamine and phosphite to compete against contaminants. Moreover, the engineered cells of the invention comprise mutations in the triA gene and/or in its RBS that improve growth of the organism, some of which can grow strongly in 12 mM melamine. In addition, the phosphite metabolism gene ptxD can be mutated to utilize NADP+ instead of NAD+.

### DETAILED DESCRIPTION OF THE INVENTION

Bibliographic references mentioned in the present specification are for convenience listed in the form of a list of references and added at the end of the examples. The whole content of such bibliographic references is herein incorporated by reference but their mention in the specification does not imply that they form part of the common general knowledge.

According to a first aspect, the present invention provides an isolated genetically engineered cyanobacterium, wherein the cyanobacterium has been transformed by at least one polynucleotide molecule; the at least one polynucleotide molecule comprising heterologous melamine utilization pathway genes, atzD, trzE, DUR1,2, trzC, guaD and triA operably linked to at least one promoter, wherein;
i) the triA gene comprises one or more mutations which encode amino acid substitutions, wherein the amino acid substitutions are at positions selected from the group comprising Leu88Phe, His254Tyr, Glu317Lys, Ala355Val, Trp471Stop and the combination of Thr218Asn and Val278Met;
and/or
ii) the triA gene has a ribosome binding site (RBS) comprising a AGGAGA to AGAAGA mutation,
   wherein said genetically engineered cyanobacterium has no heterologous antibiotic resistance genes.

In some embodiments the cyanobacterium is Synechococcus sp. Syn7002.

In some embodiments the triA gene encodes an amino acid sequence selected from the group comprising SEQ ID NO: 56 (native, Mel5), SEQ ID NO: 58 (Mel1), SEQ ID NO: 60 (Mel4), SEQ ID NO: 62 (Mel6), SEQ ID NO: 64 (Mel7), SEQ ID NO: 66 (Mel8) and SEQ ID NO: 68 (Mel5evo).

In some embodiments the triA gene polynucleotide sequence has at least 80%, at least 85%, at least 90%, at least 95% sequence identity or 100% sequence identity to the polynucleotide sequence of the triA gene selected from the group comprising SEQ ID NO: 57; SEQ ID NO: 59 (Mel1); SEQ ID NO: 61 (Mel4); SEQ ID NO: 63 (Mel6); SEQ ID NO: 65 (Mel7); SEQ ID NO: 67 (Mel8); SEQ ID NO: 69 (Mel5evo) and SEQ ID NO: 70 (Mel5).

It would be understood that due to the redundancy in the genetic code, a nucleic acid sequence may have less than 100% identity and still encode the same amino acid sequence.

In some embodiments the triA gene comprises a polynucleotide sequence selected from the group comprising SEQ ID NO: 57 (native), SEQ ID NO: 59 (Mel1), SEQ ID NO: 61 (Mel4), SEQ ID NO: 63 (Mel6), SEQ ID NO: 65 (Mel7), SEQ ID NO: 67 (Mel8), SEQ ID NO: 69 (Mel5evo) and SEQ ID NO: 70 (Mel5 codon opt).

Preferably, the triA gene comprises the polynucleotide sequence set forth in SEQ ID NO: 69 or SEQ ID NO: 70.

In some embodiments the heterologous trzE gene comprises a polynucleotide sequence at least 80%, at least 85%, at least 90%, at least 95% sequence identity or 100% sequence identity to the polynucleotide sequence set forth in SEQ ID NO: 71 or 72; the trzC gene comprises a polynucleotide sequence at least 80%, at least 85%, at least 90%, at least 95% sequence identity or 100% sequence identity to the polynucleotide sequence set forth in SEQ ID NO: 73 or 74; the DUR1,2 gene comprises a polynucleotide sequence at least 80%, at least 85%, at least 90%, at least 95% sequence identity or 100% sequence identity to the polynucleotide sequence set forth in SEQ ID NO: 75 or 76; the atzD gene comprises a polynucleotide sequence at least 80%, at least 85%, at least 90%, at least 95% sequence identity or 100% sequence identity to the polynucleotide sequence set forth in SEQ ID NO: 77 or 78; and/or the guaD gene comprises a polynucleotide sequence at least 80%, at least 85%, at least 90%, at least 95% sequence identity or 100% sequence identity to the polynucleotide sequence set forth in SEQ ID NO: 79, 80 or 81 (Arg352Ser).

In some embodiments the heterologous trzE gene comprises a polynucleotide sequence set forth in SEQ ID NO: 71 or 72 (codon optimized); the trzC gene comprises a polynucleotide sequence set forth in SEQ ID NO: 73 or 74 (codon optimized); the DUR1,2 gene comprises a polynucleotide sequence set forth in SEQ ID NO: 75 or 76 (codon optimized); the atzD gene comprises a polynucleotide sequence set forth in SEQ ID NO: 77 or 78 (codon optimized); and/or the guaD gene comprises a polynucleotide sequence set forth in SEQ ID NO: 79, 80 (codon optimized) or 81 (Arg352Ser).

In some embodiments the atzD gene is from Pseudomonas sp. strain ADP; the trzE gene is from Rhodococcus sp. Mel; the DUR1,2 gene is from S. cerevisiae; the trzC gene is from A. citrulli NRRL B-12227; the guaD gene is from E. coli K-12 and the triA gene is from A. citrulli NRRL B-12227.

In some embodiments each of said melamine utilization pathway genes has a ribosome binding site (RBS). An example of a suitable RBS has the polynucleotide sequence AGGAGA. Advantageously, a mutant RBS comprising the polynucleotide sequence AGAAGA may be used. More particularly, this mutant RBS is linked to the triA gene. It would be understood that an IRES may be suitable in place of one or more of the RBS linked to the atzD, trzE, DUR1,2, trzC and guaD genes.

In some embodiments said at least one promoter is a constitutive promoter. It would be understood that there are known promoters, such as P_{trc}, P_{psbA}, P_{cpcB} and P_{c223}, that would be suitable to drive expression of the melamine pathway genes. Preferably, the promoter is a strong promoter such as P_{c223} [Markley et al., ACS Synth Biol. 4: 595 (2015)].

In some embodiments said constitutive promoter is P_{c223} (SEQ ID NO: 82).

In some embodiments said heterologous melamine utilization pathway genes are expressed from a single promoter as a part of a gene operon.

In some embodiments the gene operon polynucleotide sequence is selected from the group comprising SEQ ID NO: 83 (Mel1 strain), SEQ ID NO: 84 (Mel4 strain), SEQ ID NO: 85 (Mel5 strain), SEQ ID NO: 86 (Mel6 strain), SEQ ID NO: 87 (Mel7 strain) and SEQ ID NO: 88 (Mel8 strain).

In some embodiments the at least one polynucleotide molecule further comprises a polynucleotide comprising a heterologous phosphite dehydrogenase (ptxD) gene operably linked to a promoter. Results showed that ptxD could be used on its own to select for recombinant strains without the need for antibiotic selection. The ptxD gene could also be used in combination with melamine pathway genes (MelPhi strains) in a more stringent selection method to produce strains that compete strongly with contaminating bacteria lacking these heterologous genes. Moreover, a mutant form of ptxD was generated that allowed the engineered strain to utilize NADP+ over NAD+.

In some embodiments the ptxD gene comprises a polynucleotide sequence set forth in SEQ ID NO: 89 (native), SEQ ID NO: 90 (MelPhi), or SEQ ID NO: 91 (NADP).

In some embodiments the promoter linked to the ptxD gene may be selected from a group comprising P_{trc}, P_{psbA}, P_{cpcB} and P_{c223}. In some embodiments the promoter linked to the ptxD gene is psbA comprising the polynucleotide sequence set forth in SEQ ID NO: 92.

In some embodiments said heterologous phosphite dehydrogenase (ptxD) gene is expressed from a single promoter as a part of a gene operon, wherein the operon polynucleotide sequence is set forth in SEQ ID NO: 93.

In some embodiments the isolated genetically engineered cyanobacterium of the invention further comprises an exogenous polynucleotide comprising an expressible polynucleotide encoding an RNA and/or a protein product.

According to another aspect, the present invention provides an isolated genetically engineered cyanobacterium, wherein the cyanobacterium has been transformed by at least one polynucleotide molecule; the at least one polynucleotide molecule comprising a heterologous phosphite dehydrogenase (ptxD) gene operably linked to a promoter, wherein the ptxD gene comprises a polynucleotide sequence set forth in SEQ ID NO: 89 (native), SEQ ID NO: 90 (MelPhi), or SEQ ID NO: 91 (NADP),

and wherein said genetically engineered cyanobacterium has no heterologous antibiotic resistance genes.

Preferably the ptxD gene comprises a polynucleotide sequence set forth in SEQ ID NO: 90, or SEQ ID NO: 91.

According to another aspect, the present invention provides a recombinant vector comprising melamine pathway genes triA, DUR1,2, atzD, trzC, trzE, and guaD, operably linked to at least one promoter, wherein
i) the triA gene comprises one or more mutations which encode amino acid substitutions, wherein the amino acid substitutions are at positions selected from the group comprising Leu88Phe, His254Tyr, Glu317Lys, Ala355Val, Trp471Stop, and the combination of Thr218Asn and Val278Met;
and/or
ii) the triA gene has a ribosome binding site (RBS) comprising a AGGAGA to AGAAGA mutation,
   wherein the vector lacks antibiotic resistance genes.

In some embodiments the triA gene encodes an amino acid sequence selected from the group comprising SEQ ID NO: 56 (native, Mel5), SEQ ID NO: 58 (Mel1), SEQ ID NO: 60 (Mel4), SEQ ID NO: 62 (Mel6), SEQ ID NO: 64 (Mel7), SEQ ID NO: 66 (Mel8) and SEQ ID NO: 68 (Mel5evo).

In some embodiments the triA gene comprises a polynucleotide sequence selected from the group comprising SEQ ID NO: 57 (native), SEQ ID NO: 59 (Mel1), SEQ ID NO: 61 (Mel4), SEQ ID NO: 63 (Mel6), SEQ ID NO: 65 (Mel7), SEQ ID NO: 67 (Mel8), SEQ ID NO: 69 (Mel5evo) and SEQ ID NO: 70 (Mel5).

In some embodiments the heterologous gene trzE comprises a polynucleotide sequence set forth in SEQ ID NO: 71 or 72 (codon optimized); trzC comprises a polynucleotide sequence set forth in SEQ ID NO: 73 or 74 (codon optimized); DUR1,2 comprises a polynucleotide sequence set forth in SEQ ID NO: 75 or 76 (codon optimized); atzD comprises a polynucleotide sequence set forth in SEQ ID NO: 77 or 78 (codon optimized); guaD comprises a polynucleotide sequence set forth in SEQ ID NO: 79, 80 (codon optimized) or 81 (Arg352Ser).

In some embodiments the atzD gene is from Pseudomonas sp. strain ADP; the trzE gene is from Rhodococcus sp. Mel; the DUR1,2 gene is from S. cerevisiae; the trzC gene is from A. citrulli B-12227; the guaD gene is from E. coli and the triA gene is from A. citrulli B-12227.

In some embodiments each of said melamine utilization pathway genes has a ribosome binding site (RBS). An example of a suitable RBS has the polynucleotide sequence AGGAGA. Advantageously, a mutant RBS comprising the polynucleotide sequence AGAAGA may be used. More particularly, this mutant RBS is linked to the triA gene. It would be understood that an IRES may be suitable in place of one or more of the RBS linked to the atzD, trzE, DUR1,2, trzC and guaD genes.

In some embodiments said at least one promoter is a constitutive promoter. It would be understood that there are known promoters, such as P_{trc}, P_{psbA}, P_{cpcB} and P_{c223}, that would be suitable to drive expression of the melamine pathway genes. Preferably, the promoter is a strong promoter such as P_{c223} [Markley et al., ACS Synth Biol. 4: 595 (2015)].

In some embodiments said constitutive promoter is P_{c223} (SEQ ID NO: 82).

In some embodiments said heterologous melamine utilization pathway genes are expressed from a single promoter as a part of a gene operon.

In some embodiments the gene operon polynucleotide sequence is selected from the group comprising SEQ ID NO: 83 (Mel1), SEQ ID NO: 84 (Mel4), SEQ ID NO: 85 (Mel5), SEQ ID NO: 86 (Mel6), SEQ ID NO: 87 (Mel7) and SEQ ID NO: 88 (Mel8).

In some embodiments the at least one polynucleotide molecule further comprises a polynucleotide comprising a heterologous phosphite dehydrogenase (ptxD) gene operably linked to a promoter.

In some embodiments the ptxD gene comprises a polynucleotide sequence selected from the group comprising SEQ ID NO: 89 (native), SEQ ID NO: 90 (MelPhi) and SEQ ID NO: 91 (NADP).

In some embodiments the promoter linked to the ptxD gene is selected from a group comprising P_{trc}, P_{psbA}, P_{cPCB} and P_{c223}. In some embodiments the promoter linked to the ptxD gene is psbA comprising the polynucleotide sequence set forth in SEQ ID NO: 92.

In some embodiments the recombinant vector further comprises an exogenous polynucleotide comprising an expressible polynucleotide encoding an RNA and/or a protein product.

According to another aspect, the present invention provides a method of expressing a product in a genetically engineered cyanobacterium cell, comprising the steps:
a) culturing a plurality of genetically engineered cyanobacterium cells, comprising heterologous melamine utilization pathway genes and at least one exogenous polynucleotide comprising an expressible polynucleotide encoding an RNA and/or a protein product according to any aspect of the invention, in medium where there is no antibiotic and melamine is the nitrogen source, wherein culturing favours growth of cells that metabolise melamine,
b) culturing said genetically engineered cyanobacterium cells under conditions for expression of said product.

According to another aspect, the present invention provides a method of expressing a product in a genetically engineered cyanobacterium cell, comprising the steps:
a) culturing a plurality of genetically engineered cyanobacterium cells, comprising heterologous melamine utilization pathway genes and phosphite metabolism genes and at least one exogenous polynucleotide comprising an expressible polynucleotide encoding an RNA and/or a protein product according to any aspect of the invention, in medium where there is no antibiotic, melamine is the nitrogen source and phosphite is the phosphorous source, wherein culturing favours growth of cyanobacterium cells that metabolise melamine and phosphite,
b) culturing said genetically engineered cyanobacterium cells under conditions for expression of said product.

In some embodiments said expressed product is capable of converting a substrate into another product. The said product may, for example, be an enzyme that can catalyse conversion of a substrate in the culture into another product. For example, said expressed product may be an enzyme such as farnesene synthase, which can convert CO₂ and H₂O into farnesene (C₁₅H₂₄).

In some embodiments the medium comprises melamine at a concentration of at least 1 mM, at least 2 mM, at least 4 mM, at least 6 mM, at least 8 mM, at least 10 mM, at least 12 mM, at least 14 Mm, or at least 16 mM. In some embodiments the concentration of melamine in the medium is selected from a concentration in the range of about 2 mM to about 12 mM.

In some embodiments the method further comprises isolating said product expressed in the genetically engineered cyanobacterium cell.

Having now generally described the invention, the same will be more readily understood through reference to the following examples that are provided by way of illustration, and are not intended to be limiting of the present invention.

### EXAMPLES

### Example 1: Methods

Standard molecular biology techniques known in the art and not specifically described were generally followed as described in Green and Sambrook and Russel, Molecular Cloning: A Laboratory Manual, Cold Springs Harbor Laboratory, New York (2012).

### Cell Growth Conditions

Synechococcus sp. PCC 7002 (a kind gift from Prof. Donald Bryant, Penn State University, USA) was grown photoautotrophically in medium A [Stevens et al., J Phycol. 9: 427-430 (1973)] using D7 micronutrients [Arnon et al., Biochim Biophys Acta. 357: 231-45 (1974)], supplemented with either 12 mM sodium nitrate (AD7-NO₃), 4 mM cyanurate (AD7-Cya) or 2 mM melamine (AD7-Mel) as indicated and vitamin B₁₂ (0.01 mg/L). For phosphite-utilizing strains, potassium dihydrogen phosphate (Pho) was substituted by potassium dihydrogen phosphite (Phi, Rudong Huayun Chemical Co., Ltd., Jiangsu, China), at 0.370 mM (Pho 1× or Phi 1×). Solid medium was prepared by supplementing the above media with 1.2% (w/v) Bacto-Agar (BD Diagnostics) and 1 g/L sodium thiosulfate.

For growth experiments, liquid pre-cultures of either Syn7002 WT (grown in AD7-NO₃) or melamine-growing strains (grown in AD7-Mel) were cultivated at a low-light intensity of 50 μmol photons·m⁻²·s⁻¹ at 38° C., 1% CO₂, 160 rpm, until an OD₇₃₀ between 4 and 6 (late logarithmic phase under low-light conditions). Cells were pelleted and washed twice with AD7-Mel medium without phosphate (AD7-Mel P-) prior to inoculation in baffled flasks. Liquid cultures (25 mL total volume, three biological replicates per strain) were grown in 100 mL baffled Erlenmeyer flasks, in a 740-FHC LED incubator (HiPoint Corporation, Taiwan), at 38° C. in air supplemented with 1% (v/v) CO₂, under constant illumination of 300 μmol photons·m⁻²·s⁻¹, using an LED Z4 panel, set to 215 μmol photons·m⁻²·s⁻¹ of red light (660 nm), 50 μmol photons·m⁻²·s⁻¹ of green light (520 nm) and 35 μmol photons·m⁻²·s⁻¹ of blue light (450 nm) and shaken at 200 rpm. Cell growth was monitored by measuring the optical density at 730 nm (OD₇₃₀) in a 1-cm light path with a Cary 300Bio (Varian) spectrophotometer. For dry cell weight determination, 1 to 2 mL culture volume at a determined OD₇₃₀ (between 8 and 10) were filtered onto pre-dried and pre-weighed glass microfiber filters (47 mm diameter, 1 μm pore size, GE Healthcare, Cat. No. 1822-047), washed twice with deionized water and dried overnight at 65° C. (until mass deviation between readings was no higher than 0.0001 g). All measurements were performed using biological triplicates of each strain.

Supercompetent Escherichia coli cells (Stellar, TaKaRa) were used for construction of all relevant plasmids and were cultured in LB medium, at 37° C., supplemented with either 50 μg·mL⁻¹ kanamycin, 50 μg·mL⁻¹ spectinomycin, 50 μg·mL⁻¹ gentamycin or 100 μg·mL⁻¹ carbenicillin, as appropriate. Unless otherwise specified all chemicals utilized were procured from Sigma-Aldrich.

### Strain Construction

### Me/Amine Growing Strain

All PCR reactions were performed using Q5 DNA polymerase (New England Biolabs, NEB) unless otherwise specified and PCR products were routinely digested overnight with Dpnl and purified using a the EZ-10 Spin Column PCR Products Purification Kit (BioBasic) prior to DNA assembly. A DNA fragment comprising the gIpK neutral genomic integration site [Begemann et al., PLoS One. 8: e76594.10.1371/journal.pone. 0076594 (2013)] flanked by 500-bp upstream and downstream regions was PCR amplified from Syn7002 genomic DNA (gDNA) using primers D08807 and D08808 (see Table 1).

TABLE 1
List of primers used

| Name | Sequence | SEQ ID |
|---|---|---|
| D08807 | GCACTGTGGCAAGGAAATCG | 1 |
| D08808 | TCGCCTTTATGGAGGATGGC | 2 |
| D98496993 | TAAAAAAGACTTTATGACTGCTTTACTG | 3 |
| D77036 | GGCTCAAAAGACATCATTTAGG | 4 |
| D98847023 | gaaggttttctgttatctgaaattcctccctaaatgatgtcttttgagccAAAGGTGCTTGTGTCTCAAC | 5 |
| D98847024 | tcatggtgtatatctcctaatcaatTTAGAGCATTTCAAAGTAGGC | 6 |
| D98847025 | tcgggcctactttgaaatgctctaaATTGATTAGGAGATATACACCATG | 7 |
| D98847026 | gggaataatgttggtcatggagcagtaaagcagtcataaagtcttttttaTCGCTCGTCATTTGCTTTC | 8 |
| D99280067 | TGAAGATCAACACCATCTATGACTTAGCC | 9 |
| D99280068 | TAACCCCATGCAAACGCCATC | 10 |
| Mel_seq_1 | TGCCATTGGTATGAGTGAACAAGC | 11 |
| Mel_seq_2 | TCTCTCCGATTGTACCGGTGC | 12 |
| Mel_seq_3 | AATGGATCAAATCTTTTATTTGTGAAGAAAGTGG | 13 |
| Mel_seq_4 | ATGATGTGGCCCGCTCC | 14 |
| Mel_seq_5 | TCGTGGATAAACTCTTTGTGATGATGACC | 15 |
| Mel_seq_6 | CATGCCCAAATTACCGAAACC | 16 |
| Mel_seq_7 | TGCTCTCTGATAGTGATATTAATTCCACC | 17 |
| Mel_seq_8 | GAAGGTGAACCCATTATTGATAAACC | 18 |
| Mel_seq_9 | TCCATCAATTTCAAATTCTCAAGTCTCG | 19 |
| Mel_seq_10 | AGCAATCCCCTCAAAAAATTTAGTTCC | 20 |
| Mel_seq_11 | TCATTGGCAAAAAATTTACCGATTATGC | 21 |
| Mel_seq_12 | TGCCCAATGTGACCGTGG | 22 |
| Mel_seq_13 | TCAAAGAAGAATACCCCGATACCTG | 23 |
| Mel_seq_14 | TCGATGAACGCCTCCAAGTGG | 24 |
| D100023580 | ACCCGGGGATCCTCT AGAGCCAGATCCTTTTGCATC | 25 |
| D100023581 | CTGCAGGTCGACTCT TGAAGGGAGCCAGAACATAAAAAG | 26 |
| A0935_UCO_F | TGACTTGGTTCACGT AGAAAAACCAGAAGGGAC | 27 |
| A0936_UCO_R | TGTCCACTCCTTAAT CACTATTCAAAATATTATATTTACTCAGTTTTTAAG | 28 |
| D98496996 | ATTAAGGAGTGGACAGCCTAC | 29 |
| D100141467 | ACGTGAACCAAGTCAGACAATC | 30 |
| D100043610 | TAGATGATGATGTAGAATTGTCTGCTAATTAC | 31 |
| D100043611 | TAATGATTTCAGTACAAATTGCTCTGC | 32 |
| D100263687 | tttgattgtctgacttggttcacgtTTGAGGCCGTTGATCTAGACAAAAAAC | 33 |
| D100263688 | aggagattaattccatgggccatc | 34 |
| D100098818 | AGAAAAACCAGAAGGGACG | 35 |
| D101108991 | taaagtcaagtagAagattaattcc ATGGGCCATCATCATCATCATCATCATC | 36 |
| D101108992 | ggaattaatctTctacttgacttta TGAGTTGGGAGCTCCTTTTGCAATC | 37 |
| D74727 | TTCACAGAGGAAGGGGAAATTGTC | 38 |
| D74729 | TCATATCCGGGGCATACATTCG | 39 |
| D101108989 | TTAAgctagttagAagattcagacc ATGCAAACCCTCAGCATTCAACATG | 40 |
| D101108990 | ggtctgaatctTctaactagcTTAA TTCCGTTCGTACACGAGGCGAC | 41 |
| D15106 | gttgtaaaacgacggccagtgaatt TTAAGCCTGGGGTGAGTTGAC | 42 |
| D15107 | CGTTTccagtTGTCCACTCCTTAAT TAATTTCTCAAGGGAAAGAAAAAGATTTATTCc | 43 |
| D15108 | ttaaaaagcaTGACTTGGTTCACGT ACGAAGGTTGTTTTTAAAGCTAAGAAG | 44 |
| D15109 | caggaaacagctatgaccatgatta TATGAAAATGCTTCACACCATGATTCG | 45 |
| D15110 | gttgtaaaacgacggccagtgaatt ACAATTGACACAAAAAAGAGAGCAAAG | 46 |
| D15111 | GCTCCGGCTTTGACTTGGTTCACGT GTGACCCTCTTGCAGCAACC | 47 |
| D15112 | TTCCACGGTGTGTCCACTCCTTAAT TTGCCTGATTATGCTTCCATCAAAATTTG | 48 |
| D15113 | caggaaacagctatgaccatgatta TATCCGTCACGGTGGCTC | 49 |
| D39394 | TAGGTAGTATTGGGGCATCAGG | 50 |
| D39395 | TAATCTCGATGTGATGATTGCTGAAGG | 51 |
| D99047654 | ATTAAGGAGTGGACA CACCGTGGAAACGGATGAAG | 52 |
| D99047655 | ACGTGAACCAAGTCA AAGCCGGAGCGCTTTTG | 53 |
| D98646038 | ATTAAGGAGTGGACA ACTGGAAACGGATGAAGG | 54 |
| D98646039 | ACGTGAACCAAGTCA TGCTTTTTAAGGGAATTGTGC | 55 |
| RF_ptxD_F | GGCGCCACCCTGCAATATCACGCTCGTAAAGCGCT | 95 |
| RF_ptxD_R | CCGTTTGCGTATCCAGCGCTTTACGAGCGTGATA | 96 |

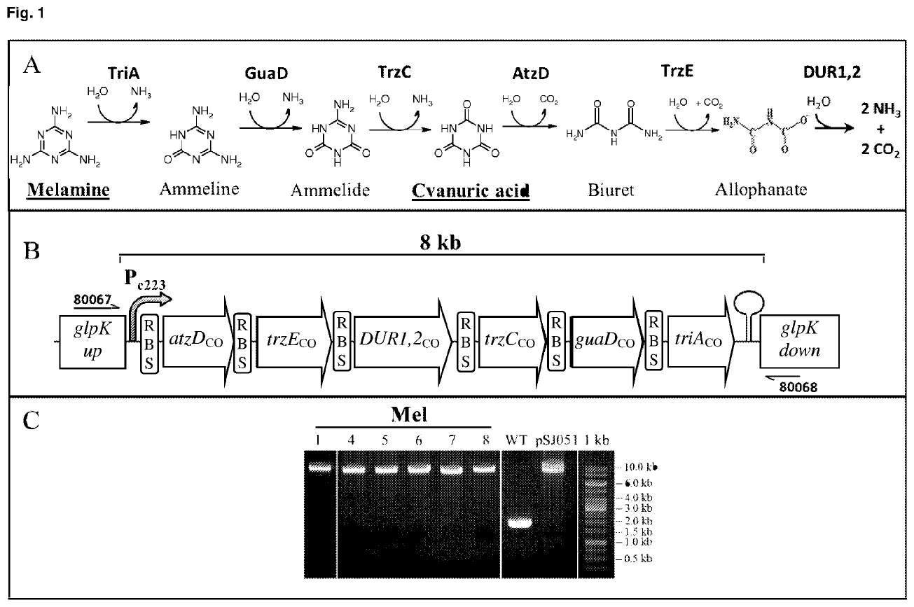

The purified PCR product was ligated into pCR-Blunt II TOPO (Invitrogen) following the manufacturer's instructions and transformed into chemically competent Stellar E. coli cells, resulting in plasmid pCRBlunt-glpK (correct assembly was confirmed by Sanger sequencing using universal M13 primers). Using primers D98496993 and D77036, the pCRBlunt-glpK backbone was reverse-PCR amplified, and the melamine operon was amplified in two halves from a synthetic construct (GenScript, Hong Kong, Ltd.) using primers D98847023 and D98847024 (first half) and D98847025 and D98847026 (second half). Both fragments were assembled into pCRBlunt-glpK using the NEBuilder HiFi DNA Assembly Master Mix (NEB), as per the manufacturer's instructions. 1 μL of the assembly mix was transformed into Stellar E. coli supercompetent cells, resulting in plasmid pSJ051. Correct assembly of the melamine operon was confirmed by Sanger sequencing using the primers Mel_seq_1 to Mel_seq_14, D99280067 and D99280068, indicated in Table 1. Syn7002 WT was transformed by double homologous recombination as previously described [Frigaard et al., Methods Mol Biol. 274: 325-40 (2004)], with modifications. Briefly, 2 μg of pSJ051 were used to transform 2 mL of a Syn7002 culture at an OD₇₃₀ of 0.5 and incubated overnight, as described above, in a 12 mL round bottom snap-cap tube. The following day the culture was spun down, the pellet resuspended in 50 μL of the supernatant prior to spreading on an AD7-Cya plate, to favour integration of the entire melamine operon into the glpK site. This plate was incubated under the conditions described above until colonies appeared (after 2 weeks). Eight colonies were picked and re-streaked 4 times on AD7-Cya plates and, subsequently, on AD7-Mel plates until full chromosomal segregation, tested using primers D99280067 and D99280068 (see FIG. 1), was confirmed. Only six of the initial eight colonies survived, having evolved into strains Mel1, Mel4, Mel5, Mel6, Mel7 and Mel8.

### Phosphite-Utilizing and Combined Melamine/Phosphite-Utilizing Strains

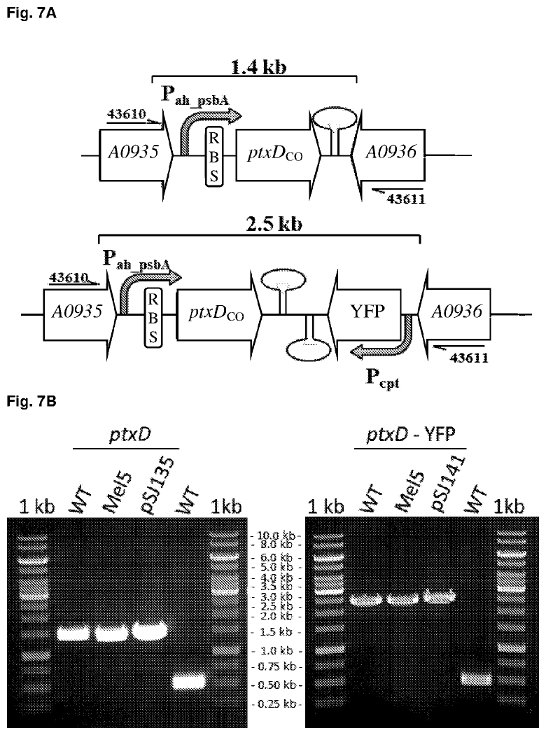

A region 500 bp upstream to 500 bp downstream of the neutral genomic integration site between ORF A0935 and A0936 [Davies et al., Frontiers in bioengineering and biotechnology. 2: 21.10.3389/fbioe.2014.00021 (2014)] was PCR amplified from Syn7002 gDNA using primers D100023580 and D100023581. pUC19 (Invitrogen) was digested with Xbal (NEB) and purified from the agarose gel band using the EZ-10 Spin Column DNA Gel Extraction Kit (BioBasic). The A0935-A0936 site was assembled into the digested pUC19 backbone using the pEASY-Uni Seamless Cloning and Assembly Kit (TransGen Biotech Co., Ltd, China), according to the manufacturer's instructions, and transformed into Stellar E. coli cells, resulting in plasmid pSZT001. Primer pair A0935_UCO_F and A0936_UCO_R were used to reverse-PCR amplify the pSZT001 backbone and pair D98496996 and D100141467 to amplify a synthetic, codon-optimized version (SEQ ID NO: 90) (by GenScript, Hong Kong, Ltd) of the Pseudomonas stutzeri WM88 phosphite dehydrogenase (ptxD) gene [Loera-Quezada et al., Plant Biotechnol J. 14: 2066 (2016)], driven by the Amaranthus hybridus constitutive psbA promoter (SEQ ID NO: 92) [Elhai and Wolk, Gene. 68: 119-138 (1988)]. Both fragments were assembled using the pEASY-Uni kit, as described above, resulting in plasmid pSJ135. 2 μg of this plasmid were used to transform both Syn7002 WT as well as the Mel5 strain, as described above, with the exception that, prior to transformation, cultures were spun down and washed twice with AD7 medium lacking phosphate (AD7-NO₃ P- for WT and AD7-Mel P- for Mel5) and subsequently plated onto either AD7-NO₃ Phi 1× (0.370 mM phosphite) or AD7-Mel Phi 1×, respectively. Both plates were incubated under the conditions described above until colonies appeared (10 days). Eight colonies from each plate were picked and consecutively re-streaked on AD7-NO₃ Phi 1× or AD7-Mel Phi 1× until full chromosomal segregation was confirmed by colony PCR using primers D100043610 and D100043611 (see FIG. 7), resulting in strains A0935-ptxD and Mel5-ptxD, respectively.

To further test the use of phosphite as a selectable marker, a DNA fragment containing an YFP gene under the control of the strong constitutive P_{cpt} promoter [Markley et al., ACS Synth Biol. 4: 595 (2015)] was amplified from pAcsA-cpt-YFP (a kind gift from Prof. Brian Pfleger, University of Wisconsin-Madison, USA) using primers D100263687 and D100263688. The pSJ135 backbone was reverse-PCR amplified using primers D100141467 and D100098818 and the two fragments assembled using the pEASY-Uni kit, yielding pSJ141. Transformation of either Syn7002 WT or Mel5, using phosphite media for selection of transformants, and selection of fully segregated strains by colony PCR was performed as described above.

### Knock-Out of Putative Phosphonate Transporters

Putative phosphonate transporter genes were identified by using the BlastP tool within CyanoBase (http://genomedotmicrobedbdotjp/blast/blast_search/cyanobase/genes), limiting the search to Syn7002 and using as a search template the amino acid sequence of PhnD from Prochlorococcus marinus sp. MIT9301 [Bisson et al., Nat Commun. 8: 1746.10.1038/s41467-017-01226-8 (2017); Feingersch et al., ISME J. 6: 827-34 (2012)]. Two homologues (annotated as putative phosphate/phosphonate-binding ABC transporters) were identified in Syn7002: A0336 (E value=1e⁻⁹⁶) and G0143 (E value=2e⁻⁰⁸). DNA sequences 500 up- and downstream of these genes were amplified from Syn7002 cells using primers D15106 and D15107 (A0336 upstream region), D15108 and D15109 (A0336 downstream region), D15110 and D15111 (G0143 upstream region) and D15112 and D15113 (G0143 downstream region). Purified DNA fragments were assembled into an Xbal-digested pUC19 fragment with either a spectinomycin-resistance cassette (in the case of A0336), amplified from pBAD42 (using primers D98646038 and D98646039) or a gentamycin-resistance cassette (in the case of G0143), amplified from pVZ322 (using primers D99047654 and D99047655), using the NEBuilder HiFi DNA Assembly Master Mix, according to the manufacturer's instructions, resulting in pSJ156 (pUC19-ΔA0336::SpR) and pSJ157 (pUC19-ΔG0143::GmR). Both plasmids were used to transform Syn7002 WT and the A0935-ptxD strain and, upon full segregation, pSJ157 was used to transform ΔA0336::SpR deletion strains in the WT (WTΔA0336 strain) and A0935-ptxD (ptxDΔA0336 strain) backgrounds, resulting in double-knockout strains WTΔA0336ΔG0143 and ptxDΔA0336ΔG0143. WT Syn7002, A0935-ptxD, ptxDΔA0336, ptxDΔD0143 (ΔG0143::GmR in an A0935-ptxD background) and ptxDΔA0336ΔG0143 were grown in regular ΔD7 (Pho 1×) medium, in the presence of appropriate antibiotic concentrations (50 μg·mL⁻¹ spectinomycin and/or 50 μg·mL⁻¹ gentamycin), under the conditions described above. Cultures of all strains were washed twice with AD7-NO₃ P-, resuspended to an OD₇₃₀=4 in the same medium and serially diluted 1:10 (from 4×10⁰ to 4×10⁻⁵) using the same AD7-NO₃ P-medium. 10 μL of each dilution were spotted on either AD7-NO₃ Pho 1× or AD7-NO₃ Phi 20×, cultured under the conditions described above, for 5 days.

### Yellow Fluorescent Protein (YFP) Fluorescence Measurement

Whole cell YFP fluorescence was determined for triplicate cultures (15 mL each) grown in regular AD7 medium to an OD₇₃₀ between 0.5 and 1, with 150 μL aliquots measured using 96-well black clear bottom plates, in a Hidex Sense (excitation: 485/10 nm; emission: 535/20 nm). Fluorescence was measured in triplicates for each culture and normalized to OD₇₃₀ (measured at the same time by the Hidex Sense plate reader, as described above), using AD7 medium as blank control.

### Genome Sequencing

Genomic DNA was prepared from both the WT strain as well as the different melamine-utilizing strains by using the Quick-DNA Fungal/Bacterial Kit (Zymo Research). Library preparation was performed according to Illumina's TruSeq Nano DNA Sample Preparation protocol. The samples were sheared on a Covaris E220 to ˜550 bp, following the manufacturer's recommendation, and uniquely tagged with one of Illumina's TruSeq LT DNA barcodes to enable sample pooling for sequencing. Finished libraries were quantitated using Promega's QuantiFluor dsDNA assay and the average library size was determined on an Agilent Tapestation 4200. Library concentrations were then normalized to 4 nM and validated by qPCR on a QuantStudio-3 real-time PCR system (Applied Biosystems), using the Kapa library quantification kit for Illumina platforms (Kapa Biosystems). The libraries were then pooled at equimolar concentrations and sequenced on the Illumina MiSeq platform at a read-length of 300 bp paired-end. Genomes were assembled and compared using the Geneious 11.1.4 software (Biomatters Ltd.).

### Ribosome Binding Site (RBS) Point Mutation Test

To assess the effect of the RBS change in Mel5, the original pSJ051 plasmid was mutated at the RBS upstream of triA (changed from AGGAGA to AGAAGA) by inverse PCR [Liu and Naismith, BMC Biotechnol. 8: 91.10.1186/1472-6750-8-91 (2008)] using Q5 DNA polymerase (NEB) using primers D101108989 and D101108990, resulting in plasmid pSJ155. This plasmid was used to transform Syn7002 WT, using AD7-Cya and AD7-Mel plates, as described above. The resulting strain, Re-Mel5, was re-streaked twice on AD7-Mel plates and tested for growth in AD7-Mel as described above.

### Co-Culturing Competition Experiments and Flow Cytometry

Growth competition experiments were performed by culturing a Syn7002 strain transformed with the pAcsA-cpt-YFP plasmid (constitutively expressing YFP, termed “cptYFP”) and Mel5-ptxD in either AD7-NO₃ 1×Pho or AD7-Mel 20×Phi. Strains were diluted to a starting OD₇₃₀ of 0.05 and their growth (in biological triplicate cultures) followed by flow cytometry using a 3-laser BD LSR Fortessa X20, using the channels of FITC (ext: 488 nm; em: 525/50 nm) to detect YFP-positive cells and APC (ext: 633 nm; em: 670/30 nm) for Chlorophyll a (ChI a)-positive cells. For cell counting, cultures were first diluted to an OD₇₃₀ of roughly 0.05 as needed and were acquired on the Fortessa X20 flow cytometer at a consistent rate of 3000 events/s. A log-scale plot of forward scatter (on the x-axis) vs side scatter (on the y-axis) was used as the initial gating to select for live cells and then analysed for ChI a-positive cells. This was then used to draw gates for ChI a-only (Mel5-ptxD) or double positive ChI a/YFP (cptYFP) cells. Cell counts were obtained by acquiring to exhaustion a set sample volume of 50 μL. Cell counts in triplicate samples were derived using the BD FACS Diva Software (v. 8.0) and back-calculated based on the dilution utilized.

### Identification of Melamine Pathway Intermediates Using LC-MS/MS

Cultures of Syn7002 and melamine-utilizing strains were collected after 48 hours of growth and spun down (14000 g, 5 min, room temperature). Supernatants were filtered through 0.2 μm syringe filters (Acrodisc filters with Supor membrane, PALL) and frozen at −80° C. until further use. Melamine, ammeline, ammelide and cyanuric acid were quantified by LC-MS/MS using a previously described method [Braekevelt et al., Food Addit Contam Part A Chem Anal Control Expo Risk Assess. 28: 698-704 (2011)] at the NTU Phenomics Centre.

### Example 2

### Introduction of Melamine Degradation Pathway Requires Evolutionary Adaptation for Efficient Usage

The melamine degradation pathway utilized in this study is based on the optimized pathway (genes triA, guaD, trzC, atzD, trzE and DUR1,2 including the described R352S mutation in the guaD gene product) reported by Shaw and co-workers [Shaw et al., Science. 353: 583-6 (2016)] (FIG. 1). In our case we used codon-optimized genes (triA, SEQ ID NO: 70; guaD, SEQ ID NO: 80; trzC, SEQ ID NO: 74; atzD, SEQ ID NO: 78; trzE SEQ ID NO: 72; and DUR1,2, SEQ ID NO: 76), a synthetic strong cyanobacterial promoter P_{c223} (SEQ ID NO: 82)[Markley et al., ACS Synth Biol. 4: 595 (2015)] and the strongest RBS sequence (AGGAGA) tested in Syn7002 [Markley et al., ACS Synth Biol. 4: 595 (2015)] upstream of all 6 genes. Intergenic regions (21 bp in total), including spacers before (7 bp) and after the RBS sequence (8 bp), were generated by a random DNA sequence generator (http://worldwidewebdotfacultydotucrdotedu/˜mmaduro/randomdothtm) and a vector was constructed to target the entire pathway to the glpK neutral site [Begemann et al., PLoS One. 8: e76594.10.1371/journal.pone.0076594 (2013)] of WT Syn7002 (see Example 1 for details). To avoid introducing an antibiotic cassette, transformants were selected on plates containing 2 mM melamine (AD7-Mel plates). However, despite several attempts, it was not possible to obtain colonies when plating directly onto AD7-Mel plates (data not shown). We hypothesised that positive transformants could be selected for by plating cells onto AD7 plates containing 4 mM cyanuric acid rather than melamine as cyanuric acid is an intermediate in melamine degradation, formed in the 3^{rd} step of the pathway (FIG. 1A). This strategy led to the isolation of a small number of cyanuric acid growing colonies. However, these colonies were initially unable to grow on AD7-Mel plates and were re-streaked 4 times on AD7-Cya plates before growth could be finally achieved on AD7-Mel plates. Six isolated colonies (designated Mel1, Mel4, Mel5, Mel6, Mel7 and Mel8) grown on AD7-Mel plates were analysed further and shown to contain the entire Mel operon (FIG. 1B) and to be fully segregated as judged by PCR analysis (FIG. 1C). The polynucleotide sequences of the Mel1, Mel4, Mel5, Mel6, Mel7 and Mel8 operons are set forth in SEQ ID NO: 83, 84, 85, 86, 87 and 88, respectively.

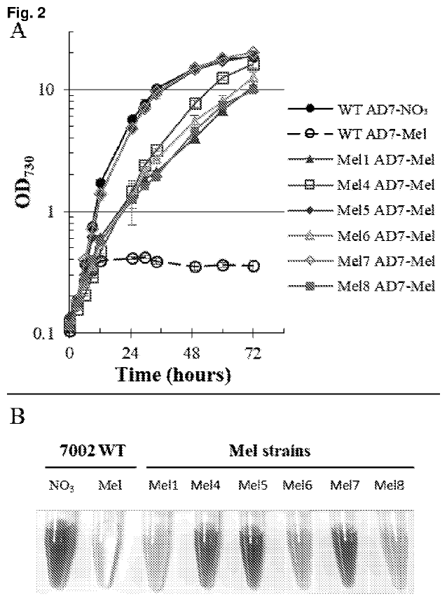

The growth of the different Mel strains and WT Syn 7002 in either AD7-Mel medium (containing 2 mM melamine) or regular AD7 medium (containing nitrate) were compared. As can be seen in FIGS. 2A and B, the different individual strains, unlike the parental Syn7002 WT strain, were able to grow on melamine as sole nitrogen source, albeit at different rates. Two strains in particular, Mel5 and Mel7, could grow almost as well in AD7-Mel as Syn7002 WT in AD7-NO₃ while the remaining strains had slower growth and a different colouration (FIG. 2B), a common indication of stress [Collier and Grossman, Journal of Bacteriology. 174: 4718-26 (1992)].

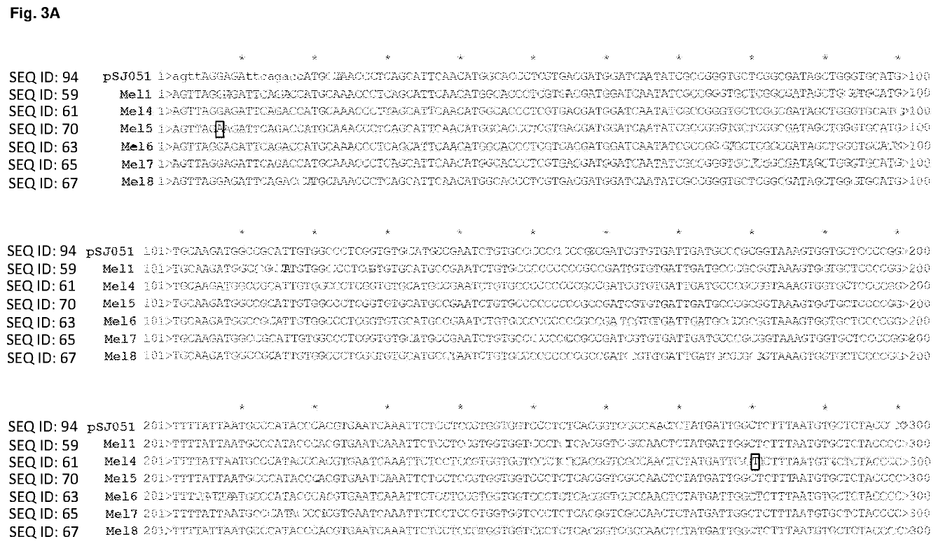

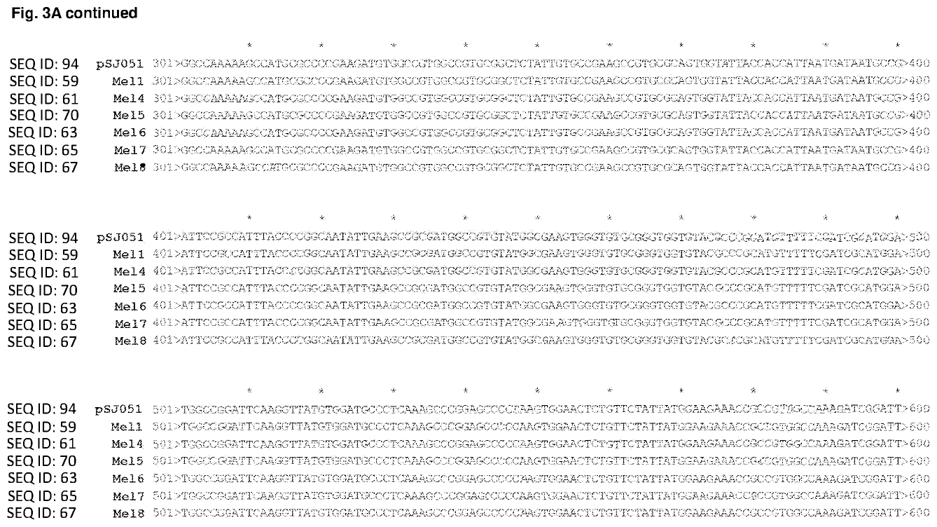

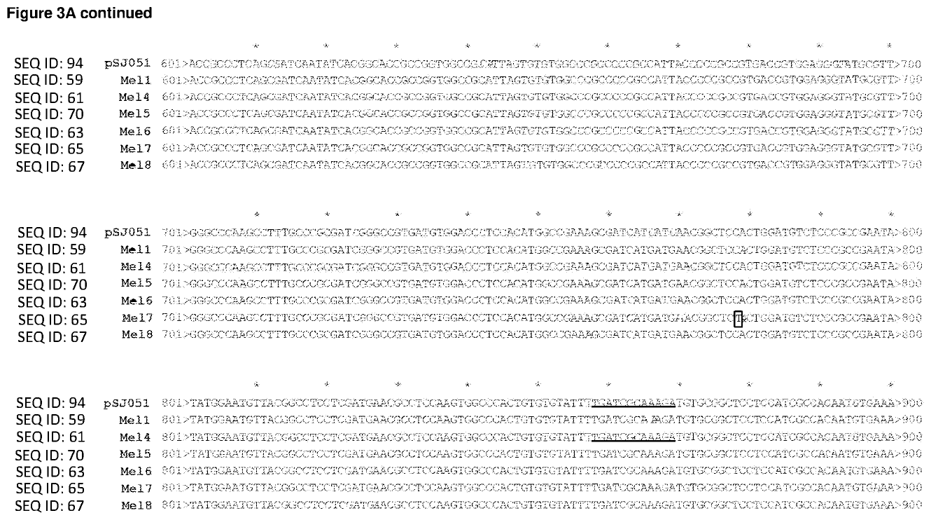

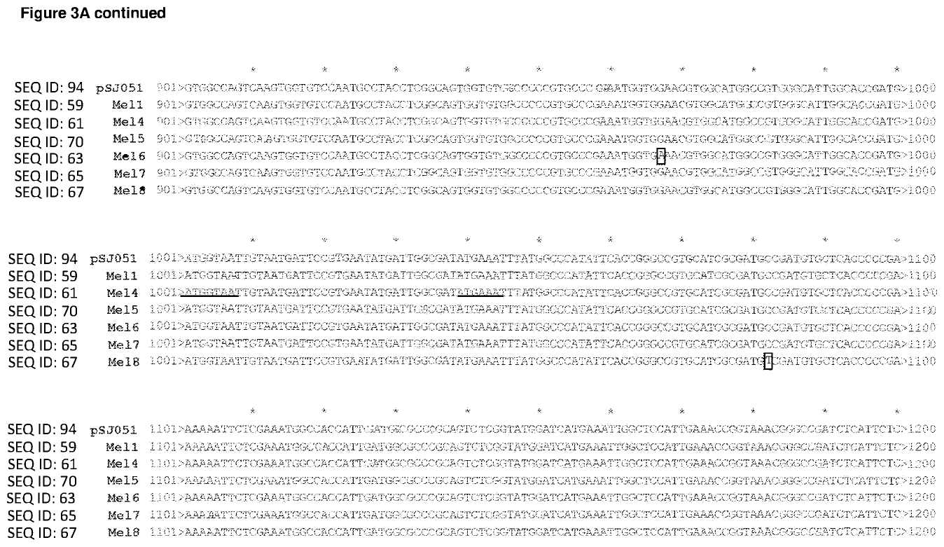

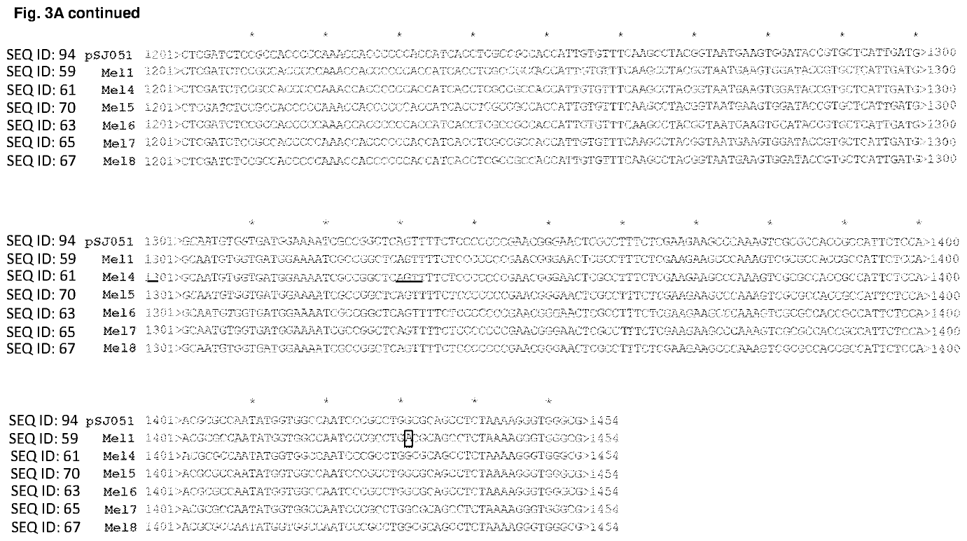

To further understand the reasons behind these varying phenotypes we sequenced the genomes of all the melamine-growing strains (Mel1, Mel4, Mel5, Mel6, Mel7 and Mel8) as well as of the Syn7002 WT used in this experiment (obtained from the laboratory of Prof. Donald Bryant, Penn State University, USA). Comparison of the sequences pinpointed several mutations in the melamine operon, all of them located either in the RBS preceding the triA (encoding melamine deaminase) gene or within the triA gene itself (FIG. 3A). The mutations in the triA amino acid sequence were at positions Leu88Phe (Mel4), His254Tyr (Mel7), Glu317Lys (Mel6), Ala355Val (Mel8) and Trp471stop (Mel1). Mel5 had a mutation in the RBS (AGGAGA to AGAAGA) (FIG. 3B). As this is the first step in the melamine degradation pathway, mutations affecting triA or its RBS will regulate metabolic flux through the rest of the pathway.

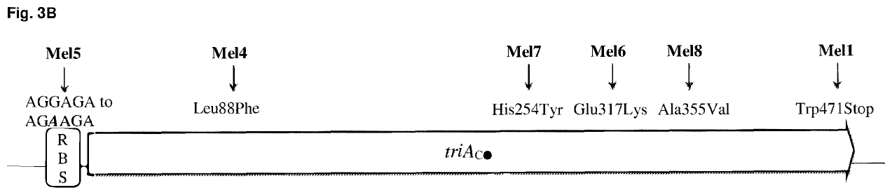

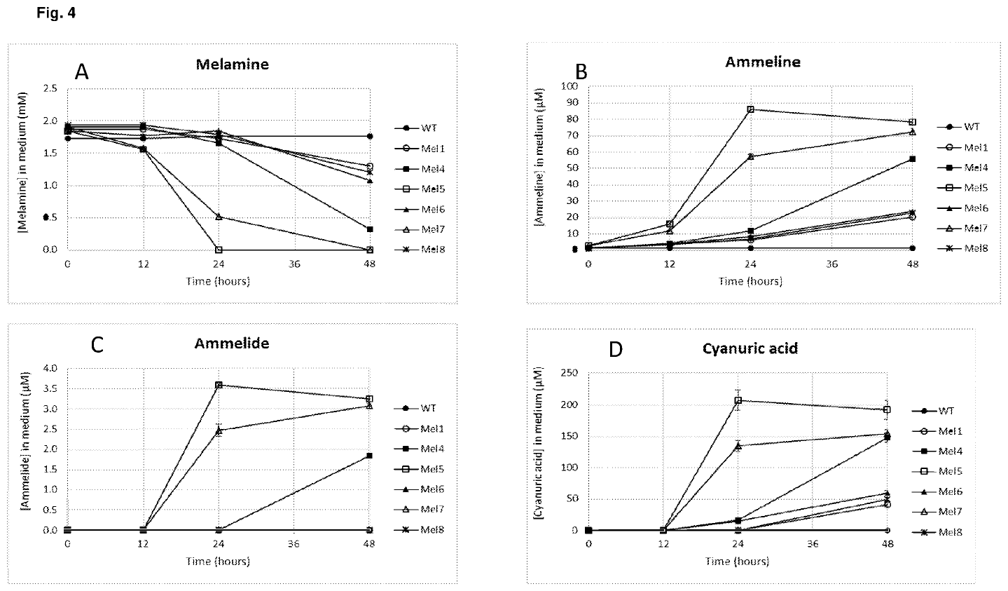

To further clarify the changes occurring in the melamine pathway's metabolic flux in the different strains, LC-MS/MS was used to quantify the pathway intermediates excreted into the growth medium from melamine to cyanuric acid (FIG. 4). Melamine was very rapidly consumed, within the first 24 hours of growth, in both Mel5 and Mel7, at the same time ammeline (the first intermediate after melamine) was found to accumulate significantly more in Mel5 and Mel7 (86.2±1.6 μM for Mel5 and 57.2±1.5 μM for Mel7) than in the remaining strains (525 μM), within the same time frame. Ammelide (the third intermediate) could only be quantified at very low levels (<4 μM detected in Mel5 growth medium after 24 hours) whereas cyanuric acid rapidly accumulated to a concentration of 207.5±16.3 μM in Mel5 and 134.5±8.8 μM in Mel 7 (FIG. 4A-D). The substantial excretion of cyanuric acid into the medium was also reported in the original article on the introduction of the melamine degradation pathway into E. coli at levels of 13% of the initial (molar) amount of added melamine [Shaw et al., Science. 353: 583-6 (2016)], strikingly similar to the value of 9.7% observed in the Mel5 strain after 24 hours. It is likely that the mutations present in the remaining strains do not confer as strong an advantage as those found for Mel5 and Mel7, resulting in low intracellular nitrogen levels and poorer growth rates observed under normal light conditions (FIG. 2). It should be noted that pre-cultures, grown under low light, were much less affected, possibly due to a general metabolic rate slowdown under those conditions (data not shown).

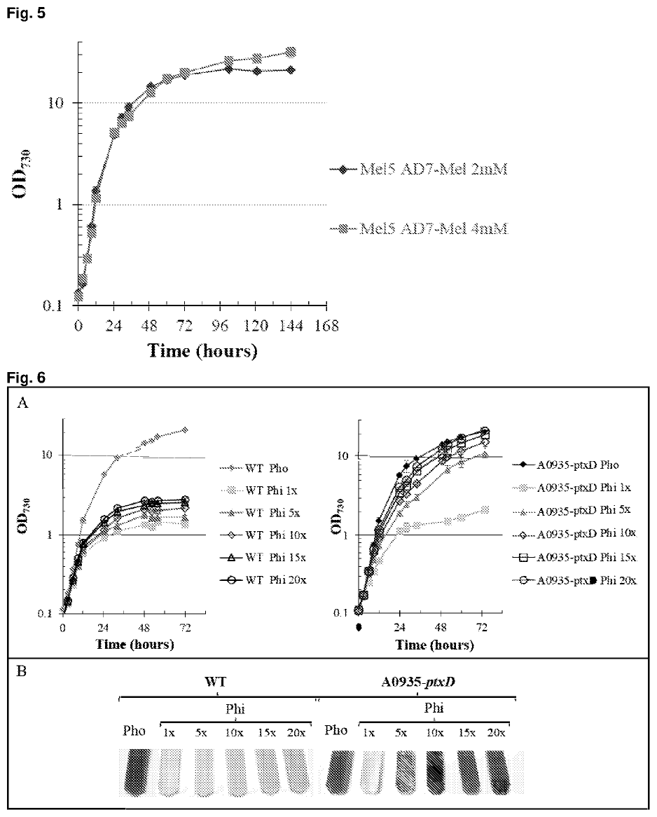

Mel5 was grown in 2 mM and 4 mM melamine and growth rates compared. FIG. 5 shows that after about 72 hours 4 mM melamine can sustain Mel5 to a higher OD than 2 mM melamine.

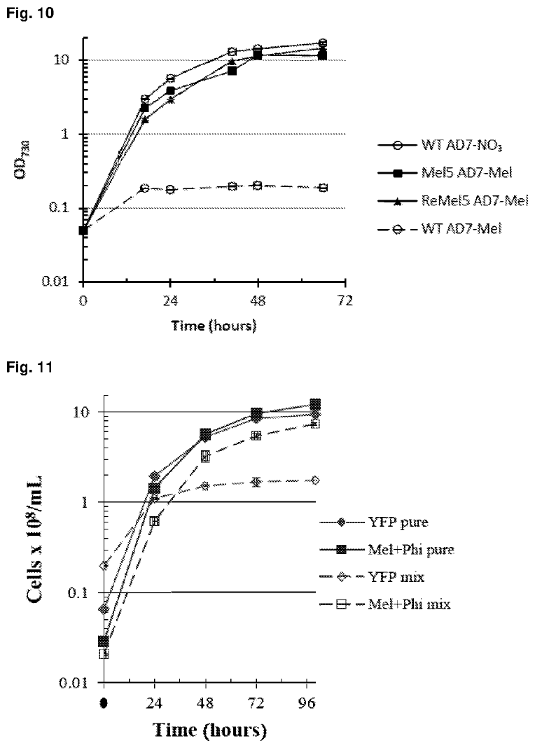

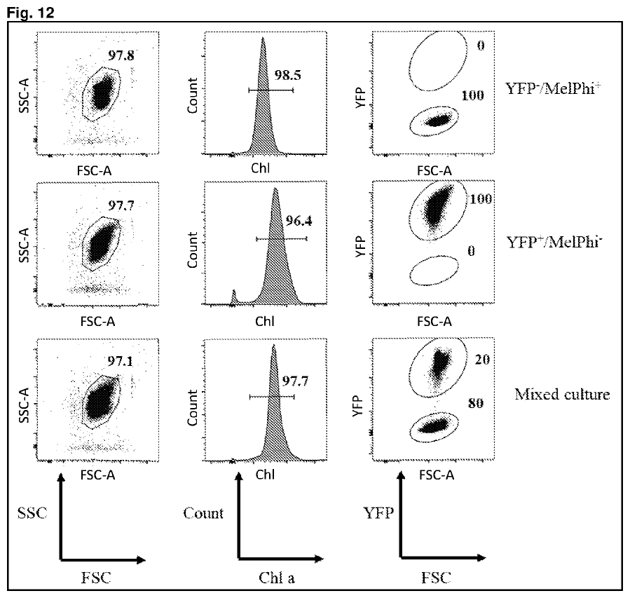

Finally, the RBS sequence was mutated upstream of the triA gene in the original pSJ051 to match the mutation found in Mel5. Using this modified construct colonies were obtained when plated directly on AD7-Mel plates (as well as by plating on AD7-Cya plates). Growth of the newly obtained strain (named “Re-Mel5”) was compared to Mel5 and Re-Mel5 was found to reach a similar OD₇₃₀ after 48 hours when grown on melamine as the sole nitrogen source (FIG. 10), indicating that the mutation found was indeed able to improve melamine utilization efficiency.

### Example 3

Phosphite and PtxD can be Used as an Efficient Selection System in Synechococcus sp. PCC7002

Though phosphite (Phi) was previously shown to be able to sustain growth of modified strains of both Synechocystis sp. PCC 6803 [Polyviou et al., Environmental microbiology reports. 7: 824-30 (2015)] and Synechococcus sp. PCC 7942 [Motomura et al., ACS Synth Biol.10:1021 (2018)], in both cases the genetic manipulations (integration of the operon containing both ptxD as well as a specific Phi transporter) were driven by antibiotic selection pressure. At the same time neither of the two strains (wild-type Synechocystis sp. PCC 6803 nor wild-type Synechococcus sp. PCC 7942) seemed to be able to take up Phi without inclusion of a specific transporter, thus making the construct too large to be of practical use as a selectable marker. As no data exist in the literature regarding the ability of Syn7002 to grow on Phi as sole P source, growth of the WT strain was tested using varying concentrations of Phi (FIG. 6A, left panel). Though there was some growth for the first 24-36 hours, no observable growth occurred after this period, which was probably due to the full consumption of internal phosphate reserves—cyanobacteria store phosphorus as polyphosphate granules in the cytoplasm and have a dynamic mobilization mechanism that allows them to tap into these reserves when needed [Gomez-Garcia et al., Journal of bacteriology. 195, 3309 (2013)].

Syn7002 WT transformed with a construct (pSJ135) containing the Pseudomonas stutzeri WM88 phosphite dehydrogenase gene (codon optimized; SEQ ID NO: 90) in the neutral site A0935 and no other selectable marker (FIG. 7A, top) was plated on AD7 plates with Phi (0.37 mM, 1×) as sole phosphorus source. This transformation yielded many hundred colonies, and, though the transformants (A0935-ptxD) had a yellowish tinge, characteristic of phosphate-starved cells, this concentration of Phi was sufficient to induce full chromosomal segregation of the transformed strains (FIG. 7B), thus validating the ptxD gene and phosphite as a valid selection strategy in Syn7002.

Given that the ptxD gene alone was enough to permit growth of strain A0935-ptxD on Phi, it would seem that, unlike the related (freshwater) strains Synechocystis sp. PCC6803 and Synechococcus sp. PCC7942, Syn7002 is able to import Phi from the growth medium, through a so far undefined transporter.

To test whether a higher concentration gradient would be sufficient to enhance transport into the cells and allow faster growth on Phi, growth of A0935-ptxD was tested in AD7 with increasing Phi concentrations (from 0.37 mM to 7.4 mM). As can be seen in FIG. 6A (right side) the highest Phi concentration tested (Phi 20×, 7.4 mM) allowed this strain to attain growth rates nearing those of Pho-grown cells. At the same time, the relation between grams dry cell weight (gDCW) and OD₇₃₀ also increased with increasing Phi concentrations, from 0.1633±0.014 gDCW·L⁻¹·OD₇₃₀⁻¹ for cells grown in AD7-Phi 1× to 0.2038±0.003 gDCW·L⁻¹·OD₇₃₀⁻¹ for cells grown in AD7-Phi 20× (Table 2), a value similar to that of WT cells grown in standard conditions (0.2145±0.007 gDCW·L⁻¹·OD₇₃₀⁻¹). This increase might be due to the more efficient Phi uptake and conversion at higher Phi concentrations.

TABLE 2
Calculated ratios between OD₇₃₀ and grams dry
cell weight (gDCW)/L for each strain tested.

| gDCW L⁻¹ OD₇₃₀⁻¹ | | |
|---|---|---|
| Strain and condition | Average | St Dev |
| WT AD7-NO₃ | 0.2145 | 0.007 |
| Mel 1 AD7-Mel | 0.2371 | 0.022 |
| Mel 4 AD7-Mel | 0.2181 | 0.007 |
| Mel 5 AD7-Mel 2 mM | 0.2083 | 0.002 |
| Mel 6 AD7-Mel | 0.2293 | 0.005 |
| Mel 7 AD7-Mel | 0.2079 | 0.012 |
| Mel 8 AD7-Mel | 0.2451 | 0.009 |
| Mel 5 AD7-Mel 4 mM | 0.2222 | 0.006 |
| A0935-ptxD AD7-Pho 1× | 0.1957 | 0.006 |
| A0935-ptxD AD7-Phi 1× | 0.1633 | 0.014 |
| A0935-ptxD AD7-Phi 5× | 0.1832 | 0.01 |
| A0935-ptxD AD7-Phi 10× | 0.1744 | 0.01 |
| A0935-ptxD AD7-Phi 15× | 0.1942 | 0.008 |
| A0935-ptxD AD7-Phi 20× | 0.2038 | 0.003 |
| MelPhi AD7-NO₃ Pho | 0.2101 | 0.03 |
| MelPhi AD7-NO₃ Phi 20× | 0.2191 | 0.019 |

Note:
Figures are averages and standard deviations from dry weight determination using biological triplicate samples.

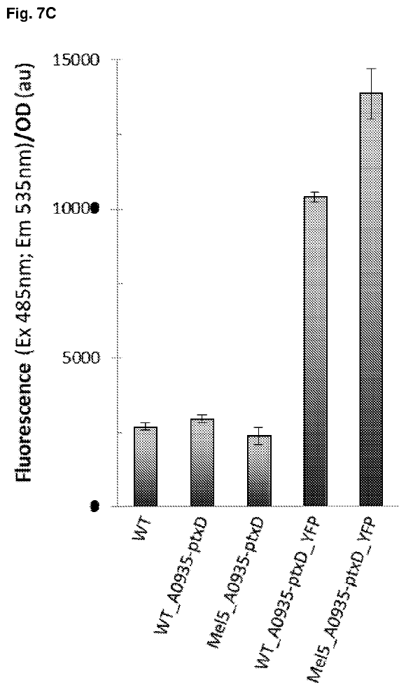

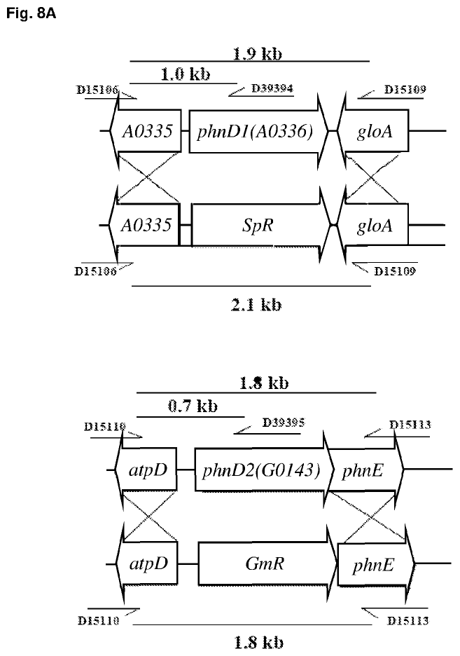

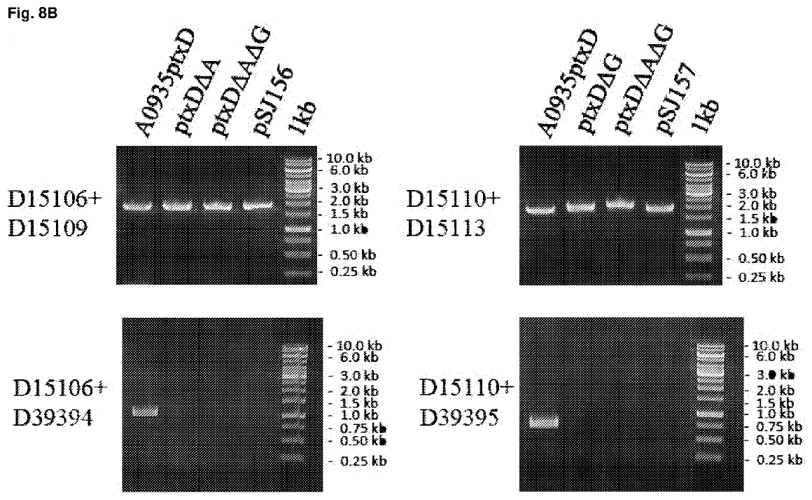

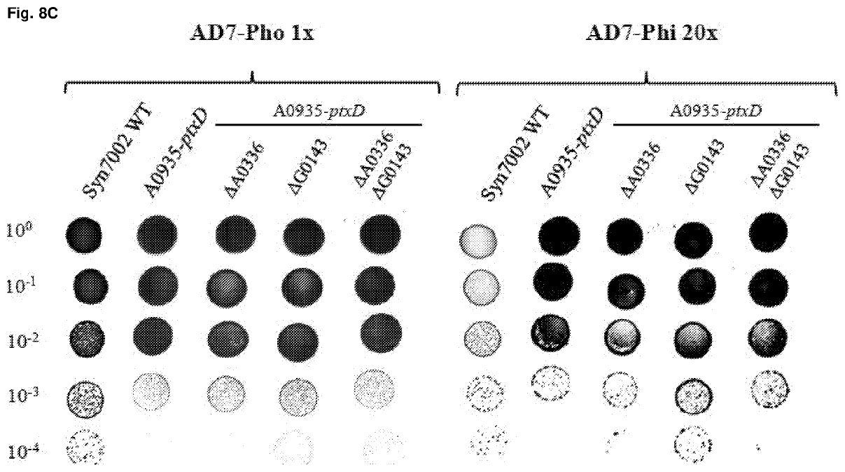

It is known that transgenic Arabidopsis thaliana plants expressing the same ptxD gene (using phosphinothricin for selection) are able to grow using Phi as the sole phosphorus source [Lopez-Arredondo and Herrera-Estrella, Nat Biotechnol. 30: 889 (2012)]. While the specific transporter by which Phi is taken up by plant roots is not known, it would seem that no extra Phi transporter genes are required, as is the case for Syn7002. However, Phi uptake in Syn7002, unlike A. thaliana, is much less efficient. As previous studies have shown that several marine cyanobacteria are able to take up and utilize Phi as a phosphorus source [Feingersch et al., ISME J. 6: 827-34 (2012); Martinez et al., Environ Microbiol. 14: 1363 (2012); Polyviou et al., Environmental microbiology reports. 7: 824-30 (2015)] we searched the Syn7002 genome for putative transporter genes, such as ptxB and phnD homologues [Bisson et al., Nat Commun. 8: 1746.10.1038/s41467-017-01226-8 (2017)]. While no ptxB homologues could be found, two putative phnD genes, A0336 and G0143, were present in either the circular chromosome (A0336) or the pAQ7 plasmid (G0143) (data not shown). We hypothesized that either of these two genes might be involved in phosphite import to the cell, as phnD homologues were shown to also bind phosphite [Bisson et al., Nat Commun. 8: 1746.10.1038/s41467-017-01226-8 (2017)]. However, knock-out of these putative phosphonate transporters, either alone or in combination, did not prevent the ptxD parental strain from grow on phosphite (FIGS. 8A-8C).

To investigate whether this selection method would be sufficient to allow co-integration of other genes, a second construct was designed where the yfp gene would be integrated into the chromosome at the same locus, using ptxD as the selectable marker and Phi as positive selection. As can be seen in FIGS. 7B and 7C, the yfp gene was successfully co-integrated using this method and YFP fluorescence could be measured in positive transformants. Thus, this method can be used to select for integration and expression of heterologous genes in Syn7002.

### Example 4

### Construction of a Strain Capable of Growing on Both Melamine and Phosphite

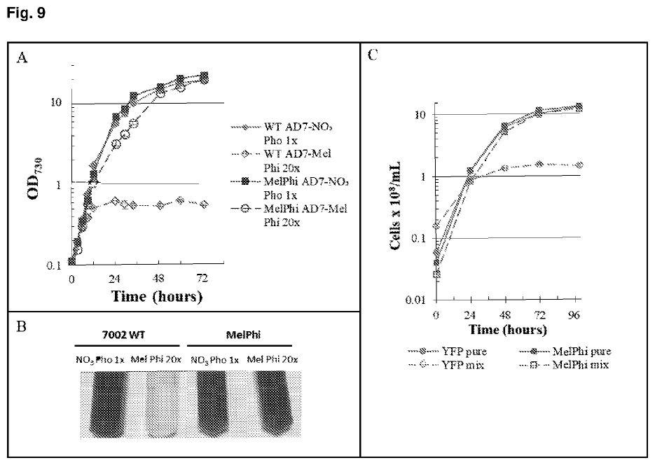

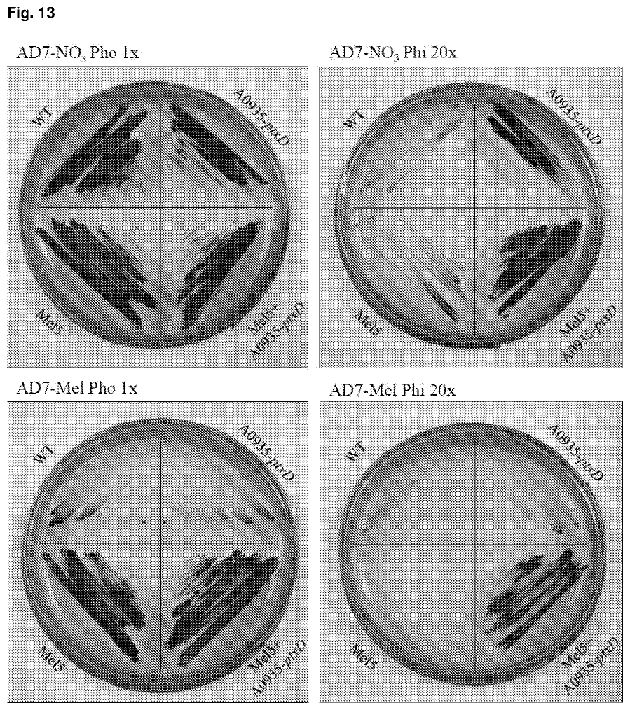

The above examples demonstrate two separate antibiotic-free selection methods in Syn7002. A strain produced by double selection may be more robust against contaminating organisms. Therefore, Mel5, one of the best melamine-growing strains, was transformed with either pSJ135 (ptxD gene alone; SEQ ID NO: 93) or pSJ141 (ptxD and yfp) and selected for positive transformants on AD7-Mel 1×Phi plates. As was the case for the Syn7002 WT background, positive transformants could be readily obtained by this selection method (FIG. 7B) and YFP fluorescence measured in the fully segregated YFP-expressing transformants (FIG. 7C). Strain Mel5-A0935ptxD (“MelPhi”) was able to grow in AD7-Mel Phi 20× liquid medium, albeit at a slightly slower rate, in comparison to MelPhi or the Syn7002 WT parental strain grown in regular AD7 (FIG. 9A). As predicted, Syn7002 WT was unable to grow in AD7-Mel Phi 20× medium (FIG. 9A). This shows that a double selection using both melamine and Phi is possible in the same chassis. Growth of Syn7002, Mel5, A0935ptxD and MelPhi strains on plates comprising various media is shown in FIG. 13.

### Example 5

### Strain Mel5-A0935ptxD is Able to Resist Deliberate Contamination

The present invention relates to a cyanobacterial strain that would be suitable for outdoor cultivation in open systems. This strain should be able to outcompete other strains under these conditions to become the dominant population in a potentially contaminated system. To determine the robustness of the obtained strain, an experiment was devised where the starting culture for Mel5-A0935ptxD (in AD7-Mel Phi 20×) was deliberately contaminated with a large excess, either 6 times (FIG. 9C) or 10 times higher cell counts (FIG. 11) of a Syn7002 strain constitutively expressing YFP (from the P_{cpt} promoter). As can be seen in FIG. 9C, Mel5-A0935ptxD (“MelPhi”) was able to overcome this large excess of contaminant and become the dominant population, thus showing that this strain is indeed suitable for outdoor cultivation under unsterilized conditions. Though, as discussed above, there is a leakage of melamine pathway intermediates from the parental strain Mel5 cells during cultivation in melamine (FIG. 4), the low amounts of these xenobiotic compounds released by the cells (at maximum 0.2 mM) are very unlikely to support bacterial growth, thus negating any form of scavenging from contaminant cells. The slight growth of the YFP contaminant observed is, as mentioned above (see Example 3), most likely due to mobilization and consumption of internal nutrient reserves, though this is unable to sustain long term growth. Previously, transgenic A. thaliana expressing ptxD was also shown to be able to resist and overcome deliberate contamination by common weeds [Lopez-Arredondo and Herrera-Estrella, Nat Biotechnol. 30: 889 (2012)], underscoring the advantage of this approach to give strains grown in unsterilized conditions an edge over the competition.

### Example 6

### Growth Scalability of Strain Mel5-A0935ptxD

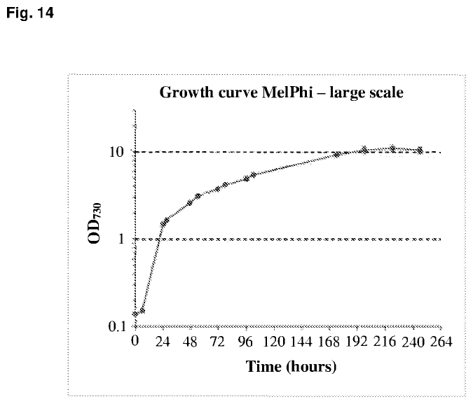

The ability of Me/5-A0935ptxD to grow in larger scale cultures was tested by growing in 2×1 L baffled Erlenmeyer flasks (total volume of 2 L) in AD7-Mel Phi 20× media in a growth chamber for 11 days. The bacteria grew quite fast in the first 24 hours and tapered off to an OD of about 10 (FIG. 14). An explanation of the slowing growth rate is the dilute cultures allow more light in, in the first 24 hours, and become essentially light limited beyond that stage as the cultures become more turbid, hence the slower growth.

### Example 7

### Construction of a MelPhi Strain Capable of Utilizing NADP+ Instead of NAD+

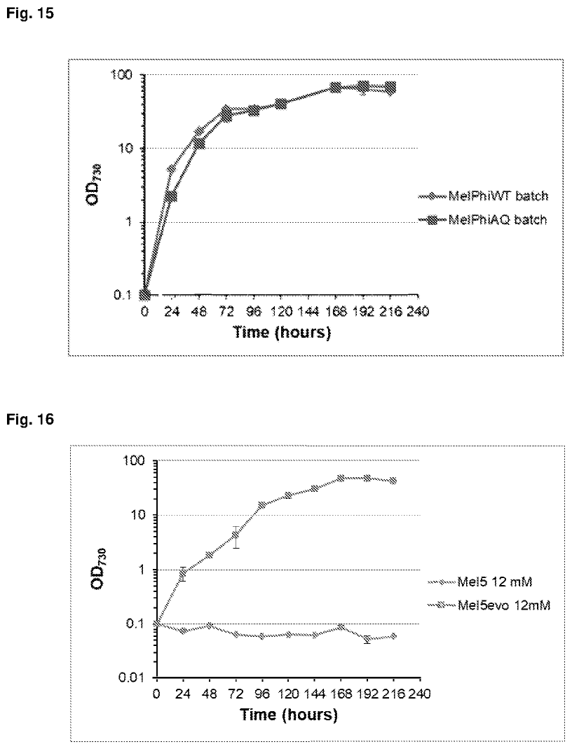

As cyanobacteria have more NADP+ than NAD+a new derivative of the MelPhi strain was generated in which the PtxD enzyme (codon optimized; SEQ ID NO: 90) was mutated to use NADP+ instead of NAD+ with the polynucleotide sequence of the mutated gene set forth in SEQ ID NO: 91. The sequence of the ptxD gene was mutated using primers RF_ptxD_F (SEQ ID NO: 95) and RF_ptxD_R (SEQ ID NO: 96), using pSJ135 as template, Phusion polymerase (NEB) and the RF-cloning method as described in van den Ent, et al., J Biochem Biophys Methods, 67: 67-74 (2006), resulting in plasmid pSJ165. The mutations in the ptxD gene (Glu175Ala and Ala176Arg) emulated those described in Woodyer, R. et al., FEBS J., 272: 3816-27 (2005), previously shown to increase specificity of PtxD to NADP+. Mel5 was transformed with pSJ165 as described above. The ptxD mutant strain was designated MelPhiAQ and its growth was compared to MelPhi using a fed-batch strategy, adding melamine every day to continue growing to higher densities. FIG. 15 shows both strains grew similarly and the highest density reached was around an OD₇₃₀ of 70.

### Example 8

Evolution of a Mel5 Strain that can Grow in High Concentration of Melamine

The Mel5 strain was evolved in a media comprising 2 mM melamine and could grow in 4 mM melamine (FIG. 5). A strain that could grow in higher concentrations of melamine may be even more resistant to contamination, so the Mel5 strain was further evolved on 12 mM melamine. A Mel5 culture was plated on AD7-Mel plates containing 12 mM melamine instead of the regular 2 mM and cultured as described above. After approximately 2 weeks colonies appeared that were restreaked in AD7-Mel plates with 12 mM melamine a further 4 times under the same conditions. Of the initial 12 colonies streaked, the 3 seemingly more robust growing strains (on plate) were cultured in AD7 liquid medium with 12 mM melamine as nitrogen source. The growth of the most robust strain, the newly evolved Mel5 strain (“Mel5evo”) thus generated, was compared to Mel5. As shown in FIG. 16, the original strain cannot grow in 12 mM melamine but the evolved strain grows to fairly high OD₇₅₀ (OD around 50). It appears the improved growth in high melamine concentrations was due to further mutations in the triA gene. The polynucleotide sequence of the triA gene is set forth in SEQ ID NO: 69, and the amino acid sequence is set forth in SEQ ID NO: 68. There are two further amino acid substitutions, at Thr218Asn and Val278Met, in comparison to the triA sequence in Mel5.

### SUMMARY

An important consideration of the engineered strains of the invention is that, at current prices and at the concentrations utilized in this study, melamine would be a more economical nitrogen source than nitrate, with a 24% reduction in cost when using melamine (see Table 3).

TABLE 3
Cost estimate of nitrate, melamine, phosphate and phosphite

| | Lowest | | |
|---|---|---|---|
| | bulk | Amount (kg) | Price (USD) |
| | price | per 1000 L | per 1000 L |
| | (USD/kg) | of medium | of medium |
| Sodium nitrate | 0.1 | 1 | 0.1 |
| Melamine | 0.3 | 0.252 | 0.076 |
| Monopotassium | 0.1 | 0.05 | 0.005 |
| Phosphate (1×) | | | |
| Monopotassium | 0.1 | 0.889 | 0.089 |
| Phosphite (20×) | | | |

Note:
Values are minimum prices, based on data from the Alibaba.com website (accessed 25th March 2019)

Also, as melamine is not used as an agricultural fertilizer, its usage as nitrogen source would eliminate competition for nitrogen-rich fertilizers used in agriculture. Furthermore, as melamine levels drop to below the level of detection using LC-MS/MS within 24 hours of growth, residual melamine in the final culture supernatant would not be a deterring factor in adoption of this technique.

The additional selection by phosphite gives the strain a “double edge” that will be even more difficult to overcome by contaminating species, especially in the early stages of the cultures, thus allowing strains carrying these two modifications to become the dominant population without the need for sterilization or antibiotic addition. Furthermore, this strategy negates the risk for horizontal gene transfer of antibiotic resistance cassettes [Ventola, P T. 40: 277-83 (2015a); Ventola, P T. 40: 344-52 (2015b); von Wintersdorff et al., Frontiers in microbiology. 7: 10.3389/fmicb.2016.00173 (2016)].

The results herein indicate that TriA mutations regulate flux through the melamine pathway so that it becomes more efficiently used. The Mel5evo strain allows batch culture to high density without the need for online feeding equipment, etc (more expensive than batch cultures). The MelPhiAQ strain grows well and may improve production of biomolecules needing higher NADPH concentrations (as phosphite conversion to phosphate using the mutated PtxD enzyme will convert NADP+ to NADPH, thereby increasing its internal concentration).

The strains of the present invention provide two different strategies for high-density cultivation. The first strategy is fed-batch using Mel5 and related engineered cyanobacterium strains. The second strategy is batch culture with a high concentration of melamine (up to at least 12 mM) using the Mel5evo cyanobacterium strain.

In conclusion, this work describes, for the first time, marine cyanobacterial strains that are able to grow on up to 12 mM melamine as sole nitrogen source, the use of phosphite selection as an efficient selection strategy in cyanobacteria and a phosphite metabolizing strain that can utilize NADP+ instead of NAD+. Finally, we developed a unique strain that is able to use both melamine and phosphite as sole N and P sources, respectively. This strain is able to resist deliberate contamination by other cyanobacteria, even when the contamination is present in large excess, and should prove to be a useful chassis strain for “green” biotechnological applications.

### REFERENCES

Angermayr, S. A., Paszota, M., Hellingwerf, K. J., 2012. Engineering a cyanobacterial cell factory for production of lactic acid. Applied and environmental microbiology. 78, 7098-106.10.1128/A EM.01587-12.
Arnon, D. I., McSwain, B. D., Tsujimoto, H. Y., Wada, K., 1974. Photochemical activity and components of membrane preparations from blue-green algae. I. Coexistence of two photosystems in relation to chlorophyll a and removal of phycocyanin. Biochim Biophys Acta. 357, 231-45.
Begemann, M. B., Zess, E. K., Walters, E. M., Schmitt, E. F., Markley, A. L., Pfleger, B. F., 2013. An organic acid based counter selection system for cyanobacteria. PLoS One. 8, e76594.10.1371/journal.pone.0076594.
Bisson, C., Adams, N. B. P., Stevenson, B., Brindley, A. A., Polyviou, D., Bibby, T. S., Baker, P. J., Hunter, C. N., Hitchcock, A., 2017. The molecular basis of phosphite and hypophosphite recognition by ABC-transporters. Nat Commun. 8, 1746.10.1038/s41467-017-01226-8.
Braekevelt, E., Lau, B. P., Feng, S., Menard, C., Tittlemier, S. A., 2011. Determination of melamine, ammeline, ammelide and cyanuric acid in infant formula purchased in Canada by liquid chromatography-tandem mass spectrometry. Food Addit Contam Part A Chem Anal Control Expo Risk Assess. 28, 698-704.10.1080/19440049.2010.545442.
Choi, S. Y., Wang, J. Y., Kwak, H. S., Lee, S. M., Um, Y., Kim, Y., Sim, S. J., Choi, J. I., Woo, H. M., 2017. Improvement of squalene production from CO₂ in Synechococcus elongatus PCC 7942 by metabolic engineering and scalable production in a photobioreactor. ACS Synth Biol. 6, 1289-1295.10.1021/acssynbio.7b00083.
Clark, R. L., McGinley, L. L., Purdy, H. M., Korosh, T. C., Reed, J. L., Root, T. W., Pfleger, B. F., 2018. Light-optimized growth of cyanobacterial cultures: Growth phases and productivity of biomass and secreted molecules in light-limited batch growth. Metab Eng. 47, 230-242.10.1016/j.ymben.2018.03.017.
Collier, J. L., Grossman, A. R., 1992. Chlorosis induced by nutrient deprivation in Synechococcus sp. strain PCC 7942: not all bleaching is the same. Journal of bacteriology. 174, 4718-26.
Davies, F. K., Work, V. H., Beliaev, A. S., Posewitz, M. C., 2014. Engineering limonene and bisabolene production in wild type and a glycogen-deficient mutant of Synechococcus sp. PCC 7002. Frontiers in bioengineering and biotechnology. 2, 21.10.3389/fbioe.2014.00021.
Dexter, J., Armshaw, P., Sheahan, C., Pembroke, J. T., 2015. The state of autotrophic ethanol production in Cyanobacteria. J Appl Microbiol. 119, 11-24.10.1111/jam.12821.
Elhai, J., Wolk, C. P., 1988. A versatile class of positive-selection vectors based on the nonviability of palindrome-containing plasmids that allows cloning into long polylinkers. Gene. 68, 119-138.Doi 10.1016/0378-1119(88)90605-1.
Englund, E., Pattanaik, B., Ubhayasekera, S. J., Stensjo, K., Bergquist, J., Lindberg, P., 2014. Production of squalene in Synechocystis sp. PCC 6803. PLoS One. 9, e90270.10.1371/journal.pone.0090270.
Fathima, A. M., Chuang, D., Lavina, W. A., Liao, J., Putri, S. P., Fukusaki, E., 2018. Iterative cycle of widely targeted metabolic profiling for the improvement of 1-butanol titer and productivity in Synechococcus elongatus. Biotechnol Biofuels. 11, 188.10.1186/s13068-018-1187-8.
Feingersch, R., Philosof, A., Mejuch, T., Glaser, F., Alalouf, O., Shoham, Y., Beja, O., 2012. Potential for phosphite and phosphonate utilization by Prochlorococcus. ISME J. 6, 827-34.10.1038/ismej.2011.149.
Frigaard, N. U., Sakuragi, Y., Bryant, D. A., 2004. Gene inactivation in the cyanobacterium Synechococcus sp. PCC 7002 and the green sulfur bacterium Chlorobium tepidum using in vitro-made DNA constructs and natural transformation. Methods Mol Biol. 274, 325-40.10.1385/1-59259-799-8:325.
Gomez-Garcia, M. R., Fazeli, F., Grote, A., Grossman, A. R., Bhaya, D., 2013. Role of polyphosphate in thermophilic Synechococcus sp. from microbial mats. Journal of bacteriology. 195, 3309-19.10.1128/JB.00207-13.
Gordon, G. C., Korosh, T. C., Cameron, J. C., Markley, A. L., Begemann, M. B., Pfleger, B. F., 2016. CRISPR interference as a titratable, trans-acting regulatory tool for metabolic engineering in the cyanobacterium Synechococcus sp. strain PCC 7002. Metab Eng. 38, 170-179.10.1016/j.ymben.2016.07.007.
Halfmann, C., Gu, L., Gibbons, W., Zhou, R., 2014. Genetically engineering cyanobacteria to convert CO₂, water, and light into the long-chain hydrocarbon farnesene. Appl Microbiol Biotechnol. 98, 9869-77.10.1007/s00253-014-6118-4.
Hirota, R., Abe, K., Katsuura, Z. I., Noguchi, R., Moribe, S., Motomura, K., Ishida, T., Alexandrov, M., Funabashi, H., Ikeda, T., Kuroda, A., 2017. A novel biocontainment strategy makes bacterial growth and survival dependent on phosphite. Sci Rep. 7, 44748.10.1038/srep44748.
Kanda, K., Ishida, T., Hirota, R., Ono, S., Motomura, K., Ikeda, T., Kitamura, K., Kuroda, A., 2014. Application of a phosphite dehydrogenase gene as a novel dominant selection marker for yeasts. J Biotechnol. 182, 68-73.10.1016/j.jbiotec.2014.04.012.
Kato, A., Takatani, N., Ikeda, K., Maeda, S. I., Omata, T., 2017. Removal of the product from the culture medium strongly enhances free fatty acid production by genetically engineered Synechococcus elongatus. Biotechnol Biofuels. 10, 141.10.1186/s13068-017-0831-z.
Liu, H., Naismith, J. H., 2008. An efficient one-step site-directed deletion, insertion, single and multiple-site plasmid mutagenesis protocol. BMC Biotechnol. 8, 91.10.1186/1472-6750-8-91.
Loera-Quezada, M. M., Leyva-Gonzalez, M. A., Velazquez-Juarez, G., Sanchez-Calderon, L., Do Nascimento, M., Lopez-Arredondo, D., Herrera-Estrella, L., 2016. A novel genetic engineering platform for the effective management of biological contaminants for the production of microalgae. Plant Biotechnol J. 14, 2066-76.10.1111/pbi.12564.
Lopez-Arredondo, D. L., Herrera-Estrella, L., 2012. Engineering phosphorus metabolism in plants to produce a dual fertilization and weed control system. Nat Biotechnol. 30, 889-93.10.1038/nbt.2346.
Ludwig, M., Bryant, D. A., 2011. Transcription profiling of the model cyanobacterium Synechococcus sp. strain PCC 7002 by next-gen (SOLiD) sequencing of cDNA. Frontiers in microbiology. 2, 41.
Ludwig, M., Bryant, D. A., 2012. Synechococcus sp. strain PCC 7002 transcriptome: acclimation to temperature, salinity, oxidative stress, and mixotrophic growth conditions. Frontiers in microbiology. 3, 354.
Markley, A. L., Begemann, M. B., Clarke, R. E., Gordon, G. C., Pfleger, B. F., 2015. Synthetic biology toolbox for controlling gene expression in the cyanobacterium Synechococcus sp. strain PCC 7002. ACS Synth Biol. 4, 595-603.10.1021/sb500260k.
Martinez, A., Osburne, M. S., Sharma, A. K., DeLong, E. F., Chisholm, S. W., 2012. Phosphite utilization by the marine picocyanobacterium Prochlorococcus MIT9301. Environ Microbiol. 14, 1363-77.10.1111/j.1462-2920.2011.02612.x.
Motomura, K., Sano, K., Watanabe, S., Kanbara, A., Gamal Nasser, A. H., Ikeda, T., Ishida, T., Funabashi, H., Kuroda, A., Hirota, R., 2018. Synthetic phosphorus metabolic pathway for biosafety and contamination management of cyanobacterial cultivation. ACS Synth Biol.10.1021/acssynbio.8b00199.
Nahampun, H. N., Lopez-Arredondo, D., Xu, X., Herrera-Estrella, L., Wang, K., 2016. Assessment of ptxD gene as an alternative selectable marker for Agrobacterium-mediated maize transformation. Plant Cell Rep. 35, 1121-1132.10.1007/s00299-016-1942-x.
Pandeya, D., Campbell, L. M., Nunes, E., Lopez-Arredondo, D. L., Janga, M. R., Herrera-Estrella, L., Rathore, K. S., 2017. ptxD gene in combination with phosphite serves as a highly effective selection system to generate transgenic cotton (Gossypium hirsutum L.). Plant Mol Biol. 95, 567-577.10.1007/s11103-017-0670-0.
Perez, A. A., Liu, Z., Rodionov, D. A., Li, Z., Bryant, D. A., 2016. Complementation of cobalamin auxotrophy in Synechococcus sp. strain PCC 7002 and validation of a putative cobalamin riboswitch in vivo. Journal of bacteriology.10.1128/JB.00475-16.
Polyviou, D., Hitchcock, A., Baylay, A. J., Moore, C. M., Bibby, T. S., 2015. Phosphite utilization by the globally important marine diazotroph Trichodesmium. Environmental microbiology reports. 7, 824-30.10.1111/1758-2229.12308.
Ruffing, A. M., 2014. Improved free fatty acid production in cyanobacteria with Synechococcus sp. PCC 7002 as host. Frontiers in bioengineering and biotechnology. 2, 17.10.3389/fbioe.2014.00017.
Schoepp, N. G., Stewart, R. L., Sun, V., Quigley, A. J., Mendola, D., Mayfield, S. P., Burkart, M. D., 2014. System and method for research-scale outdoor production of microalgae and cyanobacteria. Bioresour Technol. 166, 273-81.10.1016/j.biortech.2014.05.046.
Shabestary, K., Anfelt, J., Ljungqvist, E., Jahn, M., Yao, L., Hudson, E. P., 2018. Targeted repression of essential genes to arrest growth and increase carbon partitioning and biofuel titers in cyanobacteria. ACS Synth Biol. 7, 1669-1675.10.1021/acssynbio.8b00056.
Shaw, A. J., Lam, F. H., Hamilton, M., Consiglio, A., MacEwen, K., Brevnova, E. E., Greenhagen, E., LaTouf, W. G., South, C. R., van Dijken, H., Stephanopoulos, G., 2016. Metabolic engineering of microbial competitive advantage for industrial fermentation processes. Science. 353, 583-6.10.1126/science.aaf6159.
Stevens, S. E., Patterson, C. O., Myers, J., 1973. Production of hydrogen peroxide by blue-green algae—a survey. J Phycol. 9, 427-430
van den Ent, F., and Löwe J., 2006. RF cloning: A restriction-free method for inserting target genes into plasmids. J Biochem Biophys Methods. April 30; 67(1): 67-74.
Ventola, C. L., 2015a. The antibiotic resistance crisis: part 1: causes and threats. P T. 40, 277-83.
Ventola, C. L., 2015b. The antibiotic resistance crisis: part 2: management strategies and new agents. P T. 40, 344-52.
von Wintersdorff, C. J. H., Penders, J., van Niekerk, J. M., Mills, N. D., Majumder, S., van Alphen, L. B., Savelkoul, P. H. M., Wolffs, P. F. G., 2016. Dissemination of antimicrobial resistance in microbial ecosystems through horizontal gene transfer. Frontiers in microbiology. 7.10.3389/fmicb.2016.00173.
Wang, X., Liu, W., Xin, C., Zheng, Y., Cheng, Y., Sun, S., Li, R., Zhu, X. G., Dai, S. Y., Rentzepis, P. M., Yuan, J. S., 2016. Enhanced limonene production in cyanobacteria reveals photosynthesis limitations. Proceedings of the National Academy of Sciences of the United States of America. 113, 14225-14230.10.1073/pnas.1613340113.
Woodyer R., Zhao H., van der Donk W A., 2005. Mechanistic investigation of a highly active phosphite dehydrogenase mutant and its application for NADPH regeneration. FEBS J. August; 272(15): 3816-27.
Xu, Y., Alvey, R. M., Byrne, P. O., Graham, J. E., Shen, G., Bryant, D. A., 2011. Expression of genes in cyanobacteria: adaptation of endogenous plasmids as platforms for high-level gene expression in Synechococcus sp. PCC 7002. Methods Mol Biol. 684, 273-93.

1. An isolated genetically engineered cyanobacterium, wherein the cyanobacterium has been transformed by at least one polynucleotide molecule; the at least one polynucleotide molecule comprising heterologous melamine utilization pathway genes, atzD, trzE, DUR1,2, trzC, guaD and triA operably linked to at least one promoter, wherein;
i) the triA gene comprises one or more mutations which encode amino acid substitutions, wherein the amino acid substitutions are at positions selected from the group comprising Leu88Phe, His254Tyr, Glu317Lys, Ala355Val, Trp471Stop and the combination of Thr218Asn and Val278Met;
and/or
ii) the triA gene has a ribosome binding site (RBS) comprising a AGGAGA to AGAAGA mutation,
wherein said genetically engineered cyanobacterium has no heterologous antibiotic resistance genes.

2. The isolated genetically engineered cyanobacterium of claim 1, wherein the triA gene encodes an amino acid sequence selected from the group comprising SEQ ID NO: 56, SEQ ID NO: 58, SEQ ID NO: 60, SEQ ID NO: 62, SEQ ID NO: 64, SEQ ID NO: 66 and SEQ ID NO: 68; and/or
wherein the triA gene comprises a polynucleotide sequence which has at least 80%, at least 85%, at least 90%, at least 95% sequence identity or 100% sequence identity to the polynucleotide sequence selected from the group comprising SEQ ID NO: 57, SEQ ID NO: 59, SEQ ID NO: 61, SEQ ID NO: 63, SEQ ID NO: 65, SEQ ID NO: 67, SEQ ID NO: 69 and SEQ ID NO: 70.

3. (canceled)

4. The isolated genetically engineered cyanobacterium of claim 1, wherein the heterologous gene trzE comprises a polynucleotide sequence which has at least 80%, at least 85%, at least 90%, at least 95% sequence identity or 100% sequence identity to the polynucleotide sequence set forth in SEQ ID NO: 71 or SEQ ID NO: 72;
trzC comprises a polynucleotide sequence which has at least 80%, at least 85%, at least 90%, at least 95% sequence identity or 100% sequence identity to the polynucleotide sequence set forth in SEQ ID NO: 73 or SEQ ID NO: 74;
DUR1,2 comprises a polynucleotide sequence which has at least 80%, at least 85%, at least 90%, at least 95% sequence identity or 100% sequence identity to the polynucleotide sequence set forth in SEQ ID NO: 75 or SEQ ID NO: 76;
atzD comprises a polynucleotide sequence which has at least 80%, at least 85%, at least 90%, at least 95% sequence identity or 100% sequence identity to the polynucleotide sequence set forth in SEQ ID NO: 77 or SEQ ID NO: 78;
guaD comprises a polynucleotide sequence which has at least 80%, at least 85%, at least 90%, at least 95% sequence identity or 100% sequence identity to the polynucleotide sequence set forth in SEQ ID NO: 79, SEQ ID NO: 80 or SEQ ID NO: 81.

5. The isolated genetically engineered cyanobacterium of claim 1, wherein each of said melamine utilization pathway genes has a ribosome binding site (RBS) for each of said melamine utilization pathway genes; and/or wherein said at least one promoter is a constitutive promoter.

6. (canceled)

7. The isolated genetically engineered cyanobacterium of claim 1, wherein said heterologous melamine utilization pathway genes are expressed from a single promoter as a part of a gene operon.

8. The isolated genetically engineered cyanobacterium of claim 7, wherein the gene operon polynucleotide sequence is selected from the group comprising SEQ ID NO: 83, SEQ ID NO: 84, SEQ ID NO: 85, SEQ ID NO: 86, SEQ ID NO: 87 and SEQ ID NO: 88.

9. The isolated genetically engineered cyanobacterium of claim 1, wherein the at least one polynucleotide molecule further comprises a polynucleotide comprising a heterologous phosphite dehydrogenase (ptxD) gene operably linked to a promoter.

10. The isolated genetically engineered cyanobacterium of claim 9, wherein the ptxD gene comprises a polynucleotide sequence set forth in SEQ ID NO: 89, SEQ ID NO: 90, or SEQ ID NO: 91; and/or
wherein said heterologous phosphite dehydrogenase (ptxD) gene is expressed from a single promoter as a part of a gene operon, wherein the operon polynucleotide sequence is set forth in SEQ ID NO: 93.

11. (canceled)

12. An isolated genetically engineered cyanobacterium, wherein the cyanobacterium has been transformed by at least one polynucleotide molecule; the at least one polynucleotide molecule comprising a heterologous phosphite dehydrogenase (ptxD) gene operably linked to a promoter, wherein the ptxD gene comprises a polynucleotide sequence set forth in SEQ ID NO: 90, or SEQ ID NO: 91,
wherein said genetically engineered cyanobacterium has no heterologous antibiotic resistance genes.

13. The isolated genetically engineered cyanobacterium of claim 1, further comprising an exogenous polynucleotide comprising an expressible polynucleotide encoding an RNA and/or a protein product.

14. The isolated genetically engineered cyanobacterium of claim 13, wherein the cyanobacterium is a Synechococcus sp.

15. A recombinant vector comprising melamine pathway genes triA, DUR1,2, atzD, trzC, trzE, and guaD, operably linked to at least one promoter, wherein
i) the triA gene comprises one or more mutations which encode amino acid substitutions, wherein the amino acid substitutions are at positions selected from the group comprising Leu88Phe, His254Tyr, Glu317Lys, Ala355Val, Trp471Stop, and the combination of Thr218Asn and Val278Met;
and/or
ii) the triA gene has a ribosome binding site (RBS) comprising a AGGAGA to AGAAGA mutation,
wherein the vector lacks antibiotic resistance genes.

16. The recombinant vector of claim 15, wherein the triA gene encodes an amino acid sequence selected from the group comprising SEQ ID NO: 56, SEQ ID NO: 58, SEQ ID NO: 60, SEQ ID NO: 62, SEQ ID NO: 64, SEQ ID NO: 66 and SEQ ID NO: 68; and/or
wherein the triA gene comprises a polynucleotide sequence which has at least 80%, at least 85%, at least 90%, at least 95% sequence identity or 100% sequence identity to the polynucleotide sequence of the triA gene selected from the group comprising SEQ ID NO: 57, SEQ ID NO: 59, SEQ ID NO: 61, SEQ ID NO: 63, SEQ ID NO: 65, SEQ ID NO: 67, SEQ ID NO: 69 and SEQ ID NO: 70.

17. (canceled)

18. The recombinant vector of claim 15, wherein the heterologous gene trzE comprises a polynucleotide sequence which has at least 80%, at least 85%, at least 90%, at least 95% sequence identity or 100% sequence identity to the polynucleotide sequence set forth in SEQ ID NO: 71 or SEQ ID NO: 72;
trzC comprises a polynucleotide sequence which has at least 80%, at least 85%, at least 90%, at least 95% sequence identity or 100% sequence identity to the polynucleotide sequence set forth in SEQ ID NO: 73 or SEQ ID NO: 74;
DUR1,2 comprises a polynucleotide sequence which has at least 80%, at least 85%, at least 90%, at least 95% sequence identity or 100% sequence identity to the polynucleotide sequence set forth in SEQ ID NO: 75 or SEQ ID NO: 76;
atzD comprises a polynucleotide sequence which has at least 80%, at least 85%, at least 90%, at least 95% sequence identity or 100% sequence identity to the polynucleotide sequence set forth in SEQ ID NO: 77 or SEQ ID NO: 78;
guaD comprises a polynucleotide sequence which has at least 80%, at least 85%, at least 90%, at least 95% sequence identity or 100% sequence identity to the polynucleotide sequence set forth in SEQ ID NO: 79, SEQ ID NO: 80 or SEQ ID NO: 81.

19. The recombinant vector of claim 15, wherein each of said melamine utilization pathway genes has a ribosome binding site (RBS); and/or
wherein said at least one promoter is a constitutive promoter.

20. (canceled)

21. The recombinant vector of claim 15, wherein said heterologous melamine utilization pathway genes are expressed from a single promoter as a part of a gene operon.

22. The recombinant vector of claim 21, wherein the gene operon polynucleotide sequence is selected from the group comprising SEQ ID NO: 83, SEQ ID NO: 84, SEQ ID NO: 85, SEQ ID NO: 86, SEQ ID NO: 87 and SEQ ID NO: 88.

23. The recombinant vector of claim 15, wherein the at least one polynucleotide molecule further comprises a polynucleotide comprising a heterologous phosphite dehydrogenase (ptxD) gene operably linked to a promoter; and/or
wherein the ptxD gene comprises a polynucleotide sequence set forth in SEQ ID NO: 89, SEQ ID NO: 90 or SEQ ID NO: 91.

24. (canceled)

25. The recombinant vector of claim 15, further comprising an exogenous polynucleotide comprising an expressible polynucleotide encoding an RNA and/or a protein product.

26. A method of expressing a product in a genetically engineered cyanobacterium cell, comprising the steps:
a) culturing a plurality of genetically engineered cyanobacteria cells of claim 1 in medium where there is no antibiotic and melamine is the nitrogen source, wherein culturing favours growth of cyanobacterium cells that metabolise melamine; and wherein said engineered cyanobacteria cells further comprise at least one exogenous polynucleotide comprising an expressible polynucleotide encoding an RNA and/or a protein product, and
b) culturing said genetically engineered cyanobacterium cells under conditions for expression of said product; or
c) culturing a plurality of genetically engineered cyanobacterium cells of claim 1 wherein the at least one polynucleotide molecule further comprises a polynucleotide comprising a heterologous phosphite dehydrogenase (PtxD) gene operably linked to a promoter in medium where there is no antibiotic, melamine is the nitrogen source and phosphite is the phosphorous source, wherein culturing favours growth of cyanobacterium cells that metabolise melamine and phosphite; and wherein said engineered cyanobacteria cells further comprise at least one exogenous polynucleotide comprising an expressible polynucleotide encoding an RNA and/or a protein product, and
d) culturing said genetically engineered cyanobacterium cells under conditions for expression of said product.

27. (canceled)

28. (canceled)

29. The method of claim 26, further comprising isolating said product expressed in the genetically engineered cyanobacterium cell.


SEQUENCE LISTING (96 sequences)

SEQ ID NO: 1 - DNA, 20 bp, Artificial Sequence
  D08807
gcactgtggcaaggaaatcg

SEQ ID NO: 2 - DNA, 20 bp, Artificial Sequence
  D08808
tcgcctttatggaggatggc

SEQ ID NO: 3 - DNA, 28 bp, Artificial Sequence
  D98496993
taaaaaagactttatgactgctttactg

SEQ ID NO: 4 - DNA, 22 bp, Artificial Sequence
  D77036
ggctcaaaagacatcatttagg

SEQ ID NO: 5 - DNA, 70 bp, Artificial Sequence
  D98847023
gaaggttttctgttatctgaaattcctccctaaatgatgtcttttgagccaaaggtgcttgtgtctcaac

SEQ ID NO: 6 - DNA, 46 bp, Artificial Sequence
  D98847024
tcatggtgtatatctcctaatcaatttagagcatttcaaagtaggc

SEQ ID NO: 7 - DNA, 49 bp, Artificial Sequence
  D98847025
tcgggcctactttgaaatgctctaaattgattaggagatatacaccatg

SEQ ID NO: 8 - DNA, 69 bp, Artificial Sequence
  D98847026
gggaataatgttggtcatggagcagtaaagcagtcataaagtcttttttatcgctcgtcatttgctttc

SEQ ID NO: 9 - DNA, 29 bp, Artificial Sequence
  D99280067
tgaagatcaacaccatctatgacttagcc

SEQ ID NO: 10 - DNA, 21 bp, Artificial Sequence
  D99280068
taaccccatgcaaacgccatc

SEQ ID NO: 11 - DNA, 24 bp, Artificial Sequence
  Mel_seq_1
tgccattggtatgagtgaacaagc

SEQ ID NO: 12 - DNA, 21 bp, Artificial Sequence
  Mel_seq_2
tctctccgattgtaccggtgc

SEQ ID NO: 13 - DNA, 34 bp, Artificial Sequence
  Mel_seq_3
aatggatcaaatcttttatttgtgaagaaagtgg

SEQ ID NO: 14 - DNA, 17 bp, Artificial Sequence
  Mel_seq_4
atgatgtggcccgctcc

SEQ ID NO: 15 - DNA, 29 bp, Artificial Sequence
  Mel_seq_5
tcgtggataaactctttgtgatgatgacc

SEQ ID NO: 16 - DNA, 21 bp, Artificial Sequence
  Mel_seq_6
catgcccaaattaccgaaacc

SEQ ID NO: 17 - DNA, 29 bp, Artificial Sequence
  Mel_seq_7
tgctctctgatagtgatattaattccacc

SEQ ID NO: 18 - DNA, 26 bp, Artificial Sequence
  Mel_seq_8
gaaggtgaacccattattgataaacc

SEQ ID NO: 19 - DNA, 28 bp, Artificial Sequence
  Mel_seq_9
tccatcaatttcaaattctcaagtctcg

SEQ ID NO: 20 - DNA, 27 bp, Artificial Sequence
  Mel_seq_10
agcaatcccctcaaaaaatttagttcc

SEQ ID NO: 21 - DNA, 28 bp, Artificial Sequence
  Mel_seq_11
tcattggcaaaaaatttaccgattatgc

SEQ ID NO: 22 - DNA, 18 bp, Artificial Sequence
  Mel_seq_12
tgcccaatgtgaccgtgg

SEQ ID NO: 23 - DNA, 25 bp, Artificial Sequence
  Mel_seq_13
tcaaagaagaataccccgatacctg

SEQ ID NO: 24 - DNA, 21 bp, Artificial Sequence
  Mel_seq_14
tcgatgaacgcctccaagtgg

SEQ ID NO: 25 - DNA, 36 bp, Artificial Sequence
  D100023580
acccggggatcctctagagccagatccttttgcatc

SEQ ID NO: 26 - DNA, 39 bp, Artificial Sequence
  D100023581
ctgcaggtcgactcttgaagggagccagaacataaaaag

SEQ ID NO: 27 - DNA, 33 bp, Artificial Sequence
  A0935_UCO_F
tgacttggttcacgtagaaaaaccagaagggac

SEQ ID NO: 28 - DNA, 51 bp, Artificial Sequence
  A0936_UCO_R
tgtccactccttaatcactattcaaaatattatatttactcagtttttaag

SEQ ID NO: 29 - DNA, 21 bp, Artificial Sequence
  D98496996
attaaggagtggacagcctac

SEQ ID NO: 30 - DNA, 22 bp, Artificial Sequence
  D100141467
acgtgaaccaagtcagacaatc

SEQ ID NO: 31 - DNA, 32 bp, Artificial Sequence
  D100043610
tagatgatgatgtagaattgtctgctaattac

SEQ ID NO: 32 - DNA, 27 bp, Artificial Sequence
  D100043611
taatgatttcagtacaaattgctctgc

SEQ ID NO: 33 - DNA, 52 bp, Artificial Sequence
  D100263687
tttgattgtctgacttggttcacgtttgaggccgttgatctagacaaaaaac

SEQ ID NO: 34 - DNA, 24 bp, Artificial Sequence
  D100263688
aggagattaattccatgggccatc

SEQ ID NO: 35 - DNA, 19 bp, Artificial Sequence
  D100098818
agaaaaaccagaagggacg

SEQ ID NO: 36 - DNA, 53 bp, Artificial Sequence
  D101108991
taaagtcaagtagaagattaattccatgggccatcatcatcatcatcatcatc

SEQ ID NO: 37 - DNA, 50 bp, Artificial Sequence
  D101108992
ggaattaatcttctacttgactttatgagttgggagctccttttgcaatc

SEQ ID NO: 38 - DNA, 24 bp, Artificial Sequence
  D74727
ttcacagaggaaggggaaattgtc

SEQ ID NO: 39 - DNA, 22 bp, Artificial Sequence
  D74729
tcatatccggggcatacattcg

SEQ ID NO: 40 - DNA, 50 bp, Artificial Sequence
  D101108989
ttaagctagttagaagattcagaccatgcaaaccctcagcattcaacatg

SEQ ID NO: 41 - DNA, 47 bp, Artificial Sequence
  D101108990
ggtctgaatcttctaactagcttaattccgttcgtacacgaggcgac

SEQ ID NO: 42 - DNA, 46 bp, Artificial Sequence
  D15106
gttgtaaaacgacggccagtgaattttaagcctggggtgagttgac

SEQ ID NO: 43 - DNA, 58 bp, Artificial Sequence
  D15107
cgtttccagttgtccactccttaattaatttctcaagggaaagaaaaagatttattcc

SEQ ID NO: 44 - DNA, 52 bp, Artificial Sequence
  D15108
ttaaaaagcatgacttggttcacgtacgaaggttgtttttaaagctaagaag

SEQ ID NO: 45 - DNA, 52 bp, Artificial Sequence
  D15109
caggaaacagctatgaccatgattatatgaaaatgcttcacaccatgattcg

SEQ ID NO: 46 - DNA, 52 bp, Artificial Sequence
  D15110
gttgtaaaacgacggccagtgaattacaattgacacaaaaaagagagcaaag

SEQ ID NO: 47 - DNA, 45 bp, Artificial Sequence
  D15111
gctccggctttgacttggttcacgtgtgaccctcttgcagcaacc

SEQ ID NO: 48 - DNA, 54 bp, Artificial Sequence
  D15112
ttccacggtgtgtccactccttaatttgcctgattatgcttccatcaaaatttg

SEQ ID NO: 49 - DNA, 43 bp, Artificial Sequence
  D15113
caggaaacagctatgaccatgattatatccgtcacggtggctc

SEQ ID NO: 50 - DNA, 22 bp, Artificial Sequence
  D39394
taggtagtattggggcatcagg

SEQ ID NO: 51 - DNA, 27 bp, Artificial Sequence
  D39395
taatctcgatgtgatgattgctgaagg

SEQ ID NO: 52 - DNA, 35 bp, Artificial Sequence
  D99047654
attaaggagtggacacaccgtggaaacggatgaag

SEQ ID NO: 53 - DNA, 32 bp, Artificial Sequence
  D99047655
acgtgaaccaagtcaaagccggagcgcttttg

SEQ ID NO: 54 - DNA, 33 bp, Artificial Sequence
  D98646038
attaaggagtggacaactggaaacggatgaagg

SEQ ID NO: 55 - DNA, 36 bp, Artificial Sequence
  D98646039
acgtgaaccaagtcatgctttttaagggaattgtgc

SEQ ID NO: 56 - protein, 474 aa, Artificial Sequence
  TriA original
MQTLSIQHGTLVTMDQYRRVLGDSWVHVQDGRIVALGVHAESVPPPADRVIDARGKVVLPGFINAHTHVNQILLRGGPSHGRQLYDWLFNVLYPGQKAMRPEDVAVAVRLYCAEAVRSGITTINDNADSAIYPGNIEAAMAVYGEVGVRVVYARMFFDRMDGRIQGYVDALKARSPQVELCSIMEETAVAKDRITALSDQYHGTAGGRISVWPAPAITPAVTVEGMRWAQAFARDRAVMWTLHMAESDHDERLHWMSPAEYMECYGLLDERLQVAHCVYFDRKDVRLLHRHNVKVASQVVSNAYLGSGVAPVPEMVERGMAVGIGTDDGNCNDSVNMIGDMKFMAHIHRAVHRDADVLTPEKILEMATIDGARSLGMDHEIGSIETGKRADLILLDLRHPQTTPHHHLAATIVFQAYGNEVDTVLIDGNVVMENRRLSFLPPERELAFLEEAQSRATAILQRANMVANPAWRSL

SEQ ID NO: 57 - DNA, 1425 bp, Artificial Sequence
  Acidovorax avenae subsp. citrulli NRRL B-12227
atgcaaacgctcagcatccagcacggtaccctcgtcacgatggatcagtaccgcagagtccttggggatagctgggttcacgtgcaggatggacggatcgtcgcgctcggagtgcacgccgagtcggtgcctccgccagcggatcgggtgatcgatgcacgcggcaaggtcgtgttacccggtttcatcaatgcccacacccatgtgaaccagatcctcctgcgcggagggccctcgcacgggcgtcaactctatgactggctgttcaacgttttgtatccgggacaaaaggcgatgagaccggaggacgtagcggtggcggtgaggttgtattgtgcggaagctgtgcgcagcgggattacgacgatcaacgacaacgccgattcggccatctacccaggcaacatcgaggccgcgatggcggtctatggtgaggtgggtgtgagggtcgtctacgcccgcatgttctttgatcggatggacgggcgcattcaagggtatgtggacgccttgaaggctcgctctccccaagtcgaactgtgctcgatcatggaggaaacggctgtggccaaagatcggatcacagccctgtcagatcagtatcatggcacggcaggaggtcgtatatcagtttggcccgctcctgccattaccccggcggtgacagttgaaggaatgcgatgggcacaagccttcgcccgtgatcgggcggtaatgtggacgcttcacatggcggagagcgatcatgatgagcggcttcattggatgagtcccgccgagtacatggagtgttacggactcttggatgagcgtctgcaggtcgcgcattgcgtgtactttgaccggaaggatgttcggctgctgcaccgccacaatgtgaaggtcgcgtcgcaggttgtgagcaatgcctacctcggctcaggggtggcccccgtgccagagatggtggagcgcggcatggccgtgggcattggaacagatgacgggaattgtaatgactccgtaaacatgatcggagacatgaagtttatggcccatattcaccgcgcggtgcatcgggatgcggacgtgctgaccccagagaagattcttgaaatggcgacgatcgatggggcgcgttcgttgggaatggaccacgagattggttccatcgaaaccggcaagcgcgcggaccttatcctgcttgacctgcgtcaccctcagacgactcctcaccatcatttggcggccacgatcgtgtttcaggcttacggcaatgaggtggacactgtcctgattgacggaaacgttgtgatggagaaccgccgcttgagctttcttccccctgaacgtgagttggcgttccttgaggaagcgcagagccgcgccacagctattttgcagcgggcgaacatggtggctaacccagcttggcgcagcctctag

SEQ ID NO: 58 - protein, 470 aa, Artificial Sequence
  Mel1 TriA
MQTLSIQHGTLVTMDQYRRVLGDSWVHVQDGRIVALGVHAESVPPPADRVIDARGKVVLPGFINAHTHVNQILLRGGPSHGRQLYDWLFNVLYPGQKAMRPEDVAVAVRLYCAEAVRSGITTINDNADSAIYPGNIEAAMAVYGEVGVRVVYARMFFDRMDGRIQGYVDALKARSPQVELCSIMEETAVAKDRITALSDQYHGTAGGRISVWPAPAITPAVTVEGMRWAQAFARDRAVMWTLHMAESDHDERLHWMSPAEYMECYGLLDERLQVAHCVYFDRKDVRLLHRHNVKVASQVVSNAYLGSGVAPVPEMVERGMAVGIGTDDGNCNDSVNMIGDMKFMAHIHRAVHRDADVLTPEKILEMATIDGARSLGMDHEIGSIETGKRADLILLDLRHPQTTPHHHLAATIVFQAYGNEVDTVLIDGNVVMENRRLSFLPPERELAFLEEAQSRATAILQRANMVANPA

SEQ ID NO: 59 - DNA, 1443 bp, Artificial Sequence
  Mel1 gene sequence
  RBS (5)..(10)
  gene (19)..(1443)
agttaggagattcagaccatgcaaaccctcagcattcaacatggcaccctcgtgacgatggatcaatatcgccgggtgctcggcgatagctgggtgcatgtgcaagatggccgcattgtggccctcggtgtgcatgccgaatctgtgcccccccccgccgatcgtgtgattgatgcccgcggtaaagtggtgctccccggttttattaatgcccatacccacgtgaatcaaattctcctccgtggtggtccctctcacggtcgccaactctatgattggctctttaatgtgctctaccccggccaaaaagccatgcgccccgaagatgtggccgtggccgtgcggctctattgtgccgaagccgtgcgcagtggtattaccaccattaatgataatgccgattccgccatttaccccggcaatattgaagccgcgatggccgtgtatggcgaagtgggtgtgcgggtggtgtacgcccgcatgtttttcgatcgcatggatggccggattcaaggttatgtggatgccctcaaagcccggagcccccaagtggaactctgttctattatggaagaaaccgccgtggccaaagatcggattaccgccctcagcgatcaatatcacggcaccgccggtggccgcattagtgtgtggcccgcccccgccattacccccgccgtgaccgtggagggtatgcgttgggcccaagcctttgcccgcgatcgggccgtgatgtggaccctccacatggccgaaagcgatcatgatgaacggctccactggatgtctcccgccgaatatatggaatgttacggcctcctcgatgaacgcctccaagtggcccactgtgtgtattttgatcgcaaagatgtgcggctcctccatcgccacaatgtgaaagtggccagtcaagtggtgtccaatgcctacctcggcagtggtgtggcccccgtgcccgaaatggtggaacgtggcatggccgtgggcattggcaccgatgatggtaattgtaatgattccgtgaatatgattggcgatatgaaatttatggcccatattcaccgggccgtgcatcgcgatgccgatgtgctcacccccgaaaaaattctcgaaatggccaccattgatggcgcccgcagtctcggtatggatcatgaaattggctccattgaaaccggtaaacgggccgatctcattctcctcgatctccgccacccccaaaccaccccccaccatcacctcgccgccaccattgtgtttcaagcctacggtaatgaagtggataccgtgctcattgatggcaatgtggtgatggaaaatcgccggctcagttttctcccccccgaacgggaactcgcctttctcgaagaagcccaaagtcgcgccaccgccattctccaacgcgccaatatggtggccaatcccgcctgacgcagcctctaa

SEQ ID NO: 60 - protein, 474 aa, Artificial Sequence
  Mel4 TriA.
MQTLSIQHGTLVTMDQYRRVLGDSWVHVQDGRIVALGVHAESVPPPADRVIDARGKVVLPGFINAHTHVNQILLRGGPSHGRQLYDWFFNVLYPGQKAMRPEDVAVAVRLYCAEAVRSGITTINDNADSAIYPGNIEAAMAVYGEVGVRVVYARMFFDRMDGRIQGYVDALKARSPQVELCSIMEETAVAKDRITALSDQYHGTAGGRISVWPAPAITPAVTVEGMRWAQAFARDRAVMWTLHMAESDHDERLHWMSPAEYMECYGLLDERLQVAHCVYFDRKDVRLLHRHNVKVASQVVSNAYLGSGVAPVPEMVERGMAVGIGTDDGNCNDSVNMIGDMKFMAHIHRAVHRDADVLTPEKILEMATIDGARSLGMDHEIGSIETGKRADLILLDLRHPQTTPHHHLAATIVFQAYGNEVDTVLIDGNVVMENRRLSFLPPERELAFLEEAQSRATAILQRANMVANPAWRSL

SEQ ID NO: 61 - DNA, 1443 bp, Artificial Sequence
  Mel4 gene sequence
  RBS (5)..(10)
  gene (19)..(1443): triA gene sequence
agttaggagattcagaccatgcaaaccctcagcattcaacatggcaccctcgtgacgatggatcaatatcgccgggtgctcggcgatagctgggtgcatgtgcaagatggccgcattgtggccctcggtgtgcatgccgaatctgtgcccccccccgccgatcgtgtgattgatgcccgcggtaaagtggtgctccccggttttattaatgcccatacccacgtgaatcaaattctcctccgtggtggtccctctcacggtcgccaactctatgattggttctttaatgtgctctaccccggccaaaaagccatgcgccccgaagatgtggccgtggccgtgcggctctattgtgccgaagccgtgcgcagtggtattaccaccattaatgataatgccgattccgccatttaccccggcaatattgaagccgcgatggccgtgtatggcgaagtgggtgtgcgggtggtgtacgcccgcatgtttttcgatcgcatggatggccggattcaaggttatgtggatgccctcaaagcccggagcccccaagtggaactctgttctattatggaagaaaccgccgtggccaaagatcggattaccgccctcagcgatcaatatcacggcaccgccggtggccgcattagtgtgtggcccgcccccgccattacccccgccgtgaccgtggagggtatgcgttgggcccaagcctttgcccgcgatcgggccgtgatgtggaccctccacatggccgaaagcgatcatgatgaacggctccactggatgtctcccgccgaatatatggaatgttacggcctcctcgatgaacgcctccaagtggcccactgtgtgtattttgatcgcaaagatgtgcggctcctccatcgccacaatgtgaaagtggccagtcaagtggtgtccaatgcctacctcggcagtggtgtggcccccgtgcccgaaatggtggaacgtggcatggccgtgggcattggcaccgatgatggtaattgtaatgattccgtgaatatgattggcgatatgaaatttatggcccatattcaccgggccgtgcatcgcgatgccgatgtgctcacccccgaaaaaattctcgaaatggccaccattgatggcgcccgcagtctcggtatggatcatgaaattggctccattgaaaccggtaaacgggccgatctcattctcctcgatctccgccacccccaaaccaccccccaccatcacctcgccgccaccattgtgtttcaagcctacggtaatgaagtggataccgtgctcattgatggcaatgtggtgatggaaaatcgccggctcagttttctcccccccgaacgggaactcgcctttctcgaagaagcccaaagtcgcgccaccgccattctccaacgcgccaatatggtggccaatcccgcctggcgcagcctctaa

SEQ ID NO: 62 - protein, 474 aa, Artificial Sequence
  Mel6 TriA
MQTLSIQHGTLVTMDQYRRVLGDSWVHVQDGRIVALGVHAESVPPPADRVIDARGKVVLPGFINAHTHVNQILLRGGPSHGRQLYDWLFNVLYPGQKAMRPEDVAVAVRLYCAEAVRSGITTINDNADSAIYPGNIEAAMAVYGEVGVRVVYARMFFDRMDGRIQGYVDALKARSPQVELCSIMEETAVAKDRITALSDQYHGTAGGRISVWPAPAITPAVTVEGMRWAQAFARDRAVMWTLHMAESDHDERLHWMSPAEYMECYGLLDERLQVAHCVYFDRKDVRLLHRHNVKVASQVVSNAYLGSGVAPVPEMVKRGMAVGIGTDDGNCNDSVNMIGDMKFMAHIHRAVHRDADVLTPEKILEMATIDGARSLGMDHEIGSIETGKRADLILLDLRHPQTTPHHHLAATIVFQAYGNEVDTVLIDGNVVMENRRLSFLPPERELAFLEEAQSRATAILQRANMVANPAWRSL

SEQ ID NO: 63 - DNA, 1443 bp, Artificial Sequence
  Mel6 gene sequence
  RBS (5)..(10)
  gene (19)..(1443): triA gene sequence
agttaggagattcagaccatgcaaaccctcagcattcaacatggcaccctcgtgacgatggatcaatatcgccgggtgctcggcgatagctgggtgcatgtgcaagatggccgcattgtggccctcggtgtgcatgccgaatctgtgcccccccccgccgatcgtgtgattgatgcccgcggtaaagtggtgctccccggttttattaatgcccatacccacgtgaatcaaattctcctccgtggtggtccctctcacggtcgccaactctatgattggctctttaatgtgctctaccccggccaaaaagccatgcgccccgaagatgtggccgtggccgtgcggctctattgtgccgaagccgtgcgcagtggtattaccaccattaatgataatgccgattccgccatttaccccggcaatattgaagccgcgatggccgtgtatggcgaagtgggtgtgcgggtggtgtacgcccgcatgtttttcgatcgcatggatggccggattcaaggttatgtggatgccctcaaagcccggagcccccaagtggaactctgttctattatggaagaaaccgccgtggccaaagatcggattaccgccctcagcgatcaatatcacggcaccgccggtggccgcattagtgtgtggcccgcccccgccattacccccgccgtgaccgtggagggtatgcgttgggcccaagcctttgcccgcgatcgggccgtgatgtggaccctccacatggccgaaagcgatcatgatgaacggctccactggatgtctcccgccgaatatatggaatgttacggcctcctcgatgaacgcctccaagtggcccactgtgtgtattttgatcgcaaagatgtgcggctcctccatcgccacaatgtgaaagtggccagtcaagtggtgtccaatgcctacctcggcagtggtgtggcccccgtgcccgaaatggtgaaacgtggcatggccgtgggcattggcaccgatgatggtaattgtaatgattccgtgaatatgattggcgatatgaaatttatggcccatattcaccgggccgtgcatcgcgatgccgatgtgctcacccccgaaaaaattctcgaaatggccaccattgatggcgcccgcagtctcggtatggatcatgaaattggctccattgaaaccggtaaacgggccgatctcattctcctcgatctccgccacccccaaaccaccccccaccatcacctcgccgccaccattgtgtttcaagcctacggtaatgaagtggataccgtgctcattgatggcaatgtggtgatggaaaatcgccggctcagttttctcccccccgaacgggaactcgcctttctcgaagaagcccaaagtcgcgccaccgccattctccaacgcgccaatatggtggccaatcccgcctggcgcagcctctaa

SEQ ID NO: 64 - protein, 474 aa, Artificial Sequence
  Mel7 TriA
MQTLSIQHGTLVTMDQYRRVLGDSWVHVQDGRIVALGVHAESVPPPADRVIDARGKVVLPGFINAHTHVNQILLRGGPSHGRQLYDWLFNVLYPGQKAMRPEDVAVAVRLYCAEAVRSGITTINDNADSAIYPGNIEAAMAVYGEVGVRVVYARMFFDRMDGRIQGYVDALKARSPQVELCSIMEETAVAKDRITALSDQYHGTAGGRISVWPAPAITPAVTVEGMRWAQAFARDRAVMWTLHMAESDHDERLYWMSPAEYMECYGLLDERLQVAHCVYFDRKDVRLLHRHNVKVASQVVSNAYLGSGVAPVPEMVERGMAVGIGTDDGNCNDSVNMIGDMKFMAHIHRAVHRDADVLTPEKILEMATIDGARSLGMDHEIGSIETGKRADLILLDLRHPQTTPHHHLAATIVFQAYGNEVDTVLIDGNVVMENRRLSFLPPERELAFLEEAQSRATAILQRANMVANPAWRSL

SEQ ID NO: 65 - DNA, 1443 bp, Artificial Sequence
  Mel7 gene sequence
  RBS (5)..(10)
  gene (19)..(1443): triA gene sequence
agttaggagattcagaccatgcaaaccctcagcattcaacatggcaccctcgtgacgatggatcaatatcgccgggtgctcggcgatagctgggtgcatgtgcaagatggccgcattgtggccctcggtgtgcatgccgaatctgtgcccccccccgccgatcgtgtgattgatgcccgcggtaaagtggtgctccccggttttattaatgcccatacccacgtgaatcaaattctcctccgtggtggtccctctcacggtcgccaactctatgattggctctttaatgtgctctaccccggccaaaaagccatgcgccccgaagatgtggccgtggccgtgcggctctattgtgccgaagccgtgcgcagtggtattaccaccattaatgataatgccgattccgccatttaccccggcaatattgaagccgcgatggccgtgtatggcgaagtgggtgtgcgggtggtgtacgcccgcatgtttttcgatcgcatggatggccggattcaaggttatgtggatgccctcaaagcccggagcccccaagtggaactctgttctattatggaagaaaccgccgtggccaaagatcggattaccgccctcagcgatcaatatcacggcaccgccggtggccgcattagtgtgtggcccgcccccgccattacccccgccgtgaccgtggagggtatgcgttgggcccaagcctttgcccgcgatcgggccgtgatgtggaccctccacatggccgaaagcgatcatgatgaacggctctactggatgtctcccgccgaatatatggaatgttacggcctcctcgatgaacgcctccaagtggcccactgtgtgtattttgatcgcaaagatgtgcggctcctccatcgccacaatgtgaaagtggccagtcaagtggtgtccaatgcctacctcggcagtggtgtggcccccgtgcccgaaatggtggaacgtggcatggccgtgggcattggcaccgatgatggtaattgtaatgattccgtgaatatgattggcgatatgaaatttatggcccatattcaccgggccgtgcatcgcgatgccgatgtgctcacccccgaaaaaattctcgaaatggccaccattgatggcgcccgcagtctcggtatggatcatgaaattggctccattgaaaccggtaaacgggccgatctcattctcctcgatctccgccacccccaaaccaccccccaccatcacctcgccgccaccattgtgtttcaagcctacggtaatgaagtggataccgtgctcattgatggcaatgtggtgatggaaaatcgccggctcagttttctcccccccgaacgggaactcgcctttctcgaagaagcccaaagtcgcgccaccgccattctccaacgcgccaatatggtggccaatcccgcctggcgcagcctctaa

SEQ ID NO: 66 - protein, 474 aa, Artificial Sequence
  Mel8 TriA
MQTLSIQHGTLVTMDQYRRVLGDSWVHVQDGRIVALGVHAESVPPPADRVIDARGKVVLPGFINAHTHVNQILLRGGPSHGRQLYDWLFNVLYPGQKAMRPEDVAVAVRLYCAEAVRSGITTINDNADSAIYPGNIEAAMAVYGEVGVRVVYARMFFDRMDGRIQGYVDALKARSPQVELCSIMEETAVAKDRITALSDQYHGTAGGRISVWPAPAITPAVTVEGMRWAQAFARDRAVMWTLHMAESDHDERLHWMSPAEYMECYGLLDERLQVAHCVYFDRKDVRLLHRHNVKVASQVVSNAYLGSGVAPVPEMVERGMAVGIGTDDGNCNDSVNMIGDMKFMAHIHRAVHRDVDVLTPEKILEMATIDGARSLGMDHEIGSIETGKRADLILLDLRHPQTTPHHHLAATIVFQAYGNEVDTVLIDGNVVMENRRLSFLPPERELAFLEEAQSRATAILQRANMVANPAWRSL

SEQ ID NO: 67 - DNA, 1443 bp, Artificial Sequence
  Mel8 gene sequence
  RBS (5)..(10)
  gene (19)..(1443): triA gene sequence
agttaggagattcagaccatgcaaaccctcagcattcaacatggcaccctcgtgacgatggatcaatatcgccgggtgctcggcgatagctgggtgcatgtgcaagatggccgcattgtggccctcggtgtgcatgccgaatctgtgcccccccccgccgatcgtgtgattgatgcccgcggtaaagtggtgctccccggttttattaatgcccatacccacgtgaatcaaattctcctccgtggtggtccctctcacggtcgccaactctatgattggctctttaatgtgctctaccccggccaaaaagccatgcgccccgaagatgtggccgtggccgtgcggctctattgtgccgaagccgtgcgcagtggtattaccaccattaatgataatgccgattccgccatttaccccggcaatattgaagccgcgatggccgtgtatggcgaagtgggtgtgcgggtggtgtacgcccgcatgtttttcgatcgcatggatggccggattcaaggttatgtggatgccctcaaagcccggagcccccaagtggaactctgttctattatggaagaaaccgccgtggccaaagatcggattaccgccctcagcgatcaatatcacggcaccgccggtggccgcattagtgtgtggcccgcccccgccattacccccgccgtgaccgtggagggtatgcgttgggcccaagcctttgcccgcgatcgggccgtgatgtggaccctccacatggccgaaagcgatcatgatgaacggctccactggatgtctcccgccgaatatatggaatgttacggcctcctcgatgaacgcctccaagtggcccactgtgtgtattttgatcgcaaagatgtgcggctcctccatcgccacaatgtgaaagtggccagtcaagtggtgtccaatgcctacctcggcagtggtgtggcccccgtgcccgaaatggtggaacgtggcatggccgtgggcattggcaccgatgatggtaattgtaatgattccgtgaatatgattggcgatatgaaatttatggcccatattcaccgggccgtgcatcgcgatgtcgatgtgctcacccccgaaaaaattctcgaaatggccaccattgatggcgcccgcagtctcggtatggatcatgaaattggctccattgaaaccggtaaacgggccgatctcattctcctcgatctccgccacccccaaaccaccccccaccatcacctcgccgccaccattgtgtttcaagcctacggtaatgaagtggataccgtgctcattgatggcaatgtggtgatggaaaatcgccggctcagttttctcccccccgaacgggaactcgcctttctcgaagaagcccaaagtcgcgccaccgccattctccaacgcgccaatatggtggccaatcccgcctggcgcagcctctaa

SEQ ID NO: 68 - protein, 474 aa, Artificial Sequence
  Mel5Evo TriA
MQTLSIQHGTLVTMDQYRRVLGDSWVHVQDGRIVALGVHAESVPPPADRVIDARGKVVLPGFINAHTHVNQILLRGGPSHGRQLYDWLFNVLYPGQKAMRPEDVAVAVRLYCAEAVRSGITTINDNADSAIYPGNIEAAMAVYGEVGVRVVYARMFFDRMDGRIQGYVDALKARSPQVELCSIMEETAVAKDRITALSDQYHGTAGGRISVWPAPAINPAVTVEGMRWAQAFARDRAVMWTLHMAESDHDERLHWMSPAEYMECYGLLDERLQVAHCMYFDRKDVRLLHRHNVKVASQVVSNAYLGSGVAPVPEMVERGMAVGIGTDDGNCNDSVNMIGDMKFMAHIHRAVHRDADVLTPEKILEMATIDGARSLGMDHEIGSIETGKRADLILLDLRHPQTTPHHHLAATIVFQAYGNEVDTVLIDGNVVMENRRLSFLPPERELAFLEEAQSRATAILQRANMVANPAWRSL

SEQ ID NO: 69 - DNA, 1446 bp, Artificial Sequence
  Mel5Evo
  RBS (1)..(21)
  gene (22)..(1446): triA gene sequence
gctagttagaagattcagaccatgcaaaccctcagcattcaacatggcaccctcgtgacgatggatcaatatcgccgggtgctcggcgatagctgggtgcatgtgcaagatggccgcattgtggccctcggtgtgcatgccgaatctgtgcccccccccgccgatcgtgtgattgatgcccgcggtaaagtggtgctccccggttttattaatgcccatacccacgtgaatcaaattctcctccgtggtggtccctctcacggtcgccaactctatgattggctctttaatgtgctctaccccggccaaaaagccatgcgccccgaagatgtggccgtggccgtgcggctctattgtgccgaagccgtgcgcagtggtattaccaccattaatgataatgccgattccgccatttaccccggcaatattgaagccgcgatggccgtgtatggcgaagtgggtgtgcgggtggtgtacgcccgcatgtttttcgatcgcatggatggccggattcaaggttatgtggatgccctcaaagcccggagcccccaagtggaactctgttctattatggaagaaaccgccgtggccaaagatcggattaccgccctcagcgatcaatatcacggcaccgccggtggccgcattagtgtgtggcccgcccccgccattaaccccgccgtgaccgtggagggtatgcgttgggcccaagcctttgcccgcgatcgggccgtgatgtggaccctccacatggccgaaagcgatcatgatgaacggctccactggatgtctcccgccgaatatatggaatgttacggcctcctcgatgaacgcctccaagtggcccactgtatgtattttgatcgcaaagatgtgcggctcctccatcgccacaatgtgaaagtggccagtcaagtggtgtccaatgcctacctcggcagtggtgtggcccccgtgcccgaaatggtggaacgtggcatggccgtgggcattggcaccgatgatggtaattgtaatgattccgtgaatatgattggcgatatgaaatttatggcccatattcaccgggccgtgcatcgcgatgccgatgtgctcacccccgaaaaaattctcgaaatggccaccattgatggcgcccgcagtctcggtatggatcatgaaattggctccattgaaaccggtaaacgggccgatctcattctcctcgatctccgccacccccaaaccaccccccaccatcacctcgccgccaccattgtgtttcaagcctacggtaatgaagtggataccgtgctcattgatggcaatgtggtgatggaaaatcgccggctcagttttctcccccccgaacgggaactcgcctttctcgaagaagcccaaagtcgcgccaccgccattctccaacgcgccaatatggtggccaatcccgcctggcgcagcctctaa

SEQ ID NO: 70 - DNA, 1443 bp, Artificial Sequence
  Mel5 triA including RBS
  RBS (5)..(10)
  gene (19)..(1443): triA gene sequence
agttagaagattcagaccatgcaaaccctcagcattcaacatggcaccctcgtgacgatggatcaatatcgccgggtgctcggcgatagctgggtgcatgtgcaagatggccgcattgtggccctcggtgtgcatgccgaatctgtgcccccccccgccgatcgtgtgattgatgcccgcggtaaagtggtgctccccggttttattaatgcccatacccacgtgaatcaaattctcctccgtggtggtccctctcacggtcgccaactctatgattggctctttaatgtgctctaccccggccaaaaagccatgcgccccgaagatgtggccgtggccgtgcggctctattgtgccgaagccgtgcgcagtggtattaccaccattaatgataatgccgattccgccatttaccccggcaatattgaagccgcgatggccgtgtatggcgaagtgggtgtgcgggtggtgtacgcccgcatgtttttcgatcgcatggatggccggattcaaggttatgtggatgccctcaaagcccggagcccccaagtggaactctgttctattatggaagaaaccgccgtggccaaagatcggattaccgccctcagcgatcaatatcacggcaccgccggtggccgcattagtgtgtggcccgcccccgccattacccccgccgtgaccgtggagggtatgcgttgggcccaagcctttgcccgcgatcgggccgtgatgtggaccctccacatggccgaaagcgatcatgatgaacggctccactggatgtctcccgccgaatatatggaatgttacggcctcctcgatgaacgcctccaagtggcccactgtgtgtattttgatcgcaaagatgtgcggctcctccatcgccacaatgtgaaagtggccagtcaagtggtgtccaatgcctacctcggcagtggtgtggcccccgtgcccgaaatggtggaacgtggcatggccgtgggcattggcaccgatgatggtaattgtaatgattccgtgaatatgattggcgatatgaaatttatggcccatattcaccgggccgtgcatcgcgatgccgatgtgctcacccccgaaaaaattctcgaaatggccaccattgatggcgcccgcagtctcggtatggatcatgaaattggctccattgaaaccggtaaacgggccgatctcattctcctcgatctccgccacccccaaaccaccccccaccatcacctcgccgccaccattgtgtttcaagcctacggtaatgaagtggataccgtgctcattgatggcaatgtggtgatggaaaatcgccggctcagttttctcccccccgaacgggaactcgcctttctcgaagaagcccaaagtcgcgccaccgccattctccaacgcgccaatatggtggccaatcccgcctggcgcagcctctaa

SEQ ID NO: 71 - DNA, 726 bp, Artificial Sequence
  trzE Rhodococcus sp. Mel
atgatatactcaacagtcaacgctaatccttacgcttggccttacgatggttcaatagaccctgctcacaccgctttaatcttaatcgattggcaaatagacttttgtggtccaggtggttatgtcgattccatgggttacgacttatccttgactagaagtggtttagaacctacagcaagagtattggctgcagccagagatactggtatgacagttatccatactagagaaggtcacagaccagatttggctgacttgccacctaataagagatggagatctgcatcagccggtgctgaaatcggttcagttggtccatgtggtagaattttagtcagaggtgaacctggttgggaaatagtaccagaagttgcacctagagaaggtgaaccaattatagataaacctggtaaaggtgctttctacgcaacagatttggacttgttgttgagaacaagaggtatcacccatttgattttgaccggtataactacagatgtttgcgtccacaccactatgagagaagccaacgatagaggttacgaatgtttaattttgtctgattgcaccggtgctactgacagaaagcatcacgaagctgcattatctatggtcaccatgcaaggtggtgtattcggtgcaactgcccattcagatgacttattggccgctttgggtacaaccgttccagcagccgctggtcctagagctagaacagaataa

SEQ ID NO: 72 - DNA, 726 bp, Artificial Sequence
  trzE Rhodococcus sp. Mel
atgatttatagtaccgtgaatgccaatccctatgcctggccctacgatggctccattgatcccgcccacaccgccctcatcctcatcgattggcaaatcgatttttgtggtcccggcggttatgtggatagtatgggctacgatctcagcctcacccgctctggcctcgaacccaccgcccgcgtgctcgccgccgcccgcgataccggcatgaccgtgattcacacccgcgaaggtcatcgtcccgatctcgccgatctcccccccaataaacgttggcggagtgcctccgccggcgccgaaattggcagtgtgggtccctgtggccgcattctcgtgcgtggtgaacccggttgggaaattgtgcccgaagtggccccccgcgaaggtgaacccattattgataaacccggtaaaggtgccttttatgccaccgatctcgatctcctcctccgcacccgcggcattacccacctcattctcaccggcattaccaccgatgtgtgtgtgcataccaccatgcgcgaagccaatgatcggggctacgaatgtctcattctctccgattgtaccggtgccaccgatcggaaacaccatgaagccgccctcagcatggtgaccatgcaaggcggtgtgtttggtgccaccgcccattctgatgatctcctcgccgccctcggcaccaccgtgcccgccgccgccggcccccgtgcccgcaccgaataa

SEQ ID NO: 73 - DNA, 1239 bp, Artificial Sequence
  trzC Acidovorax avenae subsp. citrulli NRRL B-12227
atgtcaatggaaacccatagttatgtagacgtcgcaattcgtaacgcgcgtcttgccgatacggagggaattgtcgatattcttattcacgatgggcgcattgcgtccatcgtgaagtcgacaaaaacaaaaggatcggtggagatcgatgctcatgagggtctggtcacttccggcctggtagagcctcacatccatctcgataaggccctgacggcagatcgggttcccgcaggaagcattggcgaccttcgaacgcgacgaggccttgagatggcaattcgggccacccgtgatatcaagcgtacgttcacggttgaagatgttcgagaacgggccatacgtgcggccctgatggcatcccgtgcgggaaccaccgcattgcggacacacgtcgatgtcgacccgattgtcggcctcgcaggtatccgtggtgtccttgaggcgcgtgaagtctgcgcgggattgatcgatatccagatcgtcgccttccctcaggagggactcttctgctctgcgggggccgtggacctcatgcgggaggcgatcaaactgggcgcggatgccgtcggcggcgcacccgcgctggatgatcgcccgcaggaccatgtccgagccgtttttgaccttgctgctgagttcggcctgcccgtagacatgcacgtcgatgagtccgaccggcgggaagactttacgcttccctttgtgattgaagctgcccgtgaacggcgtgtgcccaatgtgaccgtcgcgcacatcagctcgctgtccgtacagacggatgacgtagcacggtcgaccattgccgcccttgcggacgccgatgttaatgtcgtggttaatccgatcattgtcaaaattacgcggctgagtgaattactcgatgccggagtctccgtaatgtttggctcggacaacctgcgggatccgttctatccgctcggagcggcgaatccccttggatcagccatttttgcctgtcaaattgccgcgctgggaacaccgcaagatctcagacgggtattcgatgcggtcaccatcaacgctgcccgcatgctgggattcccctcacttttaggcgtcgtggaaggggcagtcgcggatctcgcagtattcccatcggcgacgcccgaggaggttgttctggatcaacagtctccgctcttcgtactcaagggcggacgtgtcgttgccatgcgattggccgctggatcaacgtcgttccgcgactactcatga

SEQ ID NO: 74 - DNA, 1239 bp, Artificial Sequence
  trzC Acidovorax avenae subsp. citrulli NRRL B-12227
atgagcatggaaacccactcctatgttgatgttgcgatccgtaacgcccgtctcgccgataccgaaggcattgttgatattctgattcatgatggtcggattgccagcattgtgaaatccaccaaaaccaaaggttccgtggaaattgatgcccatgaaggtctcgtgaccagtggcctcgtggaaccccacattcatctcgataaagccctcaccgccgatcgcgtgcccgccggtagtattggcgatctccggacccgccggggtctcgaaatggccattcgcgccacccgcgatattaaacgcacctttaccgtggaagatgtgcgggaacgcgccattcgggccgccctcatggccagtcgcgccggcaccaccgccctccgcacccatgtggatgtggaccccattgtgggtctcgccggtattcggggcgtgctcgaagcccgcgaagtgtgtgccggtctcattgatattcaaattgtggcctttccccaagaaggcctcttttgtagtgccggtgccgtggatctcatgcgggaagccattaaactcggtgccgatgccgtgggcggtgcccccgccctcgatgatcgcccccaagatcatgtgcgcgccgtgtttgatctcgccgccgaatttggtctccccgtggatatgcacgtggatgaaagtgatcgccgggaagattttaccctcccctttgtgattgaagccgcccgcgaacggcgcgtgcccaatgtgaccgtggcccacatttcctctctcagtgtgcaaaccgatgatgtggcccgctccaccattgccgccctcgccgatgccgatgtgaatgtggtggtgaatcccattattgtgaaaattacccgcctctctgaactcctcgatgccggtgtgtctgtgatgtttggctccgataatctccgcgatcccttttatcccctcggtgccgccaatcccctcggtagtgccatttttgcctgtcaaattgccgccctcggcaccccccaagacctccgccgggtgtttgatgccgtgaccattaatgccgcccgcatgctcggttttcccagtctcctcggtgtggtggaaggtgccgtggccgatctcgccgtgtttcccagtgccacccccgaagaagtggtgctcgatcaacaaagtcccctctttgtgctcaaaggtggtcgggtcgttgcgatgcggttagcggcgggtagcacctcctttcgtgattattcgtaa

SEQ ID NO: 75 - DNA, 1866 bp, Artificial Sequence
  DUR 1,2 Saccharomyces cerevisiae, truncation amino acids 1-622
atgacagttagttccgatacaactgctgaaatatcgttaggttggtcaatccaagactggattgatttccacaagtcatcaagctcccaggcttcactaaggcttcttgaatcactactagactctcaaaatgttgcgccagtcgataatgcgtggatatcgctaatttcaaaggaaaatttactgcaccaattccaaattttaaagagcagagaaaataaagaaactctacctctctacggtgtccctattgctgttaaggacaacatcgacgttagaggtctacccaccaccgctgcatgtccatcctttgcatatgagccttccaaagactctaaagtagtagaactactaagaaatgcaggtgcgataatcgtgggtaagacaaacttggaccaatttgccacaggattagtcggcacacggtctccatatgggaaaacaccttgcgcttttagcaaagagcatgtatctggtggttcctccgctgggtcagcatcggtggtcgccagaggtatcgtaccaattgcattgggtactgatacagcaggttctggtagagtcccagccgccttgaacaacctgattggcctaaagccaacaaagggcgtcttttcctgtcaaggtgtagttcccgcttgtaaatctttagactgcgtctccatctttgcattaaacctaagtgatgctgaacgctgcttccgcatcatgtgccagccagatcctgataatgatgaatattctagaccctatgtttccaaccctttgaaaaaattttcaagcaatgtaacgattgctattcctaaaaatatcccatggtatggtgaaaccaagaatcctgtactgttttccaatgctgtcgaaaatctatcaagaacgggcgctaacgtcatagaaattgattttgagcctcttttagagttagctcgctgtttatacgaaggtacttgggtggccgagcgttatcaagctattcaatcgtttttggacagtaaaccaccaaaggaatctttggaccctactgttatttcaattatagaaggggccaagaaatacagtgcagtagactgcttcagttttgaatacaaaagacaaggcatcttgcaaaaagtgagacgacttctcgaatcagtcgatgtattgtgtgtgcccacatgtcctttaaatcctactatgcaacaagttgcggatgaaccagtcctagtcaattcaagacaaggcacatggactaattttgtcaacttggcagatttggcagcccttgctgttcccgcagggttccgagacgatggtttgccaaatggtattactttaatcggtaaaaaattcacagattacgcactattagagttggctaaccgctatttccaaaatatattccccaacggttccagaacatacggtacttttacctcttcttcagtaaagccagcaaacgatcaattagtgggaccagactatgacccatctacgtccataaaattggctgttgtcggtgcacatcttaagggtctgcctctacattggcaattggaaaaggtcaatgcaacatatttatgtacaacaaaaacatcaaaagcttaccagctttttgctttgcccaaaaatggaccagttttaaaacctggtttgagaagagttcaagatagcaatggctctcaaatcgaattagaagtgtacagtgttccaaaagaactgttcggtgcttttatttccatggttcctgaaccattaggaataggttcagtggagttagaatctggtgaatggatcaaatcctttatttgtgaagaatctggttacaaagccaaaggtacagttgatatcacaaagtatggtggatttagagcatattttgaaatgttg

SEQ ID NO: 76 - DNA, 1869 bp, Artificial Sequence
  DUR 1,2 Saccharomyces cerevisiae, truncation amino acids 1-622
atgaccgtgagttccgataccaccgccgaaatttctctcggctggagtatccaagattggatcgattttcacaaaagctctagttcccaagcctccctccgcctcctcgaatccctcctcgatagccaaaatgtggcccccgtggataatgcctggatttccctcattagcaaggaaaacctcctccatcaatttcaaattctcaagtctcgcgaaaacaaggaaaccctccccctctatggtgtgcccattgccgtgaaagataatattgatgtgcgtggcctccccaccaccgccgcctgtcccagttttgcctacgaaccctctaaagatagtaaagtggtggaactcctccgcaatgccggcgccattattgtgggtaaaaccaatctcgatcaatttgccaccggcctcgtgggcacccgcagtccctatggcaaaaccccctgtgccttttccaaagaacacgtgagcggcggtagctctgccggctccgccagcgtggtggcccgcggtattgtgcccattgccctcggcaccgataccgccggctctggtcgggtgcccgccgccctcaataatctcattggcctcaaacccaccaagggtgtgtttagttgtcaaggcgtggtgcccgcctgtaaatctctcgattgtgtgagtatttttgccctcaatctcagcgatgccgaacgctgttttcggattatgtgtcaacccgatcccgataatgatgaatattcccgcccctacgtgagcaatcccctcaaaaaatttagttccaatgtgaccattgccattcccaaaaatattccctggtatggtgaaaccaaaaatcccgtgctcttttccaatgccgtggaaaatctcagccggaccggcgccaatgtgattgaaattgattttgaacccctcctcgaactcgcccgctgtctctatgaaggcacctgggtggccgaacgctatcaagccattcaatcttttctcgatagtaaaccccccaaagaatctctcgatcccaccgtgattagtatcatcgaaggtgccaaaaaatactccgccgtggattgttttagctttgaatacaaacgccaaggcattctccaaaaagtgcgccggctcctcgaatccgtggatgtgctctgtgtgcccacctgtcccctcaatcccaccatgcaacaagtggccgatgaacccgtgctcgtgaatagccgccaaggcacctggaccaattttgtgaatctcgccgatctcgccgccctcgccgtgcccgccggttttcgggatgatggcctccccaatggtattaccctcattggcaaaaaatttaccgattatgccctcctcgaactcgccaaccgctactttcaaaacatctttcccaacggctcccggacctatggcacctttaccagctctagtgtgaaacccgccaatgatcaactcgtgggccccgattacgatccctctaccagtattaaactcgccgtggtgggcgcccacctcaaaggcctccccctccattggcaactcgaaaaagtgaatgccacctatctctgtaccaccaaaaccagcaaagcctaccaactctttgccctccccaaaaatggtcccgtgctcaaacccggcctccgtcgtgtgcaagattccaatggtagccaaattgaactcgaagtgtattccgtgcccaaagaactctttggcgcctttattagcatggtgcccgaacccctcggcattggttctgtggaactcgaaagtggcgaatggatcaaatcttttatttgtgaagaaagtggctacaaagccaaaggcaccgtggatattaccaaatatggcggttttcgggcctactttgaaatgctctaa

SEQ ID NO: 77 - DNA, 1092 bp, Artificial Sequence
  atzD Pseudomonas sp. ADP
atgtatcacatcgacgttttccgaatcccttgccacagccctggtgatacatcgggtctcgaggatttgattgaaacaggccgcgttgcccccgccgacatcgtcgcggtaatgggcaagaccgagggcaatggctgcgtcaacgattacacgcgtgaatacgccaccgccatgcttgctgcgtgccttgggcgtcatttgcaactcccaccccatgaggtggaaaagcgggtcgcgtttgtgatgtcaggtgggacggaaggcgtgctgtccccccaccacacggtattcgcaagacgtccggcaatcgacgcgcatcgtcccgctggcaaacgtctcacgcttggaatcgccttcacgcgtgattttctgccggaggaaattggccgccacgctcagataacggagacagccggcgccgtcaaacgcgcaatgcgagatgccgggatcgcttcgattgacgatctgcattttgtgcaggtgaagtgtccgctgctgacaccagcaaagatcgcctcggcgcgatcacgcggatgcgctccagtcacgacggatacgtatgaatcgatgggctattcgcgcggcgcttcggccctgggcatcgctctcgctacagaagaggtgccctcctcgatgctcgtagacgaatcagtgctgaatgactggagtctctcatcgtcactggcgtcggcgtctgcaggcatcgaactggagcacaacgtggtgatcgctattggcatgagcgagcaggccaccagtgaactggtcattgcccacggcgtgatgagcgacgcgatcgacgcggcctcggtgcggcgaacgattgaatcgctgggcatacgtagcgatgacgagatggatcgcatcgtcaacgtattcgccaaagcggaggcgagcccggacggggttgtacgaggtatgcggcacacgatgctaagtgactccgacattaattcgacccgccatgcgcgggcggtcaccggcgcggccattgcctcggtagttgggcatggcatggtgtatgtgtccggtggcgccgagcatcagggacctgccggcggcggcccttttgcagtcattgcccgcgcttaa

SEQ ID NO: 78 - DNA, 1092 bp, Artificial Sequence
  atzD Pseudomonas sp. ADP
atgtatcacattgatgtgtttcgcattccctgtcatagccccggtgatacctctggcctcgaagatttgattgaaaccggtcgtgtggcccccgccgatattgtggccgtgatgggtaaaaccgagggtaatggctgtgtgaatgattatacccgcgaatacgccaccgccatgctcgccgcctgtctcggccgccacctccaactccccccccatgaagtggaaaaacgggtggcctttgtgatgagtggtggcaccgaaggtgtgctctccccccaccataccgtgtttgcccgtcgccccgccattgatgcccaccgtcccgccggtaaacgtctcaccctcggcattgcctttacccgcgattttctccccgaagaaattggtcggcatgcccaaattaccgaaaccgccggcgccgtgaaacgtgccatgcgtgatgccggtattgccagtattgatgatctccactttgtgcaagtgaaatgtcccctcctcacccccgccaaaattgcctccgcccgcagccggggctgtgcccccgtgaccaccgatacctatgaatctatgggttacagtcggggcgcctccgccctcggtattgccctcgccaccgaagaagtgcccagttccatgctcgtggatgaaagtgtgctcaatgattggtccctcagctctagtctcgcctccgccagcgccggcattgaactcgaacacaatgtggtgattgccattggtatgagtgaacaagccacctccgaactcgtgattgcccacggcgtgatgagcgatgccattgatgccgcctctgtgcgccggaccattgaaagcctcggtattcgctctgatgatgaaatggatcggattgtgaatgtgtttgccaaagccgaagccagtcccgatggtgtggtgcgtggtatgcgtcacaccatgctctctgatagtgatattaattccacccgtcatgcccgtgccgtgaccggtgccgccattgccagcgtggtgggtcacggcatggtgtacgtgtctggcggtgccgaacatcaaggtcccgccggtggtggtccctttgccgtgattgcccgtgcctaa

SEQ ID NO: 79 - DNA, 1320 bp, Artificial Sequence
  guaD Escherichia coli K-12
atgatgtcaggagaacacacgttaaaagcggtacgaggcagttttattgatgtcacccgtacgatcgataacccggaagagattgcctctgcgctgcggtttattgaggatggtttattactcattaaacagggaaaagtggaatggtttggcgaatgggaaaacggaaagcatcaaattcctgacaccattcgcgtgcgcgactatcgcggcaaactgatagtaccgggctttgtcgatacacatatccattatccgcaaagtgaaatggtgggggcctatggtgagcaattgctggagtggttgaataaacacaccttccctactgaacgtcgttatgaggatttagagtacgcccgcgaaatgtcggcgttcttcatcaagcagcttttacgtaacggaaccaccacggcgctggtgtttggcactgttcatccgcaatctgttgatgcgctgtttgaagccgccagtcatatcaatatgcgtatgattgccggtaaggtgatgatggaccgcaacgcaccggattatctgctcgacactgccgaaagcagctatcaccaaagcaaagaactgatcgaacgctggcacaaaaatggtcgtctgctatatgcgattacgccacgcttcgccccgacctcatctcctgaacagatggcgatggcgcaacgcctgaaagaagaatatccggatacgtgggtacatacccatctctgtgaaaacaaagatgaaattgcctgggtgaaatcgctttatcctgaccatgatggttatctggatgtttaccatcagtacggcctgaccggtaaaaactgtgtctttgctcactgcgtccatctcgaagaaaaagagtgggatcgtctcagcgaaaccaaatccagcattgctttctgtccgacctccaacctttacctcggcagcggcttattcaacttgaaaaaagcatggcagaagaaagttaaagtgggcatgggaacggatatcggtgccggaaccactttcaacatgctgcaaacgctgaacgaagcctacaaagtattgcaattacaaggctatcgcctctcggcatatgaagcgttttacctggccacgctcggcggagcgaaatctctgggccttgacgatttgattggcaactttttacctggcaaagaggctgatttcgtggtgatggaacccaccgccactccgctacagcagctgcgctatgacaactctgtttctttagtcgacaaattgttcgtgatgatgacgttgggcgatgaccgttcgatctaccgcacctacgttgatggtcgtctggtgtacgaacgcaactaa

SEQ ID NO: 80 - DNA, 1343 bp, Artificial Sequence
  guaD Escherichia coli K-12
atgatgagcggcgaacacaccctcaaagccgtgcgcggttcttttattgatgtgacccggaccattgataatcccgaagaaattgccagcgccctccgctttattgaagatggcctcctcctcattaaacagggtaaagtggaatggtttggcgaatgggaaaatggtaaacaccaaattcccgataccattcgcgtgcgggattatcggggcaaactcattgtgcccggttttgtggatacccacattcattatccccaaagtgaaatggtgggcgcctacggtgaacaactcctcgaatggctcaataaacatacctttcccaccgaacgccggtatgaagatttggaatacgcccgcgaaatgtccgcctttttcattaaacaactcctccggaatggcaccaccaccgccctcgtgtttggcaccgtgcatccccaaagtgtggatgccctctttgaagccgcctcccatattaatatgcgcatgattgccggcaaagtgatgatggatcggaatgcccccgattatctcctcgataccgccgaaagttcctaccaccaatctaaagaactcattgaacgctggcataaaaatggtcggctcctctatgccattaccccccgttttgcccccaccagctctcccgaacaaatggcgatggcccaacggctcaaagaagaataccccgatacctgggtgcatacccatctctgtgaaaataaagatgaaattgcctgggtgaaaagcctctatcccgatcacgatggctacctcgatgtgtatcatcaatacggcctcaccggtaaaaattgtgtgtttgcccactgtgtgcatctcgaagaaaaagaatgggatcgcctctctgaaaccaaaagttccattgccttttgtcccaccagcaatctctatctcggctctggcctctttaatctcaaaaaagcctggcagaaaaaagtgaaagtgggcatgggcaccgatattggcgccggcaccacctttaatatgctccaaaccctcaatgaagcctataaagtgctccaactccaaggctacagcctctctgcctatgaagccttttacctcgccaccctcggcggtgccaaaagtctcggcctcgatgatctcattggcaattttctccccggtaaagaagccgattttgtggtgatggaacccaccgccacccccctccaacaactccgttatgataatagtgtgtccctcgtggagggtttgcatggtctgaatctgataaactctttgtgatgatgaccctcggcgatgatcgctccatttatcggacctacgtggatggtcgcctcgtgtacgaacggaattaa

SEQ ID NO: 81 - DNA, 1320 bp, Artificial Sequence
  guaD R352S Escherichia coli K-12
atgatgtcaggagaacacacgttaaaagcggtacgaggcagttttattgatgtcacccgtacgatcgataacccggaagagattgcctctgcgctgcggtttattgaggatggtttattactcattaaacagggaaaagtggaatggtttggcgaatgggaaaacggaaagcatcaaattcctgacaccattcgcgtgcgcgactatcgcggcaaactgatagtaccgggctttgtcgatacacatatccattatccgcaaagtgaaatggtgggggcctatggtgagcaattgctggagtggttgaataaacacaccttccctactgaacgtcgttatgaggatttagagtacgcccgcgaaatgtcggcgttcttcatcaagcagcttttacgtaacggaaccaccacggcgctggtgtttggcactgttcatccgcaatctgttgatgcgctgtttgaagccgccagtcatatcaatatgcgtatgattgccggtaaggtgatgatggaccgcaacgcaccggattatctgctcgacactgccgaaagcagctatcaccaaagcaaagaactgatcgaacgctggcacaaaaatggtcgtctgctatatgcgattacgccacgcttcgccccgacctcatctcctgaacagatggcgatggcgcaacgcctgaaagaagaatatccggatacgtgggtacatacccatctctgtgaaaacaaagatgaaattgcctgggtgaaatcgctttatcctgaccatgatggttatctggatgtttaccatcagtacggcctgaccggtaaaaactgtgtctttgctcactgcgtccatctcgaagaaaaagagtgggatcgtctcagcgaaaccaaatccagcattgctttctgtccgacctccaacctttacctcggcagcggcttattcaacttgaaaaaagcatggcagaagaaagttaaagtgggcatgggaacggatatcggtgccggaaccactttcaacatgctgcaaacgctgaacgaagcctacaaagtattgcaattacaaggctatagcctctcggcatatgaagcgttttacctggccacgctcggcggagcgaaatctctgggccttgacgatttgattggcaactttttacctggcaaagaggctgatttcgtggtgatggaacccaccgccactccgctacagcagctgcgctatgacaactctgtttctttagtcgacaaattgttcgtgatgatgacgttgggcgatgaccgttcgatctaccgcacctacgttgatggtcgtctggtgtacgaacgcaactaa

SEQ ID NO: 82 - DNA, 89 bp, Artificial Sequence
  Pc223 promoter
ttaacaaaaaagcaggaataaaattaacatgatgtaacagacataagtcccatcaccgttgtataatgttaactgtgggattgcaaaag

SEQ ID NO: 83 - DNA, 7983 bp, Artificial Sequence
  Mel1
  promoter (1)..(89): Pc223 promoter
  gene (104)..(1195): atzd
  gene (1217)..(1942): trzE
  gene (1963)..(3831): DUR1,2
  gene (3853)..(5091): trzC
  gene (5113)..(6432): guaD
  gene (6454)..(7878): triA
  terminator (7929)..(7957): RRNB terminator
ttaacaaaaaagcaggaataaaattaacatgatgtaacagacataagtcccatcaccgttgtataatgttaactgtgggattgcaaaagaggagattaattcaatgtatcacattgatgtgtttcgcattccctgtcatagccccggtgatacctctggcctcgaagatttgattgaaaccggtcgtgtggcccccgccgatattgtggccgtgatgggtaaaaccgagggtaatggctgtgtgaatgattatacccgcgaatacgccaccgccatgctcgccgcctgtctcggccgccacctccaactccccccccatgaagtggaaaaacgggtggcctttgtgatgagtggtggcaccgaaggtgtgctctccccccaccataccgtgtttgcccgtcgccccgccattgatgcccaccgtcccgccggtaaacgtctcaccctcggcattgcctttacccgcgattttctccccgaagaaattggtcggcatgcccaaattaccgaaaccgccggcgccgtgaaacgtgccatgcgtgatgccggtattgccagtattgatgatctccactttgtgcaagtgaaatgtcccctcctcacccccgccaaaattgcctccgcccgcagccggggctgtgcccccgtgaccaccgatacctatgaatctatgggttacagtcggggcgcctccgccctcggtattgccctcgccaccgaagaagtgcccagttccatgctcgtggatgaaagtgtgctcaatgattggtccctcagctctagtctcgcctccgccagcgccggcattgaactcgaacacaatgtggtgattgccattggtatgagtgaacaagccacctccgaactcgtgattgcccacggcgtgatgagcgatgccattgatgccgcctctgtgcgccggaccattgaaagcctcggtattcgctctgatgatgaaatggatcggattgtgaatgtgtttgccaaagccgaagccagtcccgatggtgtggtgcgtggtatgcgtcacaccatgctctctgatagtgatattaattccacccgtcatgcccgtgccgtgaccggtgccgccattgccagcgtggtgggtcacggcatggtgtacgtgtctggcggtgccgaacatcaaggtcccgccggtggtggtccctttgccgtgattgcccgtgcctaagtcaagtaggagattaattccatgatttatagtaccgtgaatgccaatccctatgcctggccctacgatggctccattgatcccgcccacaccgccctcatcctcatcgattggcaaatcgatttttgtggtcccggcggttatgtggatagtatgggctacgatctcagcctcacccgctctggcctcgaacccaccgcccgcgtgctcgccgccgcccgcgataccggcatgaccgtgattcacacccgcgaaggtcatcgtcccgatctcgccgatctcccccccaataaacgttggcggagtgcctccgccggcgccgaaattggcagtgtgggtccctgtggccgcattctcgtgcgtggtgaacccggttgggaaattgtgcccgaagtggccccccgcgaaggtgaacccattattgataaacccggtaaaggtgccttttatgccaccgatctcgatctcctcctccgcacccgcggcattacccacctcattctcaccggcattaccaccgatgtgtgtgtgcataccaccatgcgcgaagccaatgatcggggctacgaatgtctcattctctccgattgtaccggtgccaccgatcggaaacaccatgaagccgccctcagcatggtgaccatgcaaggcggtgtgtttggtgccaccgcccattctgatgatctcctcgccgccctcggcaccaccgtgcccgccgccgccggcccccgtgcccgcaccgaataatctaataggagacaaatactatgaccgtgagttccgataccaccgccgaaatttctctcggctggagtatccaagattggatcgattttcacaaaagctctagttcccaagcctccctccgcctcctcgaatccctcctcgatagccaaaatgtggcccccgtggataatgcctggatttccctcattagcaaggaaaacctcctccatcaatttcaaattctcaagtctcgcgaaaacaaggaaaccctccccctctatggtgtgcccattgccgtgaaagataatattgatgtgcgtggcctccccaccaccgccgcctgtcccagttttgcctacgaaccctctaaagatagtaaagtggtggaactcctccgcaatgccggcgccattattgtgggtaaaaccaatctcgatcaatttgccaccggcctcgtgggcacccgcagtccctatggcaaaaccccctgtgccttttccaaagaacacgtgagcggcggtagctctgccggctccgccagcgtggtggcccgcggtattgtgcccattgccctcggcaccgataccgccggctctggtcgggtgcccgccgccctcaataatctcattggcctcaaacccaccaagggtgtgtttagttgtcaaggcgtggtgcccgcctgtaaatctctcgattgtgtgagtatttttgccctcaatctcagcgatgccgaacgctgttttcggattatgtgtcaacccgatcccgataatgatgaatattcccgcccctacgtgagcaatcccctcaaaaaatttagttccaatgtgaccattgccattcccaaaaatattccctggtatggtgaaaccaaaaatcccgtgctcttttccaatgccgtggaaaatctcagccggaccggcgccaatgtgattgaaattgattttgaacccctcctcgaactcgcccgctgtctctatgaaggcacctgggtggccgaacgctatcaagccattcaatcttttctcgatagtaaaccccccaaagaatctctcgatcccaccgtgattagtatcatcgaaggtgccaaaaaatactccgccgtggattgttttagctttgaatacaaacgccaaggcattctccaaaaagtgcgccggctcctcgaatccgtggatgtgctctgtgtgcccacctgtcccctcaatcccaccatgcaacaagtggccgatgaacccgtgctcgtgaatagccgccaaggcacctggaccaattttgtgaatctcgccgatctcgccgccctcgccgtgcccgccggttttcgggatgatggcctccccaatggtattaccctcattggcaaaaaatttaccgattatgccctcctcgaactcgccaaccgctactttcaaaacatctttcccaacggctcccggacctatggcacctttaccagctctagtgtgaaacccgccaatgatcaactcgtgggccccgattacgatccctctaccagtattaaactcgccgtggtgggcgcccacctcaaaggcctccccctccattggcaactcgaaaaagtgaatgccacctatctctgtaccaccaaaaccagcaaagcctaccaactctttgccctccccaaaaatggtcccgtgctcaaacccggcctccgtcgtgtgcaagattccaatggtagccaaattgaactcgaagtgtattccgtgcccaaagaactctttggcgcctttattagcatggtgcccgaacccctcggcattggttctgtggaactcgaaagtggcgaatggatcaaatcttttatttgtgaagaaagtggctacaaagccaaaggcaccgtggatattaccaaatatggcggttttcgggcctactttgaaatgctctaaattgattaggagatatacaccatgagcatggaaacccactcctatgttgatgttgcgatccgtaacgcccgtctcgccgataccgaaggcattgttgatattctgattcatgatggtcggattgccagcattgtgaaatccaccaaaaccaaaggttccgtggaaattgatgcccatgaaggtctcgtgaccagtggcctcgtggaaccccacattcatctcgataaagccctcaccgccgatcgcgtgcccgccggtagtattggcgatctccggacccgccggggtctcgaaatggccattcgcgccacccgcgatattaaacgcacctttaccgtggaagatgtgcgggaacgcgccattcgggccgccctcatggccagtcgcgccggcaccaccgccctccgcacccatgtggatgtggaccccattgtgggtctcgccggtattcggggcgtgctcgaagcccgcgaagtgtgtgccggtctcattgatattcaaattgtggcctttccccaagaaggcctcttttgtagtgccggtgccgtggatctcatgcgggaagccattaaactcggtgccgatgccgtgggcggtgcccccgccctcgatgatcgcccccaagatcatgtgcgcgccgtgtttgatctcgccgccgaatttggtctccccgtggatatgcacgtggatgaaagtgatcgccgggaagattttaccctcccctttgtgattgaagccgcccgcgaacggcgcgtgcccaatgtgaccgtggcccacatttcctctctcagtgtgcaaaccgatgatgtggcccgctccaccattgccgccctcgccgatgccgatgtgaatgtggtggtgaatcccattattgtgaaaattacccgcctctctgaactcctcgatgccggtgtgtctgtgatgtttggctccgataatctccgcgatcccttttatcccctcggtgccgccaatcccctcggtagtgccatttttgcctgtcaaattgccgccctcggcaccccccaagacctccgccgggtgtttgatgccgtgaccattaatgccgcccgcatgctcggttttcccagtctcctcggtgtggtggaaggtgccgtggccgatctcgccgtgtttcccagtgccacccccgaagaagtggtgctcgatcaacaaagtcccctctttgtgctcaaaggtggtcgggtcgttgcgatgcggttagcggcgggtagcacctcctttcgtgattattcgtaatatacttaggagattcatacgatgatgagcggcgaacacaccctcaaagccgtgcgcggttcttttattgatgtgacccggaccattgataatcccgaagaaattgccagcgccctccgctttattgaagatggcctcctcctcattaaacagggtaaagtggaatggtttggcgaatgggaaaatggtaaacaccaaattcccgataccattcgcgtgcgggattatcggggcaaactcattgtgcccggttttgtggatacccacattcattatccccaaagtgaaatggtgggcgcctacggtgaacaactcctcgaatggctcaataaacatacctttcccaccgaacgccggtatgaagatttggaatacgcccgcgaaatgtccgcctttttcattaaacaactcctccggaatggcaccaccaccgccctcgtgtttggcaccgtgcatccccaaagtgtggatgccctctttgaagccgcctcccatattaatatgcgcatgattgccggcaaagtgatgatggatcggaatgcccccgattatctcctcgataccgccgaaagttcctaccaccaatctaaagaactcattgaacgctggcataaaaatggtcggctcctctatgccattaccccccgttttgcccccaccagctctcccgaacaaatggcgatggcccaacggctcaaagaagaataccccgatacctgggtgcatacccatctctgtgaaaataaagatgaaattgcctgggtgaaaagcctctatcccgatcacgatggctacctcgatgtgtatcatcaatacggcctcaccggtaaaaattgtgtgtttgcccactgtgtgcatctcgaagaaaaagaatgggatcgcctctctgaaaccaaaagttccattgccttttgtcccaccagcaatctctatctcggctctggcctctttaatctcaaaaaagcctggcagaaaaaagtgaaagtgggcatgggcaccgatattggcgccggcaccacctttaatatgctccaaaccctcaatgaagcctataaagtgctccaactccaaggctacagcctctctgcctatgaagccttttacctcgccaccctcggcggtgccaaaagtctcggcctcgatgatctcattggcaattttctccccggtaaagaagccgattttgtggtgatggaacccaccgccacccccctccaacaactccgttatgataatagtgtgtccctcgtggataaactctttgtgatgatgaccctcggcgatgatcgctccatttatcggacctacgtggatggtcgcctcgtgtacgaacggaattaagctagttaggagattcagaccatgcaaaccctcagcattcaacatggcaccctcgtgacgatggatcaatatcgccgggtgctcggcgatagctgggtgcatgtgcaagatggccgcattgtggccctcggtgtgcatgccgaatctgtgcccccccccgccgatcgtgtgattgatgcccgcggtaaagtggtgctccccggttttattaatgcccatacccacgtgaatcaaattctcctccgtggtggtccctctcacggtcgccaactctatgattggctctttaatgtgctctaccccggccaaaaagccatgcgccccgaagatgtggccgtggccgtgcggctctattgtgccgaagccgtgcgcagtggtattaccaccattaatgataatgccgattccgccatttaccccggcaatattgaagccgcgatggccgtgtatggcgaagtgggtgtgcgggtggtgtacgcccgcatgtttttcgatcgcatggatggccggattcaaggttatgtggatgccctcaaagcccggagcccccaagtggaactctgttctattatggaagaaaccgccgtggccaaagatcggattaccgccctcagcgatcaatatcacggcaccgccggtggccgcattagtgtgtggcccgcccccgccattacccccgccgtgaccgtggagggtatgcgttgggcccaagcctttgcccgcgatcgggccgtgatgtggaccctccacatggccgaaagcgatcatgatgaacggctccactggatgtctcccgccgaatatatggaatgttacggcctcctcgatgaacgcctccaagtggcccactgtgtgtattttgatcgcaaagatgtgcggctcctccatcgccacaatgtgaaagtggccagtcaagtggtgtccaatgcctacctcggcagtggtgtggcccccgtgcccgaaatggtggaacgtggcatggccgtgggcattggcaccgatgatggtaattgtaatgattccgtgaatatgattggcgatatgaaatttatggcccatattcaccgggccgtgcatcgcgatgccgatgtgctcacccccgaaaaaattctcgaaatggccaccattgatggcgcccgcagtctcggtatggatcatgaaattggctccattgaaaccggtaaacgggccgatctcattctcctcgatctccgccacccccaaaccaccccccaccatcacctcgccgccaccattgtgtttcaagcctacggtaatgaagtggataccgtgctcattgatggcaatgtggtgatggaaaatcgccggctcagttttctcccccccgaacgggaactcgcctttctcgaagaagcccaaagtcgcgccaccgccattctccaacgcgccaatatggtggccaatcccgcctgacgcagcctctaaaagggtgggcgcgccgacccagctttcttgtacaaactcggccctgcaggagaaggccatcctgacggatggcctttttgaagcttgaaagcaaatgacgagcga

SEQ ID NO: 84 - DNA, 7968 bp, Artificial Sequence
  Mel4
  promoter (1)..(89): Pc223 promoter
  gene (104)..(1195): atzD
  gene (1217)..(1942): trzE
  gene (1963)..(3831): DUR1,2
  gene (3853)..(5091): trzC
  gene (5113)..(6432): guaD
  gene (6454)..(7878): triA
  terminator (7929)..(7957): RRNB terminator
ttaacaaaaaagcaggaataaaattaacatgatgtaacagacataagtcccatcaccgttgtataatgttaactgtgggattgcaaaagaggagattaattcaatgtatcacattgatgtgtttcgcattccctgtcatagccccggtgatacctctggcctcgaagatttgattgaaaccggtcgtgtggcccccgccgatattgtggccgtgatgggtaaaaccgagggtaatggctgtgtgaatgattatacccgcgaatacgccaccgccatgctcgccgcctgtctcggccgccacctccaactccccccccatgaagtggaaaaacgggtggcctttgtgatgagtggtggcaccgaaggtgtgctctccccccaccataccgtgtttgcccgtcgccccgccattgatgcccaccgtcccgccggtaaacgtctcaccctcggcattgcctttacccgcgattttctccccgaagaaattggtcggcatgcccaaattaccgaaaccgccggcgccgtgaaacgtgccatgcgtgatgccggtattgccagtattgatgatctccactttgtgcaagtgaaatgtcccctcctcacccccgccaaaattgcctccgcccgcagccggggctgtgcccccgtgaccaccgatacctatgaatctatgggttacagtcggggcgcctccgccctcggtattgccctcgccaccgaagaagtgcccagttccatgctcgtggatgaaagtgtgctcaatgattggtccctcagctctagtctcgcctccgccagcgccggcattgaactcgaacacaatgtggtgattgccattggtatgagtgaacaagccacctccgaactcgtgattgcccacggcgtgatgagcgatgccattgatgccgcctctgtgcgccggaccattgaaagcctcggtattcgctctgatgatgaaatggatcggattgtgaatgtgtttgccaaagccgaagccagtcccgatggtgtggtgcgtggtatgcgtcacaccatgctctctgatagtgatattaattccacccgtcatgcccgtgccgtgaccggtgccgccattgccagcgtggtgggtcacggcatggtgtacgtgtctggcggtgccgaacatcaaggtcccgccggtggtggtccctttgccgtgattgcccgtgcctaagtcaagtaggagattaattccatgatttatagtaccgtgaatgccaatccctatgcctggccctacgatggctccattgatcccgcccacaccgccctcatcctcatcgattggcaaatcgatttttgtggtcccggcggttatgtggatagtatgggctacgatctcagcctcacccgctctggcctcgaacccaccgcccgcgtgctcgccgccgcccgcgataccggcatgaccgtgattcacacccgcgaaggtcatcgtcccgatctcgccgatctcccccccaataaacgttggcggagtgcctccgccggcgccgaaattggcagtgtgggtccctgtggccgcattctcgtgcgtggtgaacccggttgggaaattgtgcccgaagtggccccccgcgaaggtgaacccattattgataaacccggtaaaggtgccttttatgccaccgatctcgatctcctcctccgcacccgcggcattacccacctcattctcaccggcattaccaccgatgtgtgtgtgcataccaccatgcgcgaagccaatgatcggggctacgaatgtctcattctctccgattgtaccggtgccaccgatcggaaacaccatgaagccgccctcagcatggtgaccatgcaaggcggtgtgtttggtgccaccgcccattctgatgatctcctcgccgccctcggcaccaccgtgcccgccgccgccggcccccgtgcccgcaccgaataatctaataggagacaaatactatgaccgtgagttccgataccaccgccgaaatttctctcggctggagtatccaagattggatcgattttcacaaaagctctagttcccaagcctccctccgcctcctcgaatccctcctcgatagccaaaatgtggcccccgtggataatgcctggatttccctcattagcaaggaaaacctcctccatcaatttcaaattctcaagtctcgcgaaaacaaggaaaccctccccctctatggtgtgcccattgccgtgaaagataatattgatgtgcgtggcctccccaccaccgccgcctgtcccagttttgcctacgaaccctctaaagatagtaaagtggtggaactcctccgcaatgccggcgccattattgtgggtaaaaccaatctcgatcaatttgccaccggcctcgtgggcacccgcagtccctatggcaaaaccccctgtgccttttccaaagaacacgtgagcggcggtagctctgccggctccgccagcgtggtggcccgcggtattgtgcccattgccctcggcaccgataccgccggctctggtcgggtgcccgccgccctcaataatctcattggcctcaaacccaccaagggtgtgtttagttgtcaaggcgtggtgcccgcctgtaaatctctcgattgtgtgagtatttttgccctcaatctcagcgatgccgaacgctgttttcggattatgtgtcaacccgatcccgataatgatgaatattcccgcccctacgtgagcaatcccctcaaaaaatttagttccaatgtgaccattgccattcccaaaaatattccctggtatggtgaaaccaaaaatcccgtgctcttttccaatgccgtggaaaatctcagccggaccggcgccaatgtgattgaaattgattttgaacccctcctcgaactcgcccgctgtctctatgaaggcacctgggtggccgaacgctatcaagccattcaatcttttctcgatagtaaaccccccaaagaatctctcgatcccaccgtgattagtatcatcgaaggtgccaaaaaatactccgccgtggattgttttagctttgaatacaaacgccaaggcattctccaaaaagtgcgccggctcctcgaatccgtggatgtgctctgtgtgcccacctgtcccctcaatcccaccatgcaacaagtggccgatgaacccgtgctcgtgaatagccgccaaggcacctggaccaattttgtgaatctcgccgatctcgccgccctcgccgtgcccgccggttttcgggatgatggcctccccaatggtattaccctcattggcaaaaaatttaccgattatgccctcctcgaactcgccaaccgctactttcaaaacatctttcccaacggctcccggacctatggcacctttaccagctctagtgtgaaacccgccaatgatcaactcgtgggccccgattacgatccctctaccagtattaaactcgccgtggtgggcgcccacctcaaaggcctccccctccattggcaactcgaaaaagtgaatgccacctatctctgtaccaccaaaaccagcaaagcctaccaactctttgccctccccaaaaatggtcccgtgctcaaacccggcctccgtcgtgtgcaagattccaatggtagccaaattgaactcgaagtgtattccgtgcccaaagaactctttggcgcctttattagcatggtgcccgaacccctcggcattggttctgtggaactcgaaagtggcgaatggatcaaatcttttatttgtgaagaaagtggctacaaagccaaaggcaccgtggatattaccaaatatggcggttttcgggcctactttgaaatgctctaaattgattaggagatatacaccatgagcatggaaacccactcctatgttgatgttgcgatccgtaacgcccgtctcgccgataccgaaggcattgttgatattctgattcatgatggtcggattgccagcattgtgaaatccaccaaaaccaaaggttccgtggaaattgatgcccatgaaggtctcgtgaccagtggcctcgtggaaccccacattcatctcgataaagccctcaccgccgatcgcgtgcccgccggtagtattggcgatctccggacccgccggggtctcgaaatggccattcgcgccacccgcgatattaaacgcacctttaccgtggaagatgtgcgggaacgcgccattcgggccgccctcatggccagtcgcgccggcaccaccgccctccgcacccatgtggatgtggaccccattgtgggtctcgccggtattcggggcgtgctcgaagcccgcgaagtgtgtgccggtctcattgatattcaaattgtggcctttccccaagaaggcctcttttgtagtgccggtgccgtggatctcatgcgggaagccattaaactcggtgccgatgccgtgggcggtgcccccgccctcgatgatcgcccccaagatcatgtgcgcgccgtgtttgatctcgccgccgaatttggtctccccgtggatatgcacgtggatgaaagtgatcgccgggaagattttaccctcccctttgtgattgaagccgcccgcgaacggcgcgtgcccaatgtgaccgtggcccacatttcctctctcagtgtgcaaaccgatgatgtggcccgctccaccattgccgccctcgccgatgccgatgtgaatgtggtggtgaatcccattattgtgaaaattacccgcctctctgaactcctcgatgccggtgtgtctgtgatgtttggctccgataatctccgcgatcccttttatcccctcggtgccgccaatcccctcggtagtgccatttttgcctgtcaaattgccgccctcggcaccccccaagacctccgccgggtgtttgatgccgtgaccattaatgccgcccgcatgctcggttttcccagtctcctcggtgtggtggaaggtgccgtggccgatctcgccgtgtttcccagtgccacccccgaagaagtggtgctcgatcaacaaagtcccctctttgtgctcaaaggtggtcgggtcgttgcgatgcggttagcggcgggtagcacctcctttcgtgattattcgtaatatacttaggagattcatacgatgatgagcggcgaacacaccctcaaagccgtgcgcggttcttttattgatgtgacccggaccattgataatcccgaagaaattgccagcgccctccgctttattgaagatggcctcctcctcattaaacagggtaaagtggaatggtttggcgaatgggaaaatggtaaacaccaaattcccgataccattcgcgtgcgggattatcggggcaaactcattgtgcccggttttgtggatacccacattcattatccccaaagtgaaatggtgggcgcctacggtgaacaactcctcgaatggctcaataaacatacctttcccaccgaacgccggtatgaagatttggaatacgcccgcgaaatgtccgcctttttcattaaacaactcctccggaatggcaccaccaccgccctcgtgtttggcaccgtgcatccccaaagtgtggatgccctctttgaagccgcctcccatattaatatgcgcatgattgccggcaaagtgatgatggatcggaatgcccccgattatctcctcgataccgccgaaagttcctaccaccaatctaaagaactcattgaacgctggcataaaaatggtcggctcctctatgccattaccccccgttttgcccccaccagctctcccgaacaaatggcgatggcccaacggctcaaagaagaataccccgatacctgggtgcatacccatctctgtgaaaataaagatgaaattgcctgggtgaaaagcctctatcccgatcacgatggctacctcgatgtgtatcatcaatacggcctcaccggtaaaaattgtgtgtttgcccactgtgtgcatctcgaagaaaaagaatgggatcgcctctctgaaaccaaaagttccattgccttttgtcccaccagcaatctctatctcggctctggcctctttaatctcaaaaaagcctggcagaaaaaagtgaaagtgggcatgggcaccgatattggcgccggcaccacctttaatatgctccaaaccctcaatgaagcctataaagtgctccaactccaaggctacagcctctctgcctatgaagccttttacctcgccaccctcggcggtgccaaaagtctcggcctcgatgatctcattggcaattttctccccggtaaagaagccgattttgtggtgatggaacccaccgccacccccctccaacaactccgttatgataatagtgtgtccctcgtggataaactctttgtgatgatgaccctcggcgatgatcgctccatttatcggacctacgtggatggtcgcctcgtgtacgaacggaattaagctagttaggagattcagaccatgcaaaccctcagcattcaacatggcaccctcgtgacgatggatcaatatcgccgggtgctcggcgatagctgggtgcatgtgcaagatggccgcattgtggccctcggtgtgcatgccgaatctgtgcccccccccgccgatcgtgtgattgatgcccgcggtaaagtggtgctccccggttttattaatgcccatacccacgtgaatcaaattctcctccgtggtggtccctctcacggtcgccaactctatgattggttctttaatgtgctctaccccggccaaaaagccatgcgccccgaagatgtggccgtggccgtgcggctctattgtgccgaagccgtgcgcagtggtattaccaccattaatgataatgccgattccgccatttaccccggcaatattgaagccgcgatggccgtgtatggcgaagtgggtgtgcgggtggtgtacgcccgcatgtttttcgatcgcatggatggccggattcaaggttatgtggatgccctcaaagcccggagcccccaagtggaactctgttctattatggaagaaaccgccgtggccaaagatcggattaccgccctcagcgatcaatatcacggcaccgccggtggccgcattagtgtgtggcccgcccccgccattacccccgccgtgaccgtggagggtatgcgttgggcccaagcctttgcccgcgatcgggccgtgatgtggaccctccacatggccgaaagcgatcatgatgaacggctccactggatgtctcccgccgaatatatggaatgttacggcctcctcgatgaacgcctccaagtggcccactgtgtgtattttgatcgcaaagatgtgcggctcctccatcgccacaatgtgaaagtggccagtcaagtggtgtccaatgcctacctcggcagtggtgtggcccccgtgcccgaaatggtggaacgtggcatggccgtgggcattggcaccgatgatggtaattgtaatgattccgtgaatatgattggcgatatgaaatttatggcccatattcaccgggccgtgcatcgcgatgccgatgtgctcacccccgaaaaaattctcgaaatggccaccattgatggcgcccgcagtctcggtatggatcatgaaattggctccattgaaaccggtaaacgggccgatctcattctcctcgatctccgccacccccaaaccaccccccaccatcacctcgccgccaccattgtgtttcaagcctacggtaatgaagtggataccgtgctcattgatggcaatgtggtgatggaaaatcgccggctcagttttctcccccccgaacgggaactcgcctttctcgaagaagcccaaagtcgcgccaccgccattctccaacgcgccaatatggtggccaatcccgcctggcgcagcctctaaaagggtgggcgcgccgacccagctttcttgtacaaactcggccctgcaggagaaggccatcctgacggatggcctttttgaagcttgaaa

SEQ ID NO: 85 - DNA, 7968 bp, Artificial Sequence
  Mel5
  promoter (1)..(89): Pc223 promoter
  gene (104)..(1195): atzD
  gene (1217)..(1942): trzE
  gene (1963)..(3831): DUR1,2
  gene (3853)..(5091): trzC
  gene (5113)..(6432): guaD
  gene (6454)..(7878): triA
  terminator (7929)..(7957): RRNB terminator
ttaacaaaaaagcaggaataaaattaacatgatgtaacagacataagtcccatcaccgttgtataatgttaactgtgggattgcaaaagaggagattaattcaatgtatcacattgatgtgtttcgcattccctgtcatagccccggtgatacctctggcctcgaagatttgattgaaaccggtcgtgtggcccccgccgatattgtggccgtgatgggtaaaaccgagggtaatggctgtgtgaatgattatacccgcgaatacgccaccgccatgctcgccgcctgtctcggccgccacctccaactccccccccatgaagtggaaaaacgggtggcctttgtgatgagtggtggcaccgaaggtgtgctctccccccaccataccgtgtttgcccgtcgccccgccattgatgcccaccgtcccgccggtaaacgtctcaccctcggcattgcctttacccgcgattttctccccgaagaaattggtcggcatgcccaaattaccgaaaccgccggcgccgtgaaacgtgccatgcgtgatgccggtattgccagtattgatgatctccactttgtgcaagtgaaatgtcccctcctcacccccgccaaaattgcctccgcccgcagccggggctgtgcccccgtgaccaccgatacctatgaatctatgggttacagtcggggcgcctccgccctcggtattgccctcgccaccgaagaagtgcccagttccatgctcgtggatgaaagtgtgctcaatgattggtccctcagctctagtctcgcctccgccagcgccggcattgaactcgaacacaatgtggtgattgccattggtatgagtgaacaagccacctccgaactcgtgattgcccacggcgtgatgagcgatgccattgatgccgcctctgtgcgccggaccattgaaagcctcggtattcgctctgatgatgaaatggatcggattgtgaatgtgtttgccaaagccgaagccagtcccgatggtgtggtgcgtggtatgcgtcacaccatgctctctgatagtgatattaattccacccgtcatgcccgtgccgtgaccggtgccgccattgccagcgtggtgggtcacggcatggtgtacgtgtctggcggtgccgaacatcaaggtcccgccggtggtggtccctttgccgtgattgcccgtgcctaagtcaagtaggagattaattccatgatttatagtaccgtgaatgccaatccctatgcctggccctacgatggctccattgatcccgcccacaccgccctcatcctcatcgattggcaaatcgatttttgtggtcccggcggttatgtggatagtatgggctacgatctcagcctcacccgctctggcctcgaacccaccgcccgcgtgctcgccgccgcccgcgataccggcatgaccgtgattcacacccgcgaaggtcatcgtcccgatctcgccgatctcccccccaataaacgttggcggagtgcctccgccggcgccgaaattggcagtgtgggtccctgtggccgcattctcgtgcgtggtgaacccggttgggaaattgtgcccgaagtggccccccgcgaaggtgaacccattattgataaacccggtaaaggtgccttttatgccaccgatctcgatctcctcctccgcacccgcggcattacccacctcattctcaccggcattaccaccgatgtgtgtgtgcataccaccatgcgcgaagccaatgatcggggctacgaatgtctcattctctccgattgtaccggtgccaccgatcggaaacaccatgaagccgccctcagcatggtgaccatgcaaggcggtgtgtttggtgccaccgcccattctgatgatctcctcgccgccctcggcaccaccgtgcccgccgccgccggcccccgtgcccgcaccgaataatctaataggagacaaatactatgaccgtgagttccgataccaccgccgaaatttctctcggctggagtatccaagattggatcgattttcacaaaagctctagttcccaagcctccctccgcctcctcgaatccctcctcgatagccaaaatgtggcccccgtggataatgcctggatttccctcattagcaaggaaaacctcctccatcaatttcaaattctcaagtctcgcgaaaacaaggaaaccctccccctctatggtgtgcccattgccgtgaaagataatattgatgtgcgtggcctccccaccaccgccgcctgtcccagttttgcctacgaaccctctaaagatagtaaagtggtggaactcctccgcaatgccggcgccattattgtgggtaaaaccaatctcgatcaatttgccaccggcctcgtgggcacccgcagtccctatggcaaaaccccctgtgccttttccaaagaacacgtgagcggcggtagctctgccggctccgccagcgtggtggcccgcggtattgtgcccattgccctcggcaccgataccgccggctctggtcgggtgcccgccgccctcaataatctcattggcctcaaacccaccaagggtgtgtttagttgtcaaggcgtggtgcccgcctgtaaatctctcgattgtgtgagtatttttgccctcaatctcagcgatgccgaacgctgttttcggattatgtgtcaacccgatcccgataatgatgaatattcccgcccctacgtgagcaatcccctcaaaaaatttagttccaatgtgaccattgccattcccaaaaatattccctggtatggtgaaaccaaaaatcccgtgctcttttccaatgccgtggaaaatctcagccggaccggcgccaatgtgattgaaattgattttgaacccctcctcgaactcgcccgctgtctctatgaaggcacctgggtggccgaacgctatcaagccattcaatcttttctcgatagtaaaccccccaaagaatctctcgatcccaccgtgattagtatcatcgaaggtgccaaaaaatactccgccgtggattgttttagctttgaatacaaacgccaaggcattctccaaaaagtgcgccggctcctcgaatccgtggatgtgctctgtgtgcccacctgtcccctcaatcccaccatgcaacaagtggccgatgaacccgtgctcgtgaatagccgccaaggcacctggaccaattttgtgaatctcgccgatctcgccgccctcgccgtgcccgccggttttcgggatgatggcctccccaatggtattaccctcattggcaaaaaatttaccgattatgccctcctcgaactcgccaaccgctactttcaaaacatctttcccaacggctcccggacctatggcacctttaccagctctagtgtgaaacccgccaatgatcaactcgtgggccccgattacgatccctctaccagtattaaactcgccgtggtgggcgcccacctcaaaggcctccccctccattggcaactcgaaaaagtgaatgccacctatctctgtaccaccaaaaccagcaaagcctaccaactctttgccctccccaaaaatggtcccgtgctcaaacccggcctccgtcgtgtgcaagattccaatggtagccaaattgaactcgaagtgtattccgtgcccaaagaactctttggcgcctttattagcatggtgcccgaacccctcggcattggttctgtggaactcgaaagtggcgaatggatcaaatcttttatttgtgaagaaagtggctacaaagccaaaggcaccgtggatattaccaaatatggcggttttcgggcctactttgaaatgctctaaattgattaggagatatacaccatgagcatggaaacccactcctatgttgatgttgcgatccgtaacgcccgtctcgccgataccgaaggcattgttgatattctgattcatgatggtcggattgccagcattgtgaaatccaccaaaaccaaaggttccgtggaaattgatgcccatgaaggtctcgtgaccagtggcctcgtggaaccccacattcatctcgataaagccctcaccgccgatcgcgtgcccgccggtagtattggcgatctccggacccgccggggtctcgaaatggccattcgcgccacccgcgatattaaacgcacctttaccgtggaagatgtgcgggaacgcgccattcgggccgccctcatggccagtcgcgccggcaccaccgccctccgcacccatgtggatgtggaccccattgtgggtctcgccggtattcggggcgtgctcgaagcccgcgaagtgtgtgccggtctcattgatattcaaattgtggcctttccccaagaaggcctcttttgtagtgccggtgccgtggatctcatgcgggaagccattaaactcggtgccgatgccgtgggcggtgcccccgccctcgatgatcgcccccaagatcatgtgcgcgccgtgtttgatctcgccgccgaatttggtctccccgtggatatgcacgtggatgaaagtgatcgccgggaagattttaccctcccctttgtgattgaagccgcccgcgaacggcgcgtgcccaatgtgaccgtggcccacatttcctctctcagtgtgcaaaccgatgatgtggcccgctccaccattgccgccctcgccgatgccgatgtgaatgtggtggtgaatcccattattgtgaaaattacccgcctctctgaactcctcgatgccggtgtgtctgtgatgtttggctccgataatctccgcgatcccttttatcccctcggtgccgccaatcccctcggtagtgccatttttgcctgtcaaattgccgccctcggcaccccccaagacctccgccgggtgtttgatgccgtgaccattaatgccgcccgcatgctcggttttcccagtctcctcggtgtggtggaaggtgccgtggccgatctcgccgtgtttcccagtgccacccccgaagaagtggtgctcgatcaacaaagtcccctctttgtgctcaaaggtggtcgggtcgttgcgatgcggttagcggcgggtagcacctcctttcgtgattattcgtaatatacttaggagattcatacgatgatgagcggcgaacacaccctcaaagccgtgcgcggttcttttattgatgtgacccggaccattgataatcccgaagaaattgccagcgccctccgctttattgaagatggcctcctcctcattaaacagggtaaagtggaatggtttggcgaatgggaaaatggtaaacaccaaattcccgataccattcgcgtgcgggattatcggggcaaactcattgtgcccggttttgtggatacccacattcattatccccaaagtgaaatggtgggcgcctacggtgaacaactcctcgaatggctcaataaacatacctttcccaccgaacgccggtatgaagatttggaatacgcccgcgaaatgtccgcctttttcattaaacaactcctccggaatggcaccaccaccgccctcgtgtttggcaccgtgcatccccaaagtgtggatgccctctttgaagccgcctcccatattaatatgcgcatgattgccggcaaagtgatgatggatcggaatgcccccgattatctcctcgataccgccgaaagttcctaccaccaatctaaagaactcattgaacgctggcataaaaatggtcggctcctctatgccattaccccccgttttgcccccaccagctctcccgaacaaatggcgatggcccaacggctcaaagaagaataccccgatacctgggtgcatacccatctctgtgaaaataaagatgaaattgcctgggtgaaaagcctctatcccgatcacgatggctacctcgatgtgtatcatcaatacggcctcaccggtaaaaattgtgtgtttgcccactgtgtgcatctcgaagaaaaagaatgggatcgcctctctgaaaccaaaagttccattgccttttgtcccaccagcaatctctatctcggctctggcctctttaatctcaaaaaagcctggcagaaaaaagtgaaagtgggcatgggcaccgatattggcgccggcaccacctttaatatgctccaaaccctcaatgaagcctataaagtgctccaactccaaggctacagcctctctgcctatgaagccttttacctcgccaccctcggcggtgccaaaagtctcggcctcgatgatctcattggcaattttctccccggtaaagaagccgattttgtggtgatggaacccaccgccacccccctccaacaactccgttatgataatagtgtgtccctcgtggataaactctttgtgatgatgaccctcggcgatgatcgctccatttatcggacctacgtggatggtcgcctcgtgtacgaacggaattaagctagttagaagattcagaccatgcaaaccctcagcattcaacatggcaccctcgtgacgatggatcaatatcgccgggtgctcggcgatagctgggtgcatgtgcaagatggccgcattgtggccctcggtgtgcatgccgaatctgtgcccccccccgccgatcgtgtgattgatgcccgcggtaaagtggtgctccccggttttattaatgcccatacccacgtgaatcaaattctcctccgtggtggtccctctcacggtcgccaactctatgattggctctttaatgtgctctaccccggccaaaaagccatgcgccccgaagatgtggccgtggccgtgcggctctattgtgccgaagccgtgcgcagtggtattaccaccattaatgataatgccgattccgccatttaccccggcaatattgaagccgcgatggccgtgtatggcgaagtgggtgtgcgggtggtgtacgcccgcatgtttttcgatcgcatggatggccggattcaaggttatgtggatgccctcaaagcccggagcccccaagtggaactctgttctattatggaagaaaccgccgtggccaaagatcggattaccgccctcagcgatcaatatcacggcaccgccggtggccgcattagtgtgtggcccgcccccgccattacccccgccgtgaccgtggagggtatgcgttgggcccaagcctttgcccgcgatcgggccgtgatgtggaccctccacatggccgaaagcgatcatgatgaacggctccactggatgtctcccgccgaatatatggaatgttacggcctcctcgatgaacgcctccaagtggcccactgtgtgtattttgatcgcaaagatgtgcggctcctccatcgccacaatgtgaaagtggccagtcaagtggtgtccaatgcctacctcggcagtggtgtggcccccgtgcccgaaatggtggaacgtggcatggccgtgggcattggcaccgatgatggtaattgtaatgattccgtgaatatgattggcgatatgaaatttatggcccatattcaccgggccgtgcatcgcgatgccgatgtgctcacccccgaaaaaattctcgaaatggccaccattgatggcgcccgcagtctcggtatggatcatgaaattggctccattgaaaccggtaaacgggccgatctcattctcctcgatctccgccacccccaaaccaccccccaccatcacctcgccgccaccattgtgtttcaagcctacggtaatgaagtggataccgtgctcattgatggcaatgtggtgatggaaaatcgccggctcagttttctcccccccgaacgggaactcgcctttctcgaagaagcccaaagtcgcgccaccgccattctccaacgcgccaatatggtggccaatcccgcctggcgcagcctctaaaagggtgggcgcgccgacccagctttcttgtacaaactcggccctgcaggagaaggccatcctgacggatggcctttttgaagcttgaaa

SEQ ID NO: 86 - DNA, 7968 bp, Artificial Sequence
  Mel6
  promoter (1)..(89): Pc223 promoter
  gene (104)..(1195): atzD
  gene (1217)..(1942): trzE
  gene (1963)..(3831): DUR1,2
  gene (3853)..(5091): trzC
  gene (5113)..(6432): guaD
  gene (6454)..(7878): triA
  terminator (7929)..(7957): RRNB terminator
ttaacaaaaaagcaggaataaaattaacatgatgtaacagacataagtcccatcaccgttgtataatgttaactgtgggattgcaaaagaggagattaattcaatgtatcacattgatgtgtttcgcattccctgtcatagccccggtgatacctctggcctcgaagatttgattgaaaccggtcgtgtggcccccgccgatattgtggccgtgatgggtaaaaccgagggtaatggctgtgtgaatgattatacccgcgaatacgccaccgccatgctcgccgcctgtctcggccgccacctccaactccccccccatgaagtggaaaaacgggtggcctttgtgatgagtggtggcaccgaaggtgtgctctccccccaccataccgtgtttgcccgtcgccccgccattgatgcccaccgtcccgccggtaaacgtctcaccctcggcattgcctttacccgcgattttctccccgaagaaattggtcggcatgcccaaattaccgaaaccgccggcgccgtgaaacgtgccatgcgtgatgccggtattgccagtattgatgatctccactttgtgcaagtgaaatgtcccctcctcacccccgccaaaattgcctccgcccgcagccggggctgtgcccccgtgaccaccgatacctatgaatctatgggttacagtcggggcgcctccgccctcggtattgccctcgccaccgaagaagtgcccagttccatgctcgtggatgaaagtgtgctcaatgattggtccctcagctctagtctcgcctccgccagcgccggcattgaactcgaacacaatgtggtgattgccattggtatgagtgaacaagccacctccgaactcgtgattgcccacggcgtgatgagcgatgccattgatgccgcctctgtgcgccggaccattgaaagcctcggtattcgctctgatgatgaaatggatcggattgtgaatgtgtttgccaaagccgaagccagtcccgatggtgtggtgcgtggtatgcgtcacaccatgctctctgatagtgatattaattccacccgtcatgcccgtgccgtgaccggtgccgccattgccagcgtggtgggtcacggcatggtgtacgtgtctggcggtgccgaacatcaaggtcccgccggtggtggtccctttgccgtgattgcccgtgcctaagtcaagtaggagattaattccatgatttatagtaccgtgaatgccaatccctatgcctggccctacgatggctccattgatcccgcccacaccgccctcatcctcatcgattggcaaatcgatttttgtggtcccggcggttatgtggatagtatgggctacgatctcagcctcacccgctctggcctcgaacccaccgcccgcgtgctcgccgccgcccgcgataccggcatgaccgtgattcacacccgcgaaggtcatcgtcccgatctcgccgatctcccccccaataaacgttggcggagtgcctccgccggcgccgaaattggcagtgtgggtccctgtggccgcattctcgtgcgtggtgaacccggttgggaaattgtgcccgaagtggccccccgcgaaggtgaacccattattgataaacccggtaaaggtgccttttatgccaccgatctcgatctcctcctccgcacccgcggcattacccacctcattctcaccggcattaccaccgatgtgtgtgtgcataccaccatgcgcgaagccaatgatcggggctacgaatgtctcattctctccgattgtaccggtgccaccgatcggaaacaccatgaagccgccctcagcatggtgaccatgcaaggcggtgtgtttggtgccaccgcccattctgatgatctcctcgccgccctcggcaccaccgtgcccgccgccgccggcccccgtgcccgcaccgaataatctaataggagacaaatactatgaccgtgagttccgataccaccgccgaaatttctctcggctggagtatccaagattggatcgattttcacaaaagctctagttcccaagcctccctccgcctcctcgaatccctcctcgatagccaaaatgtggcccccgtggataatgcctggatttccctcattagcaaggaaaacctcctccatcaatttcaaattctcaagtctcgcgaaaacaaggaaaccctccccctctatggtgtgcccattgccgtgaaagataatattgatgtgcgtggcctccccaccaccgccgcctgtcccagttttgcctacgaaccctctaaagatagtaaagtggtggaactcctccgcaatgccggcgccattattgtgggtaaaaccaatctcgatcaatttgccaccggcctcgtgggcacccgcagtccctatggcaaaaccccctgtgccttttccaaagaacacgtgagcggcggtagctctgccggctccgccagcgtggtggcccgcggtattgtgcccattgccctcggcaccgataccgccggctctggtcgggtgcccgccgccctcaataatctcattggcctcaaacccaccaagggtgtgtttagttgtcaaggcgtggtgcccgcctgtaaatctctcgattgtgtgagtatttttgccctcaatctcagcgatgccgaacgctgttttcggattatgtgtcaacccgatcccgataatgatgaatattcccgcccctacgtgagcaatcccctcaaaaaatttagttccaatgtgaccattgccattcccaaaaatattccctggtatggtgaaaccaaaaatcccgtgctcttttccaatgccgtggaaaatctcagccggaccggcgccaatgtgattgaaattgattttgaacccctcctcgaactcgcccgctgtctctatgaaggcacctgggtggccgaacgctatcaagccattcaatcttttctcgatagtaaaccccccaaagaatctctcgatcccaccgtgattagtatcatcgaaggtgccaaaaaatactccgccgtggattgttttagctttgaatacaaacgccaaggcattctccaaaaagtgcgccggctcctcgaatccgtggatgtgctctgtgtgcccacctgtcccctcaatcccaccatgcaacaagtggccgatgaacccgtgctcgtgaatagccgccaaggcacctggaccaattttgtgaatctcgccgatctcgccgccctcgccgtgcccgccggttttcgggatgatggcctccccaatggtattaccctcattggcaaaaaatttaccgattatgccctcctcgaactcgccaaccgctactttcaaaacatctttcccaacggctcccggacctatggcacctttaccagctctagtgtgaaacccgccaatgatcaactcgtgggccccgattacgatccctctaccagtattaaactcgccgtggtgggcgcccacctcaaaggcctccccctccattggcaactcgaaaaagtgaatgccacctatctctgtaccaccaaaaccagcaaagcctaccaactctttgccctccccaaaaatggtcccgtgctcaaacccggcctccgtcgtgtgcaagattccaatggtagccaaattgaactcgaagtgtattccgtgcccaaagaactctttggcgcctttattagcatggtgcccgaacccctcggcattggttctgtggaactcgaaagtggcgaatggatcaaatcttttatttgtgaagaaagtggctacaaagccaaaggcaccgtggatattaccaaatatggcggttttcgggcctactttgaaatgctctaaattgattaggagatatacaccatgagcatggaaacccactcctatgttgatgttgcgatccgtaacgcccgtctcgccgataccgaaggcattgttgatattctgattcatgatggtcggattgccagcattgtgaaatccaccaaaaccaaaggttccgtggaaattgatgcccatgaaggtctcgtgaccagtggcctcgtggaaccccacattcatctcgataaagccctcaccgccgatcgcgtgcccgccggtagtattggcgatctccggacccgccggggtctcgaaatggccattcgcgccacccgcgatattaaacgcacctttaccgtggaagatgtgcgggaacgcgccattcgggccgccctcatggccagtcgcgccggcaccaccgccctccgcacccatgtggatgtggaccccattgtgggtctcgccggtattcggggcgtgctcgaagcccgcgaagtgtgtgccggtctcattgatattcaaattgtggcctttccccaagaaggcctcttttgtagtgccggtgccgtggatctcatgcgggaagccattaaactcggtgccgatgccgtgggcggtgcccccgccctcgatgatcgcccccaagatcatgtgcgcgccgtgtttgatctcgccgccgaatttggtctccccgtggatatgcacgtggatgaaagtgatcgccgggaagattttaccctcccctttgtgattgaagccgcccgcgaacggcgcgtgcccaatgtgaccgtggcccacatttcctctctcagtgtgcaaaccgatgatgtggcccgctccaccattgccgccctcgccgatgccgatgtgaatgtggtggtgaatcccattattgtgaaaattacccgcctctctgaactcctcgatgccggtgtgtctgtgatgtttggctccgataatctccgcgatcccttttatcccctcggtgccgccaatcccctcggtagtgccatttttgcctgtcaaattgccgccctcggcaccccccaagacctccgccgggtgtttgatgccgtgaccattaatgccgcccgcatgctcggttttcccagtctcctcggtgtggtggaaggtgccgtggccgatctcgccgtgtttcccagtgccacccccgaagaagtggtgctcgatcaacaaagtcccctctttgtgctcaaaggtggtcgggtcgttgcgatgcggttagcggcgggtagcacctcctttcgtgattattcgtaatatacttaggagattcatacgatgatgagcggcgaacacaccctcaaagccgtgcgcggttcttttattgatgtgacccggaccattgataatcccgaagaaattgccagcgccctccgctttattgaagatggcctcctcctcattaaacagggtaaagtggaatggtttggcgaatgggaaaatggtaaacaccaaattcccgataccattcgcgtgcgggattatcggggcaaactcattgtgcccggttttgtggatacccacattcattatccccaaagtgaaatggtgggcgcctacggtgaacaactcctcgaatggctcaataaacatacctttcccaccgaacgccggtatgaagatttggaatacgcccgcgaaatgtccgcctttttcattaaacaactcctccggaatggcaccaccaccgccctcgtgtttggcaccgtgcatccccaaagtgtggatgccctctttgaagccgcctcccatattaatatgcgcatgattgccggcaaagtgatgatggatcggaatgcccccgattatctcctcgataccgccgaaagttcctaccaccaatctaaagaactcattgaacgctggcataaaaatggtcggctcctctatgccattaccccccgttttgcccccaccagctctcccgaacaaatggcgatggcccaacggctcaaagaagaataccccgatacctgggtgcatacccatctctgtgaaaataaagatgaaattgcctgggtgaaaagcctctatcccgatcacgatggctacctcgatgtgtatcatcaatacggcctcaccggtaaaaattgtgtgtttgcccactgtgtgcatctcgaagaaaaagaatgggatcgcctctctgaaaccaaaagttccattgccttttgtcccaccagcaatctctatctcggctctggcctctttaatctcaaaaaagcctggcagaaaaaagtgaaagtgggcatgggcaccgatattggcgccggcaccacctttaatatgctccaaaccctcaatgaagcctataaagtgctccaactccaaggctacagcctctctgcctatgaagccttttacctcgccaccctcggcggtgccaaaagtctcggcctcgatgatctcattggcaattttctccccggtaaagaagccgattttgtggtgatggaacccaccgccacccccctccaacaactccgttatgataatagtgtgtccctcgtggataaactctttgtgatgatgaccctcggcgatgatcgctccatttatcggacctacgtggatggtcgcctcgtgtacgaacggaattaagctagttaggagattcagaccatgcaaaccctcagcattcaacatggcaccctcgtgacgatggatcaatatcgccgggtgctcggcgatagctgggtgcatgtgcaagatggccgcattgtggccctcggtgtgcatgccgaatctgtgcccccccccgccgatcgtgtgattgatgcccgcggtaaagtggtgctccccggttttattaatgcccatacccacgtgaatcaaattctcctccgtggtggtccctctcacggtcgccaactctatgattggctctttaatgtgctctaccccggccaaaaagccatgcgccccgaagatgtggccgtggccgtgcggctctattgtgccgaagccgtgcgcagtggtattaccaccattaatgataatgccgattccgccatttaccccggcaatattgaagccgcgatggccgtgtatggcgaagtgggtgtgcgggtggtgtacgcccgcatgtttttcgatcgcatggatggccggattcaaggttatgtggatgccctcaaagcccggagcccccaagtggaactctgttctattatggaagaaaccgccgtggccaaagatcggattaccgccctcagcgatcaatatcacggcaccgccggtggccgcattagtgtgtggcccgcccccgccattacccccgccgtgaccgtggagggtatgcgttgggcccaagcctttgcccgcgatcgggccgtgatgtggaccctccacatggccgaaagcgatcatgatgaacggctccactggatgtctcccgccgaatatatggaatgttacggcctcctcgatgaacgcctccaagtggcccactgtgtgtattttgatcgcaaagatgtgcggctcctccatcgccacaatgtgaaagtggccagtcaagtggtgtccaatgcctacctcggcagtggtgtggcccccgtgcccgaaatggtgaaacgtggcatggccgtgggcattggcaccgatgatggtaattgtaatgattccgtgaatatgattggcgatatgaaatttatggcccatattcaccgggccgtgcatcgcgatgccgatgtgctcacccccgaaaaaattctcgaaatggccaccattgatggcgcccgcagtctcggtatggatcatgaaattggctccattgaaaccggtaaacgggccgatctcattctcctcgatctccgccacccccaaaccaccccccaccatcacctcgccgccaccattgtgtttcaagcctacggtaatgaagtggataccgtgctcattgatggcaatgtggtgatggaaaatcgccggctcagttttctcccccccgaacgggaactcgcctttctcgaagaagcccaaagtcgcgccaccgccattctccaacgcgccaatatggtggccaatcccgcctggcgcagcctctaaaagggtgggcgcgccgacccagctttcttgtacaaactcggccctgcaggagaaggccatcctgacggatggcctttttgaagcttgaaa

SEQ ID NO: 87 - DNA, 7968 bp, Artificial Sequence
  Mel7
  promoter (1)..(89): Pc223 promoter
  gene (104)..(1195): atzD
  gene (1217)..(1942): trzE
  gene (1963)..(3831): DUR1,2
  gene (3853)..(5091): trzC
  gene (5113)..(6432): guaD
  gene (6454)..(7878): triA
  terminator (7929)..(7957): RRNB terminator
ttaacaaaaaagcaggaataaaattaacatgatgtaacagacataagtcccatcaccgttgtataatgttaactgtgggattgcaaaagaggagattaattcaatgtatcacattgatgtgtttcgcattccctgtcatagccccggtgatacctctggcctcgaagatttgattgaaaccggtcgtgtggcccccgccgatattgtggccgtgatgggtaaaaccgagggtaatggctgtgtgaatgattatacccgcgaatacgccaccgccatgctcgccgcctgtctcggccgccacctccaactccccccccatgaagtggaaaaacgggtggcctttgtgatgagtggtggcaccgaaggtgtgctctccccccaccataccgtgtttgcccgtcgccccgccattgatgcccaccgtcccgccggtaaacgtctcaccctcggcattgcctttacccgcgattttctccccgaagaaattggtcggcatgcccaaattaccgaaaccgccggcgccgtgaaacgtgccatgcgtgatgccggtattgccagtattgatgatctccactttgtgcaagtgaaatgtcccctcctcacccccgccaaaattgcctccgcccgcagccggggctgtgcccccgtgaccaccgatacctatgaatctatgggttacagtcggggcgcctccgccctcggtattgccctcgccaccgaagaagtgcccagttccatgctcgtggatgaaagtgtgctcaatgattggtccctcagctctagtctcgcctccgccagcgccggcattgaactcgaacacaatgtggtgattgccattggtatgagtgaacaagccacctccgaactcgtgattgcccacggcgtgatgagcgatgccattgatgccgcctctgtgcgccggaccattgaaagcctcggtattcgctctgatgatgaaatggatcggattgtgaatgtgtttgccaaagccgaagccagtcccgatggtgtggtgcgtggtatgcgtcacaccatgctctctgatagtgatattaattccacccgtcatgcccgtgccgtgaccggtgccgccattgccagcgtggtgggtcacggcatggtgtacgtgtctggcggtgccgaacatcaaggtcccgccggtggtggtccctttgccgtgattgcccgtgcctaagtcaagtaggagattaattccatgatttatagtaccgtgaatgccaatccctatgcctggccctacgatggctccattgatcccgcccacaccgccctcatcctcatcgattggcaaatcgatttttgtggtcccggcggttatgtggatagtatgggctacgatctcagcctcacccgctctggcctcgaacccaccgcccgcgtgctcgccgccgcccgcgataccggcatgaccgtgattcacacccgcgaaggtcatcgtcccgatctcgccgatctcccccccaataaacgttggcggagtgcctccgccggcgccgaaattggcagtgtgggtccctgtggccgcattctcgtgcgtggtgaacccggttgggaaattgtgcccgaagtggccccccgcgaaggtgaacccattattgataaacccggtaaaggtgccttttatgccaccgatctcgatctcctcctccgcacccgcggcattacccacctcattctcaccggcattaccaccgatgtgtgtgtgcataccaccatgcgcgaagccaatgatcggggctacgaatgtctcattctctccgattgtaccggtgccaccgatcggaaacaccatgaagccgccctcagcatggtgaccatgcaaggcggtgtgtttggtgccaccgcccattctgatgatctcctcgccgccctcggcaccaccgtgcccgccgccgccggcccccgtgcccgcaccgaataatctaataggagacaaatactatgaccgtgagttccgataccaccgccgaaatttctctcggctggagtatccaagattggatcgattttcacaaaagctctagttcccaagcctccctccgcctcctcgaatccctcctcgatagccaaaatgtggcccccgtggataatgcctggatttccctcattagcaaggaaaacctcctccatcaatttcaaattctcaagtctcgcgaaaacaaggaaaccctccccctctatggtgtgcccattgccgtgaaagataatattgatgtgcgtggcctccccaccaccgccgcctgtcccagttttgcctacgaaccctctaaagatagtaaagtggtggaactcctccgcaatgccggcgccattattgtgggtaaaaccaatctcgatcaatttgccaccggcctcgtgggcacccgcagtccctatggcaaaaccccctgtgccttttccaaagaacacgtgagcggcggtagctctgccggctccgccagcgtggtggcccgcggtattgtgcccattgccctcggcaccgataccgccggctctggtcgggtgcccgccgccctcaataatctcattggcctcaaacccaccaagggtgtgtttagttgtcaaggcgtggtgcccgcctgtaaatctctcgattgtgtgagtatttttgccctcaatctcagcgatgccgaacgctgttttcggattatgtgtcaacccgatcccgataatgatgaatattcccgcccctacgtgagcaatcccctcaaaaaatttagttccaatgtgaccattgccattcccaaaaatattccctggtatggtgaaaccaaaaatcccgtgctcttttccaatgccgtggaaaatctcagccggaccggcgccaatgtgattgaaattgattttgaacccctcctcgaactcgcccgctgtctctatgaaggcacctgggtggccgaacgctatcaagccattcaatcttttctcgatagtaaaccccccaaagaatctctcgatcccaccgtgattagtatcatcgaaggtgccaaaaaatactccgccgtggattgttttagctttgaatacaaacgccaaggcattctccaaaaagtgcgccggctcctcgaatccgtggatgtgctctgtgtgcccacctgtcccctcaatcccaccatgcaacaagtggccgatgaacccgtgctcgtgaatagccgccaaggcacctggaccaattttgtgaatctcgccgatctcgccgccctcgccgtgcccgccggttttcgggatgatggcctccccaatggtattaccctcattggcaaaaaatttaccgattatgccctcctcgaactcgccaaccgctactttcaaaacatctttcccaacggctcccggacctatggcacctttaccagctctagtgtgaaacccgccaatgatcaactcgtgggccccgattacgatccctctaccagtattaaactcgccgtggtgggcgcccacctcaaaggcctccccctccattggcaactcgaaaaagtgaatgccacctatctctgtaccaccaaaaccagcaaagcctaccaactctttgccctccccaaaaatggtcccgtgctcaaacccggcctccgtcgtgtgcaagattccaatggtagccaaattgaactcgaagtgtattccgtgcccaaagaactctttggcgcctttattagcatggtgcccgaacccctcggcattggttctgtggaactcgaaagtggcgaatggatcaaatcttttatttgtgaagaaagtggctacaaagccaaaggcaccgtggatattaccaaatatggcggttttcgggcctactttgaaatgctctaaattgattaggagatatacaccatgagcatggaaacccactcctatgttgatgttgcgatccgtaacgcccgtctcgccgataccgaaggcattgttgatattctgattcatgatggtcggattgccagcattgtgaaatccaccaaaaccaaaggttccgtggaaattgatgcccatgaaggtctcgtgaccagtggcctcgtggaaccccacattcatctcgataaagccctcaccgccgatcgcgtgcccgccggtagtattggcgatctccggacccgccggggtctcgaaatggccattcgcgccacccgcgatattaaacgcacctttaccgtggaagatgtgcgggaacgcgccattcgggccgccctcatggccagtcgcgccggcaccaccgccctccgcacccatgtggatgtggaccccattgtgggtctcgccggtattcggggcgtgctcgaagcccgcgaagtgtgtgccggtctcattgatattcaaattgtggcctttccccaagaaggcctcttttgtagtgccggtgccgtggatctcatgcgggaagccattaaactcggtgccgatgccgtgggcggtgcccccgccctcgatgatcgcccccaagatcatgtgcgcgccgtgtttgatctcgccgccgaatttggtctccccgtggatatgcacgtggatgaaagtgatcgccgggaagattttaccctcccctttgtgattgaagccgcccgcgaacggcgcgtgcccaatgtgaccgtggcccacatttcctctctcagtgtgcaaaccgatgatgtggcccgctccaccattgccgccctcgccgatgccgatgtgaatgtggtggtgaatcccattattgtgaaaattacccgcctctctgaactcctcgatgccggtgtgtctgtgatgtttggctccgataatctccgcgatcccttttatcccctcggtgccgccaatcccctcggtagtgccatttttgcctgtcaaattgccgccctcggcaccccccaagacctccgccgggtgtttgatgccgtgaccattaatgccgcccgcatgctcggttttcccagtctcctcggtgtggtggaaggtgccgtggccgatctcgccgtgtttcccagtgccacccccgaagaagtggtgctcgatcaacaaagtcccctctttgtgctcaaaggtggtcgggtcgttgcgatgcggttagcggcgggtagcacctcctttcgtgattattcgtaatatacttaggagattcatacgatgatgagcggcgaacacaccctcaaagccgtgcgcggttcttttattgatgtgacccggaccattgataatcccgaagaaattgccagcgccctccgctttattgaagatggcctcctcctcattaaacagggtaaagtggaatggtttggcgaatgggaaaatggtaaacaccaaattcccgataccattcgcgtgcgggattatcggggcaaactcattgtgcccggttttgtggatacccacattcattatccccaaagtgaaatggtgggcgcctacggtgaacaactcctcgaatggctcaataaacatacctttcccaccgaacgccggtatgaagatttggaatacgcccgcgaaatgtccgcctttttcattaaacaactcctccggaatggcaccaccaccgccctcgtgtttggcaccgtgcatccccaaagtgtggatgccctctttgaagccgcctcccatattaatatgcgcatgattgccggcaaagtgatgatggatcggaatgcccccgattatctcctcgataccgccgaaagttcctaccaccaatctaaagaactcattgaacgctggcataaaaatggtcggctcctctatgccattaccccccgttttgcccccaccagctctcccgaacaaatggcgatggcccaacggctcaaagaagaataccccgatacctgggtgcatacccatctctgtgaaaataaagatgaaattgcctgggtgaaaagcctctatcccgatcacgatggctacctcgatgtgtatcatcaatacggcctcaccggtaaaaattgtgtgtttgcccactgtgtgcatctcgaagaaaaagaatgggatcgcctctctgaaaccaaaagttccattgccttttgtcccaccagcaatctctatctcggctctggcctctttaatctcaaaaaagcctggcagaaaaaagtgaaagtgggcatgggcaccgatattggcgccggcaccacctttaatatgctccaaaccctcaatgaagcctataaagtgctccaactccaaggctacagcctctctgcctatgaagccttttacctcgccaccctcggcggtgccaaaagtctcggcctcgatgatctcattggcaattttctccccggtaaagaagccgattttgtggtgatggaacccaccgccacccccctccaacaactccgttatgataatagtgtgtccctcgtggataaactctttgtgatgatgaccctcggcgatgatcgctccatttatcggacctacgtggatggtcgcctcgtgtacgaacggaattaagctagttaggagattcagaccatgcaaaccctcagcattcaacatggcaccctcgtgacgatggatcaatatcgccgggtgctcggcgatagctgggtgcatgtgcaagatggccgcattgtggccctcggtgtgcatgccgaatctgtgcccccccccgccgatcgtgtgattgatgcccgcggtaaagtggtgctccccggttttattaatgcccatacccacgtgaatcaaattctcctccgtggtggtccctctcacggtcgccaactctatgattggctctttaatgtgctctaccccggccaaaaagccatgcgccccgaagatgtggccgtggccgtgcggctctattgtgccgaagccgtgcgcagtggtattaccaccattaatgataatgccgattccgccatttaccccggcaatattgaagccgcgatggccgtgtatggcgaagtgggtgtgcgggtggtgtacgcccgcatgtttttcgatcgcatggatggccggattcaaggttatgtggatgccctcaaagcccggagcccccaagtggaactctgttctattatggaagaaaccgccgtggccaaagatcggattaccgccctcagcgatcaatatcacggcaccgccggtggccgcattagtgtgtggcccgcccccgccattacccccgccgtgaccgtggagggtatgcgttgggcccaagcctttgcccgcgatcgggccgtgatgtggaccctccacatggccgaaagcgatcatgatgaacggctctactggatgtctcccgccgaatatatggaatgttacggcctcctcgatgaacgcctccaagtggcccactgtgtgtattttgatcgcaaagatgtgcggctcctccatcgccacaatgtgaaagtggccagtcaagtggtgtccaatgcctacctcggcagtggtgtggcccccgtgcccgaaatggtggaacgtggcatggccgtgggcattggcaccgatgatggtaattgtaatgattccgtgaatatgattggcgatatgaaatttatggcccatattcaccgggccgtgcatcgcgatgccgatgtgctcacccccgaaaaaattctcgaaatggccaccattgatggcgcccgcagtctcggtatggatcatgaaattggctccattgaaaccggtaaacgggccgatctcattctcctcgatctccgccacccccaaaccaccccccaccatcacctcgccgccaccattgtgtttcaagcctacggtaatgaagtggataccgtgctcattgatggcaatgtggtgatggaaaatcgccggctcagttttctcccccccgaacgggaactcgcctttctcgaagaagcccaaagtcgcgccaccgccattctccaacgcgccaatatggtggccaatcccgcctggcgcagcctctaaaagggtgggcgcgccgacccagctttcttgtacaaactcggccctgcaggagaaggccatcctgacggatggcctttttgaagcttgaaa

SEQ ID NO: 88 - DNA, 7968 bp, Artificial Sequence
  Mel8
  promoter (1)..(89): Pc223 promoter
  gene (104)..(1195): atzD
  gene (1217)..(1942): trzE
  gene (1963)..(3831): DUR1,2
  gene (3853)..(5091): trzC
  gene (5113)..(6432): guaD
  gene (6454)..(7878): triA
  terminator (7929)..(7957): RRNB terminator
ttaacaaaaaagcaggaataaaattaacatgatgtaacagacataagtcccatcaccgttgtataatgttaactgtgggattgcaaaagaggagattaattcaatgtatcacattgatgtgtttcgcattccctgtcatagccccggtgatacctctggcctcgaagatttgattgaaaccggtcgtgtggcccccgccgatattgtggccgtgatgggtaaaaccgagggtaatggctgtgtgaatgattatacccgcgaatacgccaccgccatgctcgccgcctgtctcggccgccacctccaactccccccccatgaagtggaaaaacgggtggcctttgtgatgagtggtggcaccgaaggtgtgctctccccccaccataccgtgtttgcccgtcgccccgccattgatgcccaccgtcccgccggtaaacgtctcaccctcggcattgcctttacccgcgattttctccccgaagaaattggtcggcatgcccaaattaccgaaaccgccggcgccgtgaaacgtgccatgcgtgatgccggtattgccagtattgatgatctccactttgtgcaagtgaaatgtcccctcctcacccccgccaaaattgcctccgcccgcagccggggctgtgcccccgtgaccaccgatacctatgaatctatgggttacagtcggggcgcctccgccctcggtattgccctcgccaccgaagaagtgcccagttccatgctcgtggatgaaagtgtgctcaatgattggtccctcagctctagtctcgcctccgccagcgccggcattgaactcgaacacaatgtggtgattgccattggtatgagtgaacaagccacctccgaactcgtgattgcccacggcgtgatgagcgatgccattgatgccgcctctgtgcgccggaccattgaaagcctcggtattcgctctgatgatgaaatggatcggattgtgaatgtgtttgccaaagccgaagccagtcccgatggtgtggtgcgtggtatgcgtcacaccatgctctctgatagtgatattaattccacccgtcatgcccgtgccgtgaccggtgccgccattgccagcgtggtgggtcacggcatggtgtacgtgtctggcggtgccgaacatcaaggtcccgccggtggtggtccctttgccgtgattgcccgtgcctaagtcaagtaggagattaattccatgatttatagtaccgtgaatgccaatccctatgcctggccctacgatggctccattgatcccgcccacaccgccctcatcctcatcgattggcaaatcgatttttgtggtcccggcggttatgtggatagtatgggctacgatctcagcctcacccgctctggcctcgaacccaccgcccgcgtgctcgccgccgcccgcgataccggcatgaccgtgattcacacccgcgaaggtcatcgtcccgatctcgccgatctcccccccaataaacgttggcggagtgcctccgccggcgccgaaattggcagtgtgggtccctgtggccgcattctcgtgcgtggtgaacccggttgggaaattgtgcccgaagtggccccccgcgaaggtgaacccattattgataaacccggtaaaggtgccttttatgccaccgatctcgatctcctcctccgcacccgcggcattacccacctcattctcaccggcattaccaccgatgtgtgtgtgcataccaccatgcgcgaagccaatgatcggggctacgaatgtctcattctctccgattgtaccggtgccaccgatcggaaacaccatgaagccgccctcagcatggtgaccatgcaaggcggtgtgtttggtgccaccgcccattctgatgatctcctcgccgccctcggcaccaccgtgcccgccgccgccggcccccgtgcccgcaccgaataatctaataggagacaaatactatgaccgtgagttccgataccaccgccgaaatttctctcggctggagtatccaagattggatcgattttcacaaaagctctagttcccaagcctccctccgcctcctcgaatccctcctcgatagccaaaatgtggcccccgtggataatgcctggatttccctcattagcaaggaaaacctcctccatcaatttcaaattctcaagtctcgcgaaaacaaggaaaccctccccctctatggtgtgcccattgccgtgaaagataatattgatgtgcgtggcctccccaccaccgccgcctgtcccagttttgcctacgaaccctctaaagatagtaaagtggtggaactcctccgcaatgccggcgccattattgtgggtaaaaccaatctcgatcaatttgccaccggcctcgtgggcacccgcagtccctatggcaaaaccccctgtgccttttccaaagaacacgtgagcggcggtagctctgccggctccgccagcgtggtggcccgcggtattgtgcccattgccctcggcaccgataccgccggctctggtcgggtgcccgccgccctcaataatctcattggcctcaaacccaccaagggtgtgtttagttgtcaaggcgtggtgcccgcctgtaaatctctcgattgtgtgagtatttttgccctcaatctcagcgatgccgaacgctgttttcggattatgtgtcaacccgatcccgataatgatgaatattcccgcccctacgtgagcaatcccctcaaaaaatttagttccaatgtgaccattgccattcccaaaaatattccctggtatggtgaaaccaaaaatcccgtgctcttttccaatgccgtggaaaatctcagccggaccggcgccaatgtgattgaaattgattttgaacccctcctcgaactcgcccgctgtctctatgaaggcacctgggtggccgaacgctatcaagccattcaatcttttctcgatagtaaaccccccaaagaatctctcgatcccaccgtgattagtatcatcgaaggtgccaaaaaatactccgccgtggattgttttagctttgaatacaaacgccaaggcattctccaaaaagtgcgccggctcctcgaatccgtggatgtgctctgtgtgcccacctgtcccctcaatcccaccatgcaacaagtggccgatgaacccgtgctcgtgaatagccgccaaggcacctggaccaattttgtgaatctcgccgatctcgccgccctcgccgtgcccgccggttttcgggatgatggcctccccaatggtattaccctcattggcaaaaaatttaccgattatgccctcctcgaactcgccaaccgctactttcaaaacatctttcccaacggctcccggacctatggcacctttaccagctctagtgtgaaacccgccaatgatcaactcgtgggccccgattacgatccctctaccagtattaaactcgccgtggtgggcgcccacctcaaaggcctccccctccattggcaactcgaaaaagtgaatgccacctatctctgtaccaccaaaaccagcaaagcctaccaactctttgccctccccaaaaatggtcccgtgctcaaacccggcctccgtcgtgtgcaagattccaatggtagccaaattgaactcgaagtgtattccgtgcccaaagaactctttggcgcctttattagcatggtgcccgaacccctcggcattggttctgtggaactcgaaagtggcgaatggatcaaatcttttatttgtgaagaaagtggctacaaagccaaaggcaccgtggatattaccaaatatggcggttttcgggcctactttgaaatgctctaaattgattaggagatatacaccatgagcatggaaacccactcctatgttgatgttgcgatccgtaacgcccgtctcgccgataccgaaggcattgttgatattctgattcatgatggtcggattgccagcattgtgaaatccaccaaaaccaaaggttccgtggaaattgatgcccatgaaggtctcgtgaccagtggcctcgtggaaccccacattcatctcgataaagccctcaccgccgatcgcgtgcccgccggtagtattggcgatctccggacccgccggggtctcgaaatggccattcgcgccacccgcgatattaaacgcacctttaccgtggaagatgtgcgggaacgcgccattcgggccgccctcatggccagtcgcgccggcaccaccgccctccgcacccatgtggatgtggaccccattgtgggtctcgccggtattcggggcgtgctcgaagcccgcgaagtgtgtgccggtctcattgatattcaaattgtggcctttccccaagaaggcctcttttgtagtgccggtgccgtggatctcatgcgggaagccattaaactcggtgccgatgccgtgggcggtgcccccgccctcgatgatcgcccccaagatcatgtgcgcgccgtgtttgatctcgccgccgaatttggtctccccgtggatatgcacgtggatgaaagtgatcgccgggaagattttaccctcccctttgtgattgaagccgcccgcgaacggcgcgtgcccaatgtgaccgtggcccacatttcctctctcagtgtgcaaaccgatgatgtggcccgctccaccattgccgccctcgccgatgccgatgtgaatgtggtggtgaatcccattattgtgaaaattacccgcctctctgaactcctcgatgccggtgtgtctgtgatgtttggctccgataatctccgcgatcccttttatcccctcggtgccgccaatcccctcggtagtgccatttttgcctgtcaaattgccgccctcggcaccccccaagacctccgccgggtgtttgatgccgtgaccattaatgccgcccgcatgctcggttttcccagtctcctcggtgtggtggaaggtgccgtggccgatctcgccgtgtttcccagtgccacccccgaagaagtggtgctcgatcaacaaagtcccctctttgtgctcaaaggtggtcgggtcgttgcgatgcggttagcggcgggtagcacctcctttcgtgattattcgtaatatacttaggagattcatacgatgatgagcggcgaacacaccctcaaagccgtgcgcggttcttttattgatgtgacccggaccattgataatcccgaagaaattgccagcgccctccgctttattgaagatggcctcctcctcattaaacagggtaaagtggaatggtttggcgaatgggaaaatggtaaacaccaaattcccgataccattcgcgtgcgggattatcggggcaaactcattgtgcccggttttgtggatacccacattcattatccccaaagtgaaatggtgggcgcctacggtgaacaactcctcgaatggctcaataaacatacctttcccaccgaacgccggtatgaagatttggaatacgcccgcgaaatgtccgcctttttcattaaacaactcctccggaatggcaccaccaccgccctcgtgtttggcaccgtgcatccccaaagtgtggatgccctctttgaagccgcctcccatattaatatgcgcatgattgccggcaaagtgatgatggatcggaatgcccccgattatctcctcgataccgccgaaagttcctaccaccaatctaaagaactcattgaacgctggcataaaaatggtcggctcctctatgccattaccccccgttttgcccccaccagctctcccgaacaaatggcgatggcccaacggctcaaagaagaataccccgatacctgggtgcatacccatctctgtgaaaataaagatgaaattgcctgggtgaaaagcctctatcccgatcacgatggctacctcgatgtgtatcatcaatacggcctcaccggtaaaaattgtgtgtttgcccactgtgtgcatctcgaagaaaaagaatgggatcgcctctctgaaaccaaaagttccattgccttttgtcccaccagcaatctctatctcggctctggcctctttaatctcaaaaaagcctggcagaaaaaagtgaaagtgggcatgggcaccgatattggcgccggcaccacctttaatatgctccaaaccctcaatgaagcctataaagtgctccaactccaaggctacagcctctctgcctatgaagccttttacctcgccaccctcggcggtgccaaaagtctcggcctcgatgatctcattggcaattttctccccggtaaagaagccgattttgtggtgatggaacccaccgccacccccctccaacaactccgttatgataatagtgtgtccctcgtggataaactctttgtgatgatgaccctcggcgatgatcgctccatttatcggacctacgtggatggtcgcctcgtgtacgaacggaattaagctagttaggagattcagaccatgcaaaccctcagcattcaacatggcaccctcgtgacgatggatcaatatcgccgggtgctcggcgatagctgggtgcatgtgcaagatggccgcattgtggccctcggtgtgcatgccgaatctgtgcccccccccgccgatcgtgtgattgatgcccgcggtaaagtggtgctccccggttttattaatgcccatacccacgtgaatcaaattctcctccgtggtggtccctctcacggtcgccaactctatgattggctctttaatgtgctctaccccggccaaaaagccatgcgccccgaagatgtggccgtggccgtgcggctctattgtgccgaagccgtgcgcagtggtattaccaccattaatgataatgccgattccgccatttaccccggcaatattgaagccgcgatggccgtgtatggcgaagtgggtgtgcgggtggtgtacgcccgcatgtttttcgatcgcatggatggccggattcaaggttatgtggatgccctcaaagcccggagcccccaagtggaactctgttctattatggaagaaaccgccgtggccaaagatcggattaccgccctcagcgatcaatatcacggcaccgccggtggccgcattagtgtgtggcccgcccccgccattacccccgccgtgaccgtggagggtatgcgttgggcccaagcctttgcccgcgatcgggccgtgatgtggaccctccacatggccgaaagcgatcatgatgaacggctccactggatgtctcccgccgaatatatggaatgttacggcctcctcgatgaacgcctccaagtggcccactgtgtgtattttgatcgcaaagatgtgcggctcctccatcgccacaatgtgaaagtggccagtcaagtggtgtccaatgcctacctcggcagtggtgtggcccccgtgcccgaaatggtggaacgtggcatggccgtgggcattggcaccgatgatggtaattgtaatgattccgtgaatatgattggcgatatgaaatttatggcccatattcaccgggccgtgcatcgcgatgtcgatgtgctcacccccgaaaaaattctcgaaatggccaccattgatggcgcccgcagtctcggtatggatcatgaaattggctccattgaaaccggtaaacgggccgatctcattctcctcgatctccgccacccccaaaccaccccccaccatcacctcgccgccaccattgtgtttcaagcctacggtaatgaagtggataccgtgctcattgatggcaatgtggtgatggaaaatcgccggctcagttttctcccccccgaacgggaactcgcctttctcgaagaagcccaaagtcgcgccaccgccattctccaacgcgccaatatggtggccaatcccgcctggcgcagcctctaaaagggtgggcgcgccgacccagctttcttgtacaaactcggccctgcaggagaaggccatcctgacggatggcctttttgaagcttgaaa

SEQ ID NO: 89 - DNA, 1011 bp, Artificial Sequence
  ptxD original sequence
atgctgccgaaactcgttataactcaccgagtacacgatgagatcctgcaactgctggcgccacattgcgagctgatgaccaaccagaccgacagcacgctgacgcgcgaggaaattctgcgccgctgtcgcgatgctcaggcgatgatggcgttcatgcccgatcgggtcgatgcagactttcttcaagcctgccctgagctgcgtgtagtcggctgcgcgctcaagggcttcgacaatttcgatgtggacgcctgtactgcccgcggggtctggctgaccttcgtgcctgatctgttgacggtcccgactgccgagctggcgatcggactggcggtggggctggggcggcatctgcgggcagcagatgcgttcgtccgctctggcgagttccagggctggcaaccacagttctacggcacggggctggataacgctacggtcggcatccttggcatgggcgccatcggactggccatggctgatcgcttgcagggatggggcgcgaccctgcagtaccacgaggcgaaggctctggatacacaaaccgagcaacggctcggcctgcgccaggtggcgtgcagcgaactcttcgccagctcggacttcatcctgctggcgcttcccttgaatgccgatacccagcatctggtcaacgccgagctgcttgccctcgtacggccgggcgctctgcttgtaaacccctgtcgtggttcggtagtggatgaagccgccgtgctcgcggcgcttgagcgaggccagctcggcgggtatgcggcggatgtattcgaaatggaagactgggctcgcgcggaccggccgcggctgatcgatcctgcgctgctcgcgcatccgaatacgctgttcactccgcacatagggtcggcagtgcgcgcggtgcgcctggagattgaacgttgtgcagcgcagaacatcatccaggtattggcaggtgcgcgcccaatcaacgctgcgaaccgtctgcccaaggccgagcctgccgcatgttga

SEQ ID NO: 90 - DNA, 1011 bp, Artificial Sequence
  ptxD codon optimized
atgctgcccaaactggtcatcacccaccgcgtccatgatgaaattctgcagctgctggccccgcactgtgagctgatgaccaaccaaaccgatagcaccctgacgcgcgaagagatcctgcgccgctgccgcgatgctcaggccatgatggcttttatgcccgatcgcgtggatgcggatttcctgcaagcttgtcccgaactgcgcgtggtgggttgtgccctgaagggttttgataattttgatgtggatgcctgcacggcccgcggcgtgtggctgacctttgtccccgatctgctgaccgtgccgaccgctgaactggccattggcctggcggtgggtctgggtcgccatctgcgcgcggccgatgcgtttgtgcgcagcggcgagttccagggttggcagccccaattctatggcaccggcctggataatgccacggtgggcatcctgggtatgggtgctattggcctggccatggctgatcgcctgcagggttggggcgccaccctgcaatatcacgaagccaaagcgctggatacgcaaacggaacagcgcctgggtctgcgccaggtggcttgcagcgaactgtttgccagcagcgatttcatcctgctggccctgccgctgaacgcggatacccaacatctggtcaatgctgaactgctggctctggtgcgccccggtgctctgctggtcaatccgtgtcgcggcagcgtggtggatgaagcggctgtgctggccgctctggagcgcggtcaactgggcggctacgccgcggatgtctttgaaatggaggattgggctcgcgcggatcgcccacgcctgatcgatcccgccctgctggctcacccaaacaccctgttcaccccgcatattggcagcgccgtgcgcgcggtccgcctggaaattgagcgctgcgctgcccagaatatcattcaagtgctggccggtgctcgccccattaacgccgctaatcgcctgcccaaggccgaaccggccgcgtgctag

SEQ ID NO: 91 - DNA, 1011 bp, Artificial Sequence
  ptxD codon optimized NADP+
atgctgcccaaactggtcatcacccaccgcgtccatgatgaaattctgcagctgctggccccgcactgtgagctgatgaccaaccaaaccgatagcaccctgacgcgcgaagagatcctgcgccgctgccgcgatgctcaggccatgatggcttttatgcccgatcgcgtggatgcggatttcctgcaagcttgtcccgaactgcgcgtggtgggttgtgccctgaagggttttgataattttgatgtggatgcctgcacggcccgcggcgtgtggctgacctttgtccccgatctgctgaccgtgccgaccgctgaactggccattggcctggcggtgggtctgggtcgccatctgcgcgcggccgatgcgtttgtgcgcagcggcgagttccagggttggcagccccaattctatggcaccggcctggataatgccacggtgggcatcctgggtatgggtgctattggcctggccatggctgatcgcctgcagggttggggcgccaccctgcaatatcacgctcgtaaagcgctggatacgcaaacggaacagcgcctgggtctgcgccaggtggcttgcagcgaactgtttgccagcagcgatttcatcctgctggccctgccgctgaacgcggatacccaacatctggtcaatgctgaactgctggctctggtgcgccccggtgctctgctggtcaatccgtgtcgcggcagcgtggtggatgaagcggctgtgctggccgctctggagcgcggtcaactgggcggctacgccgcggatgtctttgaaatggaggattgggctcgcgcggatcgcccacgcctgatcgatcccgccctgctggctcacccaaacaccctgttcaccccgcatattggcagcgccgtgcgcgcggtccgcctggaaattgagcgctgcgctgcccagaatatcattcaagtgctggccggtgctcgccccattaacgccgctaatcgcctgcccaaggccgaaccggccgcgtgctag

SEQ ID NO: 92 - DNA, 61 bp, Artificial Sequence
  psbA promoter
gatctcaatgaatattggttgacacgggcgtataagacatgttatactgttgaataacaag

SEQ ID NO: 93 - DNA, 1236 bp, Artificial Sequence
  ptxD operon
  promoter (1)..(61): psbA promoter
  gene (84)..(1091): ptxD
  terminator (1107)..(1194)
gatctcaatgaatattggttgacacgggcgtataagacatgttatactgttgaataacaagactagatagtggaggtactagaatgctgcccaaactggtcatcacccaccgcgtccatgatgaaattctgcagctgctggccccgcactgtgagctgatgaccaaccaaaccgatagcaccctgacgcgcgaagagatcctgcgccgctgccgcgatgctcaggccatgatggcttttatgcccgatcgcgtggatgcggatttcctgcaagcttgtcccgaactgcgcgtggtgggttgtgccctgaagggttttgataattttgatgtggatgcctgcacggcccgcggcgtgtggctgacctttgtccccgatctgctgaccgtgccgaccgctgaactggccattggcctggcggtgggtctgggtcgccatctgcgcgcggccgatgcgtttgtgcgcagcggcgagttccagggttggcagccccaattctatggcaccggcctggataatgccacggtgggcatcctgggtatgggtgctattggcctggccatggctgatcgcctgcagggttggggcgccaccctgcaatatcacgaagccaaagcgctggatacgcaaacggaacagcgcctgggtctgcgccaggtggcttgcagcgaactgtttgccagcagcgatttcatcctgctggccctgccgctgaacgcggatacccaacatctggtcaatgctgaactgctggctctggtgcgccccggtgctctgctggtcaatccgtgtcgcggcagcgtggtggatgaagcggctgtgctggccgctctggagcgcggtcaactgggcggctacgccgcggatgtctttgaaatggaggattgggctcgcgcggatcgcccacgcctgatcgatcccgccctgctggctcacccaaacaccctgttcaccccgcatattggcagcgccgtgcgcgcggtccgcctggaaattgagcgctgcgctgcccagaatatcattcaagtgctggccggtgctcgccccattaacgccgctaatcgcctgcccaaggccgaaccggccgcgtgctagttaaacctcagcggtttagtgaccgactaacacttttctcataaaatcccagggaggtttcggcctccctttttttcacttgctaagctctctttcgtttgattgtctgacttggttcacgtagaaaaaccagaagggacgc

SEQ ID NO: 94 - DNA, 1443 bp, Artificial Sequence
  pSJ051
  RBS (5)..(10)
  gene (19)..(1443): triA gene sequence
agttaggagattcagaccatgcaaacgctcagcatccagcacggtaccctcgtcacgatggatcagtaccgcagagtccttggggatagctgggttcacgtgcaggatggacggatcgtcgcgctcggagtgcacgccgagtcggtgcctccgccagcggatcgggtgatcgatgcacgcggcaaggtcgtgttacccggtttcatcaatgcccacacccatgtgaaccagatcctcctgcgcggagggccctcgcacgggcgtcaactctatgactggctgttcaacgttttgtatccgggacaaaaggcgatgagaccggaggacgtagcggtggcggtgaggttgtattgtgcggaagctgtgcgcagcgggattacgacgatcaacgacaacgccgattcggccatctacccaggcaacatcgaggccgcgatggcggtctatggtgaggtgggtgtgagggtcgtctacgcccgcatgttctttgatcggatggacgggcgcattcaagggtatgtggacgccttgaaggctcgctctccccaagtcgaactgtgctcgatcatggaggaaacggctgtggccaaagatcggatcacagccctgtcagatcagtatcatggcacggcaggaggtcgtatatcagtttggcccgctcctgccattaccccggcggtgacagttgaaggaatgcgatgggcacaagccttcgcccgtgatcgggcggtaatgtggacgcttcacatggcggagagcgatcatgatgagcggcttcattggatgagtcccgccgagtacatggagtgttacggactcttggatgagcgtctgcaggtcgcgcattgcgtgtactttgaccggaaggatgttcggctgctgcaccgccacaatgtgaaggtcgcgtcgcaggttgtgagcaatgcctacctcggctcaggggtggcccccgtgccagagatggtggagcgcggcatggccgtgggcattggaacagatgacgggaattgtaatgactccgtaaacatgatcggagacatgaagtttatggcccatattcaccgcgcggtgcatcgggatgcggacgtgctgaccccagagaagattcttgaaatggcgacgatcgatggggcgcgttcgttgggaatggaccacgagattggttccatcgaaaccggcaagcgcgcggaccttatcctgcttgacctgcgtcaccctcagacgactcctcaccatcatttggcggccacgatcgtgtttcaggcttacggcaatgaggtggacactgtcctgattgacggaaacgttgtgatggagaaccgccgcttgagctttcttccccctgaacgtgagttggcgttccttgaggaagcgcagagccgcgccacagctattttgcagcgggcgaacatggtggctaacccagcttggcgcagcctctag

SEQ ID NO: 95 - DNA, 35 bp, Artificial Sequence
  RF_ptxD_F
ggcgccaccctgcaatatcacgctcgtaaagcgct

SEQ ID NO: 96 - DNA, 34 bp, Artificial Sequence
  RF_ptxD_R
ccgtttgcgtatccagcgctttacgagcgtgata